(19) United States
(12) Patent Application Publication
(10) Pub. No.: US 2010/0160306 A1
(43) Pub. Date: Jun. 24, 2010

(54) Acylated Piperidine Derivatives as Melanocortin-4 Receptor Agonists

(76) Inventors: Raman K. Bakshi, Edison, NJ (US); Qingmei Hong, Scotch Plains, NJ (US); Ravi P. Nargund, East Brunswick, NJ (US)

Correspondence Address: MERCK, P O BOX 2000, RAHWAY, NJ, 07065-0907, US

(21) Appl. No.: 11/992,343

(22) PCT Filed: Sep. 26, 2006
(86) PCT No.: PCT/US06/37243

§ 371 Date: Mar. 20, 2008

Related U.S. Application Data
(60) Provisional application No. 60/722,173, filed on Sep. 30, 2005.

(51) Int. Cl. A61K 31/5386; A61P 3/04; C07D 405/12; A61K 31/453; C07D 401/12; A61K 31/4545; A61K 31/5377; C07D 413/12

(52) U.S. Cl. 514/230.5; 546/207; 514/326; 546/189; 514/316; 514/235.5; 544/130; 544/105

(57) ABSTRACT

Certain novel 4 alkyl substituted N acylated piperidine derivatives are ligands of the human melanocortin receptor(s) and, in particular, are selective ligands of the human melanocortin-4 receptor (MC-4R). They are therefore useful for the treatment, control, or prevention of diseases and disorders responsive to the modulation of MC-4R, such as obesity, diabetes, nicotine addiction, alcoholism, sexual dysfunction, including erectile dysfunction and female sexual dysfunction.

### FIELD OF THE INVENTION

The present invention relates to acylated piperidine derivatives, their synthesis, and their use as melanocortin receptor (MC-R) ligands useful to modulate bodyweight. More particularly, the compounds of the present invention are ligands of the melanocortin-4 receptor (MC-4R) and are thereby useful for the treatment of disorders responsive to the modulation of the melanocortin-4 receptor, such as obesity, diabetes, male sexual dysfunction, female, sexual dysfunction, cachexia, anorexia, wasting, and weight loss.

### BACKGROUND OF THE INVENTION

Obesity is a major health concern in Western societies. It is estimated that about 97 million adults in the United States are overweight or obese. Epidemiological studies have shown that increasing degrees of overweight and obesity are important predictors of decreased life expectancy. Obesity causes or exacerbates many health problems, both independently and in association with other diseases. The medical problems associated with obesity, which can be serious and life-threatening, include hypertension; type 2 diabetes mellitus; elevated plasma insulin concentrations; insulin resistance; dyslipidemias; hyperlipidemia; endometrial, breast, prostate and colon cancer; osteoarthritis; respiratory complications, such as obstructive sleep apnea; cholelithiasis; gallstones; arteriosclerosis; heart disease; abnormal heart rhythms; and heart arrythmias (Kopelman, P. G., Nature 404, 635-643 (2000)). Obesity is further associated with premature death and with a significant increase in mortality and morbidity from stroke, myocardial infarction, congestive heart failure, coronary heart disease, and sudden death.

Pro-opiomelanocortin (POMC) derived peptides are known to affect food intake. Several lines of evidence support the notion that the G-protein coupled receptors (GPCRs) of the melanocortin receptor (MC-R) family, several of which are expressed in the brain, are the targets of POMC derived peptides involved in the control of food intake and metabolism. A specific single MC-R that may be targeted for the control of obesity has not yet been identified, although evidence has been presented that MC-4R signalling is important in mediating feed behavior (S. Q. Giraudo et al., “Feeding effects of hypothalamic injection of melanocortin-4 receptor ligands,” Brain Research, 80: 302-306 (1998)). Evidence for the involvement of MC-R's in obesity includes: i) the agouti (A^{vy}) mouse which ectopically expresses an antagonist of the MC-1R, MC-3R and -4R is obese, indicating that blocking the action of these three MC-R's can lead to hyperphagia and metabolic disorders; ii) MC-4R knockout mice (D. Huszar et al., Cell, 88: 131-141 (1997)) recapitulate the phenotype of the agouti mouse and these mice are obese; iii) the cyclic heptapeptide MT-II (a non-selective MC-1R, -3R, -4R, and -5R agonist) injected intracerebroventricularly (ICV) in rodents, reduces food intake in several animal feeding models (NPY, ob/ob, agouti, fasted) while ICV injected SHU-9119 (MC-3R and 4R antagonist; MC-1R and -5R agonist) reverses this effect and can induce hyperphagia; iv) chronic intraperitoneal treatment of Zucker fatty rats with an α-NDP-MSH derivative (HP228) has been reported to activate MC-1R, -3R, -4R, and -5R and to attenuate food intake and body weight gain over a 12-week period (I. Corcos et al., “HP228 is a potent agonist of melanocortin receptor-4 and significantly attenuates obesity and diabetes in Zucker fatty rats,” Society for Neuroscience Abstracts, 23: 673 (1997)).

Studies have shown that the melanocortin system contributes to the regulation of feeding behavior and bodyweight. Administration of melanocortin antagonists increases food intake and bodyweight, while administration of melanocortin agonists decreases food intake and bodyweight. Support for the role of the MC4R subtype in energy balance is demonstrated by evidence showing that the melanocortin-4 receptor deficiency in humans appears to be the most common monogenetic form of obesity with about 5-6% of obese patients showing this mutation. Furthermore, the severity of the phenotype appears to be greater in individuals that have mutations that result in complete loss of functioning. Based on these findings, the melanocortin system has been targeted for the development of small molecule agonists to treat obesity and small molecule antagonists to treat cachexia.

Weight loss drugs that are currently used in monotherapy for the treatment of obesity have limited efficacy and significant side effects. Studies of the weight loss medications orlistat (Davidson, M. H. et al. (1999) JAMA 281:235-42), dexfenfluramine (Guy Grand, B. et al. (1989) Lancet 2:1142-5), sibutramine (Bray, G. A. et al. (1999) Obes. Res. &:189-98) and phentermine (Douglas, A. et al. (1983) Int. J. Obes. 7:591-5) have demonstrated a limited weight loss of about 5%-10% of body weight for drug compared to placebo. In particular, both sibutramine and orlistat reduce body weight less than 10% over a 6 month or a 1 year period. The side effects of these drugs and anti-obesity agents further limit their use. Dexfenfluramine was withdrawn from the market because of suspected heart valvulopathy; orlistat is limited by gastrointestinal side effects; the use of topiramate is limited by central nervous system effects; and the use of sibutramine is limited by its cardiovascular side effects which have led to reports of deaths and its withdrawal from the market in Italy.

There is a need for a weight loss treatment with enhanced efficacy and fewer undesirable side effects. The instant invention addresses this problem by providing melanocortin receptor (MC-R) agonists, and in particular selective agonists of the melanocortin-4 receptor (MC-4R), useful in the treatment and prevention of obesity and obesity-related disorders, including diabetes.

Melanocortin receptor involvement in male and female sexual dysfunction has also been reported. Approximately 140 million men worldwide suffer from impotency or erectile dysfunction. Current treatment options for erectile dysfunction include phosphodiesterase V inhibitors, such as sildenafil citrate (Viagra®), vardenafil hydrochloride (Levitra®), and tadalafil (Cialis®). Sildenafil is effective in about 70% of patients, however it is contraindicated for patients with unstable heart conditions or cardiovascular disease, in particular patients taking nitrates, such as nitroglycerin, to treat angina. Vardenafil and Tadalafil are also contraindicated for patients taking nitrates and alpha blockers due to the risk of a sudden blood pressure drop resulting in fainting, heart attack or stroke. Other adverse effects associated with the clinical use of these PDE-5 inhibitors include headache, flushing, dyspepsia, dizziness, indigestion, and “abnormal vision, which is characterized by a bluish tinge to vision, but also an increased sensitivity to light or blurred vision. Sildenafil is also being evaluated for the treatment of female sexual dysfunction.

There is a need for a sexual dysfunction treatment with fewer undesirable side effects. The instant invention addresses this problem by providing melanocortin receptor (MC-R) agonists, and in particular selective agonists of the melanocortin-4 receptor (MC-4R), useful in the treatment and prevention of obesity and obesity-related disorders, including diabetes.

Synthetic melanocortin receptor agonists (melanotropic peptides) have been found to initiate erections in men with psychogenic erectile dysfunction. The centrally acting α-melanocyte-stimulating hormone analog, melanotan-II (MT-II), exhibited a 75% response rate when injected intramuscularly or subcutaneously into males with psychogenic erectile dysfunction [See H. Wessells et al., “Synthetic Melanotropic Peptide Initiates Erections in Men With Psychogenic Erectile Dysfunction: Double-Blind, Placebo Controlled Crossover Study,” J. Urol., 160: 389-393 (1998); Fifteenth American Peptide Symposium, Jun. 14-19, 1997 (Nashville Tenn.)]. MT-II (the cyclic heptapeptide Ac-Nle-c[Asp-His-DPhe-Arg-Trp-Lys]-NH₂) is a non-selective MC-1R, -3R, -4R, and -5R agonist (Dory et al., Life Sciences, Vol. 58, 1777-1784, 1996). Adverse reactions observed with MT-II include nausea, flushing, loss of appetite, stretching, and yawning and may be the result of activation of MC-1R, MC-2R, MC-3R, and/or MC-5R. Additionally, MT-II must be administered parenterally, such as by subcutaneous, intravenous, or intramuscular route, since it is not absorbed into the systemic circulation when given by the oral route.

Compositions of melanotropic peptides and methods for the treatment of psychogenic erectile dysfunction are disclosed in U.S. Pat. No. 5,576,290. Methods of stimulating sexual response in females using melanotropic peptides have been disclosed in U.S. Pat. No. 6,051,555. Spiropiperidine, piperidine and piperazine derivatives have been disclosed in WO 99/64002; WO 00/74679; WO 01/70708; WO 01/70337; WO 01/91752; WO 02/059095; WO 02/059107; WO 02/059108; WO 02/059117; WO 02/068387; WO 02/068388; WO 03/007949; WO 03/009847; WO 04/024720; WO 04/089307; WO 04/078717; WO 04/087159; and WO 05/009950 as agonists of the melanocortin receptor(s) and particularly as selective agonists of the MC-4R receptor and thereby useful for the treatment of diseases and disorders, such as obesity, diabetes, and sexual dysfunction, including erectile dysfunction and female sexual dysfunction.

Because of the unresolved deficiencies of the various pharmacological agents discussed above, there is a continuing need in the medical arts for improved methods and compositions to treat individuals suffering from psychogenic and/or organic sexual dysfunction. Such methods should have wider applicability, enhanced convenience and ease of compliance, short onset of action, reasonably long duration of action, and minimal side effects with few contraindications, as compared to agents now available.

It is therefore an object of the present invention to provide acylated piperidine derivatives which are melanocortin receptor agonists and thereby useful to treat obesity, diabetes, male sexual dysfunction, female sexual dysfunction, nicotine addiction and alcoholism.

It is another object of the present invention to provide acylated piperidine derivatives which are selective ligands of the melanocortin-4 (MC-4R) receptor.

It is another object of the present invention to provide pharmaceutical compositions comprising the melanocortin receptor agonists or ligands of the present invention with a pharmaceutically acceptable carrier.

It is another object of the present invention to provide methods for the treatment or prevention of disorders, diseases, or conditions responsive to the modulation of the melanocortin-4 receptor in a subject in need thereof by administering the compounds and pharmaceutical compositions of the present invention.

It is another object of the present invention to provide methods for the treatment or prevention of obesity, diabetes mellitus, male sexual dysfunction, female sexual dysfunction, nicotine addiction and alcoholism by administering the compounds and pharmaceutical compositions of the present invention to a subject in need thereof.

It is another object of the present invention to provide methods for the treatment of erectile dysfunction by administering the compounds and pharmaceutical compositions of the present invention to a subject in need thereof.

These and other objects will become readily apparent from the detailed description that follows.

### SUMMARY OF THE INVENTION

The present invention relates to novel 4-alkyl substituted piperidines of structural formula I:

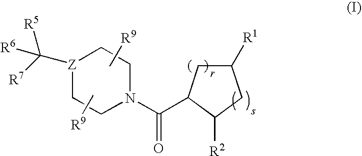

The compounds of structural formula I are effective as melanocortin receptor ligands and are particularly effective as selective ligands of the melanocortin-4 receptor. They are therefore useful for the treatment and/or prevention of disorders responsive to the modulation of the melanocortin-4 receptor, such as obesity, diabetes, obesity-related disorders, nicotine addiction, alcoholism, female sexual dysfunction, and male sexual dysfunction, in particular male erectile dysfunction.

The present invention also relates to pharmaceutical compositions comprising the compounds of the present invention and a pharmaceutically acceptable carrier.

The present invention also relates to methods for the treatment or prevention of disorders, diseases, or conditions responsive to the modulation of the melanocortin-4 receptor in a mammal in need thereof by administering the compounds and pharmaceutical compositions of the present invention.

The present invention further relates to the use of the compounds of the present invention in the preparation of a medicament useful for the treatment or prevention of disorders, diseases, or conditions responsive to the modulation of the melanocortin-4 receptor in a mammal in need thereof by administering the compounds and pharmaceutical compositions of the present invention.

### DETAILED DESCRIPTION OF THE INVENTION

The present invention relates to 4-alkyl substituted N-acylated piperidine derivatives useful as melanocortin receptor modulators, in particular, as selective melanocortin-4 receptor ligands. Compounds of the present invention are described by structural formula I:

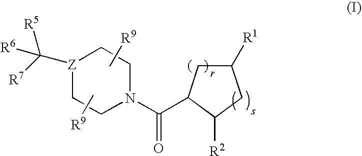

or a pharmaceutically acceptable salt thereof; wherein

### Z is N or CR⁴;

R¹ is selected from the group consisting of:

(1) amidino,

(2) —C₁₋₄alkyliminoyl,

(3) —C₁₋₈ alkyl,

(4) —(CH₂)ₙN(R⁸)₂,

(5) —(CH₂)ₙC₂₋₉heterocycloalkyl,

(6) —(CH₂)ₙC₃₋₈cycloalkyl,

(7) —(CH₂)ₙphenyl,

(8) —(CH₂)ₙnaphthyl,

(9) —(CH₂)ₙheteroaryl,

(10) —(CH₂)ₙC(O)C₁₋₈ alkyl,

(11) —(CH₂)ₙC(O)C₃₋₈cycloalkyl,

(12) —(CH₂)ₙC(O)C₂₋₉heterocycloalkyl,

(13) —(CH₂)ₙC(O)phenyl,

(14) —(CH₂)ₙC(O)naphthyl,

(15) —(CH₂)ₙC(O)heteroaryl,

(16) —(CH₂)ₙCO₂H,

(17) —(CH₂)ₙCO₂C₁₋₈ alkyl,

(18) —(CH₂)ₙCO₂C₃₋₈cycloalkyl,

(19) —(CH₂)ₙCO₂C₂₋₉heterocycloalkyl,

(20) —(CH₂)ₙCO₂-phenyl,

(21) —(CH₂)ₙCO₂naphthyl,

(22) —(CH₂)ₙCO₂heteroaryl,

wherein phenyl, naphthyl, and heteroaryl are unsubstituted or substituted with one to three substituents independently selected from R³, and alkyl, cycloalkyl, heterocycloalkyl and (CH₂)ₙ are unsubstituted or substituted with one to three substituents independently selected from R³ and oxo;
R² is selected from the group consisting of:

(1) phenyl,

(2) naphthyl, and

(3) heteroaryl,

wherein phenyl, naphthyl, and heteroaryl are unsubstituted or substituted with one to three substituents independently selected from R¹⁰;
each R³ is independently selected from the group consisting of:

(1) —C₁₋₈ alkyl,

(2) —(CH₂)ₙ-phenyl,

(3) —(CH₂)ₙ-heteroaryl,

(4) —(CH₂)ₙC₂₋₉heterocycloalkyl,

(5) —(CH₂)ₙC₃₋₇ cycloalkyl,

(6) halogen,

(7) —OR⁸,

(8) —(CH₂)ₙC≡N,

(9) —(CH₂)ₙN(R⁸)₂,

(10) —(CH₂)ₙC(O)N(R⁸)₂,

(11) —(CH₂)ₙC(O)NR⁸N(R⁸)₂,

(12) —(CH₂)ₙC(O)NR⁸NR⁸C(O)R⁸, and

(13) —(CH₂)ₙCF₃,

wherein phenyl and heteroaryl are unsubstituted or substituted with one to three substituents independently selected from halogen, hydroxy, C₁₋₄alkyl, trifluoromethyl, and C₁₋₄alkoxy, and wherein any alkyl, cycloalkyl, heterocycloalkyl, and methylene (CH₂) carbon atom in R³ is unsubstituted or substituted with one to two substituents independently selected from halogen, hydroxy, oxo, C₁₋₄ alkyl, trifluoromethyl, and C₁₋₄ alkoxy, or two R³ substituents on the same carbon atom are taken together with the carbon atom to form a cyclopropyl group;
R⁴ is selected from the group consisting of:

(1) hydrogen,

(2) —C₁₋₆ alkyl, and

(3) —OC₁₋₆ alkyl;

R⁵ is selected from the group consisting of:

(1) —CF₃,

(2) —C₁₋₆ alkyl,

(3) —C₂₋₈ alkenyl,

(4) —C₂₋₈ alkynyl,

(5) —OC₁₋₈ alkyl,

(6) —(CH₂)ₙC₃₋₈cycloalkyl,

(7) —(CH₂)ₙC₂₋₉heterocycloalkyl,

(8) —(CH₂)ₙ-phenyl,

(9) —(CH₂)ₙ-naphthyl, and

(10) —(CH₂)ₙheteroaryl,

wherein phenyl, naphthyl, and heteroaryl are unsubstituted or substituted with one to three substituents independently selected from R³, and alkyl, alkenyl, alkynyl, cycloalkyl and heterocycloalkyl are unsubstituted or substituted with one to three substituents independently selected from R³ and oxo, and wherein any methylene (CH₂)ₙ in R⁵ is unsubstituted or substituted with one to two substituents independently selected from halogen, hydroxy, oxo, and C_{1-—}4 alkyl;
R⁶ is selected from the group consisting of:

(1) hydrogen,

(2) —C₁₋₆ alkyl, and

(3) —OC₁₋₆ alkyl;

R⁷ is selected from the group consisting of:

(1) —(CH₂)ₙN(R⁸)₂,

(2) —(CH₂)ₙNR⁸C(O)R⁸,

(3) —(CH₂)ₙOR⁸,

(4) —(CH₂)ₙC≡N,

(5) —(CH₂)ₙC(O)OR⁸,

(6) —(CH₂)ₙC(O)N(R⁸)₂,

(7) —(CH₂)ₙNR⁸C(O)N(R⁸)₂,

(8) —(CH₂)ₙNR⁸C(O)heteroaryl,

(9) —(CH₂)ₙheteroaryl,

(10) —(CH₂)ₙNR⁸S(O)ₚR⁸,

(11) —(CH₂)ₙSR⁸, and

(12) —(CH₂)ₙS(O)ₚR⁸,

wherein heteroaryl is unsubstituted or substituted with one to three substituents selected from C₁₋₄alkyl, and any methylene (CH₂) in R⁷ is unsubstituted or substituted with one to two substituents independently selected from halogen, hydroxy, oxo, and C₁₋₄alkyl, or two C₁₋₄alkyl substituents on any methylene (CH₂) in R⁷ together with the atom to which they are attached form a 3, 4, 5, or 6-membered ring optionally containing an additional heteroatom selected from O, S, —NH, and —NC₁₋₄ alkyl;
each R⁸ is independently selected from the group consisting of:

(1) hydrogen,

(2) —C₁₋₈ alkyl,

(3) —C₂₋₈ alkenyl,

(4) —C₂₋₈ alkynyl,

(5) —C₁₋₈ alkyl,

(6) —(CH₂)ₙC₃₋₈cycloalkyl,

(7) —(CH₂)ₙC₂₋₉heterocycloalkyl,

(8) —(CH₂)ₙ-phenyl,

(9) —(CH₂)ₙ-naphthyl, and

(10) —(CH₂)ₙheteroaryl,

wherein phenyl, naphthyl, and heteroaryl, alkyl, alkenyl, alkynyl, cycloalkyl and heterocycloalkyl are unsubstituted or substituted with one to three substituents independently selected from —N(C₁₋₆alkyl)₂, —NH₂, NH(C₁₋₆ alkyl), halogen, C₁₋₆alkyl, C₁₋₆alkoxy, hydroxy, and oxo, and wherein any methylene (CH₂) in R⁸ is unsubstituted or substituted with one to two substituents independently selected from halogen, hydroxy, oxo, and C₁₋₄ alkyl;
each R⁹ is independently selected from the group consisting of:

(1) hydrogen,

(2) —OH,

(3) C₁₋₈alkyl,

(4) —OC₁₋₈alkyl,

(5) halogen;

(6) —NR₅,

(7) —SR⁵, and

(8) —CF₃,

wherein two C₁₋₈alkyl substituents along with the atoms to which they are attached can form a 4- to 8-membered ring;
each R¹⁰ is independently selected from the group consisting of:

(1) —C₁₋₈ alkyl,

(2) —C₂₋₈ alkenyl,

(3) —(CH₂)ₙ-phenyl,

(4) —(C₁₋₁₂)ₙ-naphthyl,

(5) —(CH₂)ₙ-heteroaryl,

(6) —(CH₂)ₙC₂₋₉heterocycloalkyl,

(7) —(CH₂)ₙC₃₋₇ cycloalkyl,

(8) halogen, and

(9) —OR⁸,

wherein alkenyl, phenyl, naphthyl, and heteroaryl are unsubstituted or substituted with one to three substituents independently selected from halogen, hydroxy, C₁₋₄ alkyl, trifluoromethyl, and C₁₋₄alkoxy, and wherein alkyl, cycloalkyl, heterocycloalkyl, and any methylene (CH₂) carbon atom in R¹⁰ are unsubstituted or substituted with one to two substituents independently selected from halogen, hydroxy, oxo, C₁₋₄alkyl, trifluoromethyl, and C₁₋₄alkoxy, or two R¹⁰ substituents on the same carbon atom are taken together with the carbon atom to form a cyclopropyl group;
r is 1 or 2;
s is 0, 1 or 2;
n is 0, 1, 2, 3, or 4; and
p is 0, 1, or 2.

In another embodiment of the compounds of the present invention, there are provided compounds of structural formula IIa or IIb of the indicated relative stereochemical configurations having the trans orientation of the phenyl and piperazinecarbonyl substituents:

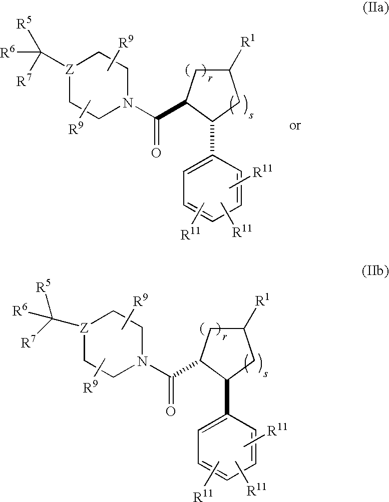

or a pharmaceutically acceptable salt thereof, wherein

### Z is N or CR⁴;

R¹ is selected from the group consisting of:

(1) amidino,

(2) —C₁₋₄alkyliminoyl,

(3) —C_{1-—}8 alkyl,

(4) —(CH₂)ₙN(R⁸)₂,

(5) —(CH₂)ₙC₂₋₉heterocycloalkyl,

(6) —(CH₂)ₙC₃₋₈cycloalkyl,

(7) —(CH₂)ₙphenyl,

(8) —(CH₂)ₙnaphthyl,

(9) —(CH₂)ₙheteroaryl,

(10) —(CH₂)ₙC(O)C₁₋₈ alkyl,

(11) —(C₁₋₁₂)ₙC(O)C₃₋₈cycloalkyl,

(12) —(CH₂)ₙC(O)C₂₋₉heterocycloalkyl,

(13) —(CH₂)ₙC(O)phenyl,

(14) —(CH₂)ₙC(O)naphthyl,

(15) —(CH₂)ₙC(O)heteroaryl,

(16) —(CH₂)ₙCO₂H,

(17) —(CH₂)ₙCO₂C₁₋₈ alkyl,

(18) —(CH₂)ₙCO₂C₃₋₈cycloalkyl,

(19) —(CH₂)ₙCO₂C₂₋₉heterocycloalkyl,

(20) —(CH₂)ₙCO₂-phenyl,

(21) —(CH₂)ₙCO₂naphthyl,

(22) —(CH₂)ₙCO₂heteroaryl,

wherein phenyl, naphthyl, and heteroaryl are unsubstituted or substituted with one to three substituents independently selected from R³, and alkyl, cycloalkyl, heterocycloalkyl and (CH₂)ₙ are unsubstituted or substituted with one to three substituents independently selected from R³ and oxo;
R² is selected from the group consisting of:

(1) phenyl,

(2) naphthyl, and

(3) heteroaryl,

wherein phenyl, naphthyl, and heteroaryl are unsubstituted or substituted with one to three substituents independently selected from R¹⁰;
each R³ is independently selected from the group consisting of:

(1) —C₁₋₈ alkyl,

(2) —(CH₂)ₙ-phenyl,

(3) —(CH₂)ₙ-heteroaryl,

(4) —(CH₂)ₙC₂₋₉heterocycloalkyl,

(5) —(CH₂)ₙC₃₋₇ cycloalkyl,

(6) halogen,

(7) —OR⁸,

(8) —(CH₂)ₙC≡N,

(9) —(CH₂)ₙN(R⁸)₂,

(10) —(CH₂)ₙC(O)N(R⁸)₂,

(11) —(CH₂)ₙC(O)NR⁸N(R⁸)₂,

(12) —(CH₂)ₙC(O)NR⁸NR⁸C(O)R⁸, and

(13) —(CH₂)ₙCF₃,

wherein phenyl and heteroaryl are unsubstituted or substituted with one to three substituents independently selected from halogen, hydroxy, C_{1-—}4 alkyl, trifluoromethyl, and C₁₋₄alkoxy, and wherein any alkyl, cycloalkyl, heterocycloalkyl, and methylene (CH₂) carbon atom in R³ is unsubstituted or substituted with one to two substituents independently selected from halogen, hydroxy, oxo, C₁₋₄ alkyl, trifluoromethyl, and C₁₋₄ alkoxy, or two R³ substituents on the same carbon atom are taken together with the carbon atom to form a cyclopropyl group;
R⁴ is selected from the group consisting of:

(1) hydrogen,

(2) —C₁₋₆ alkyl, and

(3) —OC₁₋₆ alkyl;

R⁵ is selected from the group consisting of:

(1) —CF₃,

(2) —C₁₋₆ alkyl,

(3) —C₂₋₈ alkenyl,

(4) —C₂₋₈ alkynyl,

(5) —OC₁₋₈ alkyl,

(6) —(CH₂)ₙC₃₋₈cycloalkyl,

(7) —(CH₂)ₙC₂₋₉heterocycloalkyl,

(8) —(CH₂)ₙ-phenyl,

(9) —(CH₂)ₙ-naphthyl, and

(10) —(CH₂)ₙheteroaryl,

wherein phenyl, naphthyl, and heteroaryl are unsubstituted or substituted with one to three substituents independently selected from R³, and alkyl, alkenyl, alkynyl, cycloalkyl and heterocycloalkyl are unsubstituted or substituted with one to three substituents independently selected from R³ and oxo, and wherein any methylene (CH₂) in R⁵ is unsubstituted or substituted with one to two substituents independently selected from halogen, hydroxy, oxo, and C₁₋₄ alkyl;
R⁶ is selected from the group consisting of:

(1) hydrogen,

(2) —C₁₋₆ alkyl, and

(3) —OC₁₋₆ alkyl;

R⁷ is selected from the group consisting of:

(1) —(CH₂)ₙN(R⁸)₂,

(2) —(CH₂)ₙNR⁸C(O)R⁸,

(3) —(CH₂)ₙOR⁸,

(4) —(CH₂)ₙC≡N,

(5) —(CH₂)ₙC(O)OR⁸,

(6) —(CH₂)ₙC(O)N(R⁸)₂,

(7) —(CH₂)ₙNR⁸C(O)N(R⁸)₂,

(8) —(CH₂)ₙNR⁸C(O)heteroaryl,

(9) —(CH₂)ₙheteroaryl,

(10) —(CH₂)ₙNR⁸S(O)ₚR⁸,

(11) —(CH₂)ₙSR⁸, and

(12) —(CH₂)ₙS(O)ₚR⁸,

wherein heteroaryl is unsubstituted or substituted with one to three substituents selected from C₁₋₄ alkyl, and any methylene (CH₂) in R⁷ is unsubstituted or substituted with one to two substituents independently selected from halogen, hydroxy, oxo, and C₁₋₄alkyl, or two C₁₋₄alkyl substituents on any methylene (CH₂) in R⁷ together with the atom to which they are attached form a 3, 4, 5, or 6-membered ring optionally containing an additional heteroatom selected from O, S, —NH, and —NC₁₋₄ alkyl;
each R⁸ is independently selected from the group consisting of:

(1) hydrogen,

(2) —C₁₋₈ alkyl,

(3) —C₂₋₈ alkenyl,

(4) —C₂₋₈ alkynyl,

(5) —OC₁₋₈ alkyl,

(6) —(CH₂)ₙC₃₋₈cycloalkyl,

(7) —(C112)ₙC₂₋₉heterocycloalkyl,

(8) —(CH₂)ₙ-phenyl,

(9) —(CH₂)ₙ-naphthyl, and

(10) —(CH₂)ₙheteroaryl,

wherein phenyl, naphthyl, and heteroaryl, alkyl, alkenyl, alkynyl, cycloalkyl and heterocycloalkyl are unsubstituted or substituted with one to three substituents independently selected from N(C₁₋₆alkyl)₂, —NH₂, NH(C₁₋₆ alkyl), halogen, C₁₋₆alkyl, C₁₋₆alkoxy, hydroxy and oxo, and wherein any methylene (CH₂) in R⁸ is unsubstituted or substituted with one to two substituents independently selected from halogen, hydroxy, oxo, and C₁₋₄alkyl;
each R⁹ is independently selected from the group consisting of:

(1) hydrogen,

(2) —OH,

(3) C₁₋₈alkyl,

(4) —OC₁₋₈alkyl,

(5) halogen;

(6) —NR⁵,

(7) —SR⁵, and

(8) —CF₃,

wherein two C₁₋₈alkyl substituents along with the atoms to which they are attached can form a 4- to 8-membered ring;
each R¹¹ is independently selected from the group consisting of:

(1) hydrogen,

(2) —C₁₋₈ alkyl,

(3) —C₂₋₈ alkenyl,

(4) —(CH₂)ₙ-phenyl,

(5) —(CH₂)ₙ-naphthyl,

(6) —(CH₂)ₙ-heteroaryl,

(7) —(CH₂)ₙC₂₋₉heterocycloalkyl,

(8) —(CH₂)ₙC₃₋₇ cycloalkyl,

(9) halogen,

(10) —OR⁸,

wherein alkenyl, phenyl, naphthyl, and heteroaryl are unsubstituted or substituted with one to three substituents independently selected from halogen, hydroxy, C₁₋₄alkyl, trifluoromethyl, and C₁₋₄ alkoxy, and wherein alkyl, cycloalkyl, heterocycloalkyl, and any methylene (CH₂) carbon atom in R¹¹ are unsubstituted or substituted with one to two substituents independently selected from halogen, hydroxy, oxo, C₁₋₄ alkyl, trifluoromethyl, and C₁₋₄ alkoxy, or two R¹¹ substituents on the same carbon atom are taken together with the carbon atom to form a cyclopropyl group;
r is 1 or 2;
s is 0, 1 or 2;
n is 0, 1, 2, 3, or 4; and
p is 0, 1, or 2.

In a class of the embodiments of the present invention, Z is N.

In another class of the embodiments of the present invention Z is CR⁴.

In another class of the embodiments of the present invention, R¹ is selected from the group consisting of: amidino, —C₁₋₄alkyliminoyl, —C₁₋₈ alkyl, —(CH₂)ₙN(R⁸)₂, —(CH₂)ₙC₂₋₉heterocycloalkyl, —(CH₂)ₙC₃₋₈cycloalkyl, —(CH₂)ₙphenyl, —(CH₂)ₙnaphthyl, and —(CH₂)ₙheteroaryl. In a subclass of this class, R¹ is —(CH₂)ₙN(R⁸)₂. In another subclass of this class, R¹ is —(CH₂)ₙC₂₋₉heterocycloalkyl. In another subclass of this class, R¹ is —(CH₂)ₙheteroaryl.

In another class of the embodiments of the present invention, R² is phenyl unsubstituted or substituted with one to three substituents independently selected from R¹⁰. In a subclass of this class, R² is phenyl substituted with one to three substituents independently selected from R¹⁰. In another subclass of this class, R² is phenyl substituted with two substituents independently selected from R¹⁰. In another subclass of this class, R² is 2,6-difluorophenyl.

In another class of the embodiments of the present invention, each R³ is independently selected from the group consisting of: —C₁₋₈ alkyl, halogen, and —(CH₂)ₙN(R⁸)₂, wherein alkyl, and methylene (CH₂) carbon atom in R³ is unsubstituted or substituted with one to two substituents independently selected from halogen, hydroxy, oxo, C₁₋₄alkyl, trifluoromethyl, and C₁₋₄ alkoxy, or two R³ substituents on the same carbon atom are taken together with the carbon atom to form a cyclopropyl group. In a subclass of this class, R³ is —C₁₋₈ alkyl. In another subclass of this class, R³ is halogen. In another subclass of this class, R³ is —(CH₂)ₙN(R⁸)₂.

In another class of the embodiments of the present invention, R⁴ is hydrogen. In another class of this embodiment, R⁴ is —C₁₋₆alkyl.

In another class of the embodiments of the present invention, R⁵ is selected from the group consisting of: —C₁₋₆ alkyl, and —(CH₂)₀₋₁C₃₋₈cycloalkyl, wherein alkyl, and cycloalkyl are unsubstituted or substituted with one to three substituents independently selected from R³ and oxo, and wherein any methylene (CH₂) in R⁵ is unsubstituted or substituted with one to two substituents independently selected from halogen, hydroxy, oxo, and C_{1 —}4 alkyl. In a subclass of this class, R⁵ is —C₁₋₆ alkyl. In another subclass of this class, R⁵ is —(CH₂)₀₋₁C₃₋₈cycloalkyl. In another subclass of this class, R⁵ is selected from the group consisting of: —CF₃, —(CH₂)C(CH₃)₃; —(CH₂)₀₋₁CH(CH₃)₂, —CH(CH₂CH₃)₂, cyclobutyl, -cyclopentyl, -cyclohexyl, and -phenyl, wherein phenyl is unsubstituted or substituted with one to three substituents independently selected from R³, wherein the alkyl and cycloalkyl groups are unsubstituted or substituted with one to three substituents independently selected from R³ and oxo, and wherein any methylene (CH₂) in R⁵ is unsubstituted or substituted with one to two substituents independently selected from halogen, hydroxy, oxo, and C₁₋₄ alkyl. In yet another subclass of this class, R⁵ is selected from the group consisting of: —(CH₂)C(CH₃)₃ and -cyclohexyl, wherein cyclohexyl is unsubstituted or substituted with one to three substituents independently selected from R³ and oxo, and wherein any methylene (CH₂) in R⁵ is unsubstituted or substituted with one to two substituents independently selected from halogen, hydroxy, oxo, and C₁₋₄alkyl.

In another class of the embodiments of the present invention, R⁶ is hydrogen. In yet another class of this embodiment, R⁶ is —C₁₋₆alkyl.

In another class of the embodiments of the present invention, R⁷ is selected from the group consisting of: —(CH₂)₀₋₂NR⁸C(O)R⁸, —(CH₂)₀₋₂OR⁸, —(CH₂)₀₋₂C≡N, —(CH₂)₀₋₂C(O)OR⁸, —(CH₂)ₙC(O)N(R⁸)₂, —(CH₂)₀₋₂NR⁸C(O)N(R⁸)₂, —(CH₂)₀₋₂NR⁸C(O)heteroaryl, —(CH₂)₀₋₂heteroaryl, —(CH₂)ₙNR⁸S(O)₂R⁸, wherein heteroaryl is unsubstituted or substituted with one to three substituents selected from C₁₋₄ alkyl; and any methylene (CH₂) in R⁷ is unsubstituted or substituted with one to two substituents independently selected from halogen, hydroxy, oxo, and C₁₋₄ alkyl, or two C₁₋₄ alkyl substituents on any methylene (CH₂) in R⁷ together with the atom to which they are attached form a 3, 4, 5, or 6-membered ring optionally containing an additional heteroatom selected from O, S, —NH, and —NC₁₋₄alkyl. In a subclass of this class, R⁷ is selected from the group consisting of: —(CH₂)₀₋₂NR⁸C(O)R⁸, and —(CH₂)₀₋₂NR⁸S(O)₂R⁸, wherein any methylene (CH₂) in R⁷ is unsubstituted or substituted with one to two substituents independently selected from halogen, hydroxy, oxo, and C₁₋₄ alkyl, or two C₁₋₄ alkyl substituents on any methylene (CH₂) in R⁷ together with the atom to which they are attached form a 3, 4, 5, or 6-membered ring optionally containing an additional heteroatom selected from O, S, —NH, and —NC₁₋₄alkyl. In a subclass of this subclass, R⁷ is —(CH₂)₂NR⁸C(O)R⁸, wherein any methylene (CH₂) in R⁷ is unsubstituted or substituted with one to two substituents independently selected from halogen, hydroxy, oxo, and C₁₋₄ alkyl. In another subclass of this subclass, R⁷ is —(CH₂)NR⁸S(O)₂R⁸, wherein the methylene (CH₂) in R⁷ is unsubstituted or substituted with one to two substituents independently selected from halogen, hydroxy, oxo, and C₁₋₄ alkyl.

In another class of the embodiments of the present invention, R⁸ is hydrogen or —C₁₋₈ alkyl wherein alkyl is unsubstituted or substituted with one to three substituents independently selected from N(C₁₋₆alkyl)₂, —NH₂, NH(C₁₋₆ alkyl), halogen, C₁₋₆alkyl, C₁₋₆alkoxy, hydroxy, and oxo, and wherein any methylene (CH₂) in R⁸ is unsubstituted or substituted with one to two substituents independently selected from halogen, hydroxy, oxo, and C₁₋₄ alkyl.

In another class of the embodiments of the present invention, R⁹ is independently selected from the group consisting of: C₁₋₆ alkyl, and hydrogen, wherein two C₁₋₆alkyl substituents along with the atoms to which they are attached can form a 4- to 8-membered ring. In a subclass of this class, R⁹ methyl. In another subclass of this class, R⁹ is hydrogen.

In another class of the embodiments of the present invention, R¹⁰ is selected from the group consisting of: —C₁₋₈ alkyl, halogen, —OR⁸, —(CH₂)ₙC≡N, —(CH₂)ₙS(O)ₚR⁸, and —CF₃, wherein any alkyl and methylene (CH₂) carbon atom in R¹⁰ is unsubstituted or substituted with one to two substituents independently selected from halogen, hydroxy, oxo, C₁₋₄ alkyl, trifluoromethyl, and C₁₋₄ alkoxy. In a subclass of this class, R¹⁰ is selected from the group consisting of: —C₁₋₈ alkyl, fluoro, chloro, —OCH₃, —NO₂, —C≡N, —S(O)₀₋₁R⁸, and —CF₃, wherein any alkyl is unsubstituted or substituted with one to two substituents independently selected from halogen, hydroxy, oxo, C₁₋₄ alkyl, trifluoromethyl, and C₁₋₄ alkoxy. In another subclass of this class, R¹⁰ is chloro, bromo, fluoro, —CF₃, —SC₁₋₆alkyl, —OC₁₋₆alkyl, —C₁₋₆alkyl, —NO₂, aryl, and heteroaryl. In a subclass of this subclass, R¹⁰ is fluoro.

In another class of the embodiments of the present invention, R¹¹ is selected from the group consisting of: hydrogen, —C₁₋₈ alkyl, halogen, —OR⁸, —(CH₂)ₙC≡N, —(CH₂)ₙS(O)ₚR⁸, and —CF₃, wherein any alkyl and methylene (CH₂) carbon atom in R¹¹ is unsubstituted or substituted with one to two substituents independently selected from halogen, hydroxy, oxo, C₁₋₄ alkyl, trifluoromethyl, and C₁₋₄ alkoxy, or two R¹¹ substituents on the same carbon atom are taken together with the carbon atom to form a cyclopropyl group. In a subclass of this class, R¹¹ is selected from the group consisting of: hydrogen, —C₁₋₈ alkyl, fluoro, chloro, —OCH₃, —NO₂, —C≡N, —S(O)₀₋₁R⁸, and —CF₃, wherein any alkyl is unsubstituted or substituted with one to two substituents independently selected from halogen, hydroxy, oxo, C₁₋₄alkyl, trifluoromethyl, and C₁₋₄ alkoxy, or two R¹¹ substituents on the same carbon atom are taken together with the carbon atom to form a cyclopropyl group. In another subclass of this class, R¹¹ is chloro, bromo, fluoro, —CF₃, —SC₁₋₆alkyl, —OC₁₋₆alkyl, —C₁₋₆alkyl, —NO₂, aryl, and heteroaryl. In a subclass of this subclass, R¹¹ is fluoro.

In another class of the embodiments of the present invention, r is 1 and s is 1. In another class of the embodiments of the present invention, r is 2 and s is 1.

In another class of the embodiments of the present invention, n is 0, 1, and 2. In another class of the embodiments of the present invention, p is 2.

Illustrative, but nonlimiting, examples of compounds of the present invention that are useful as melanocortin-4 receptor agonists are the following:

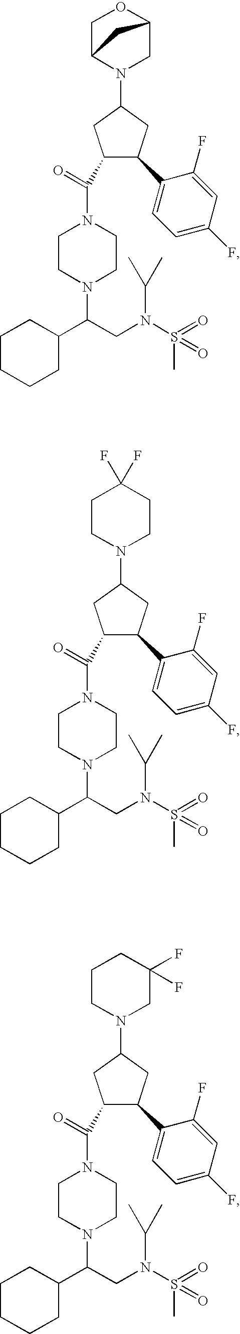

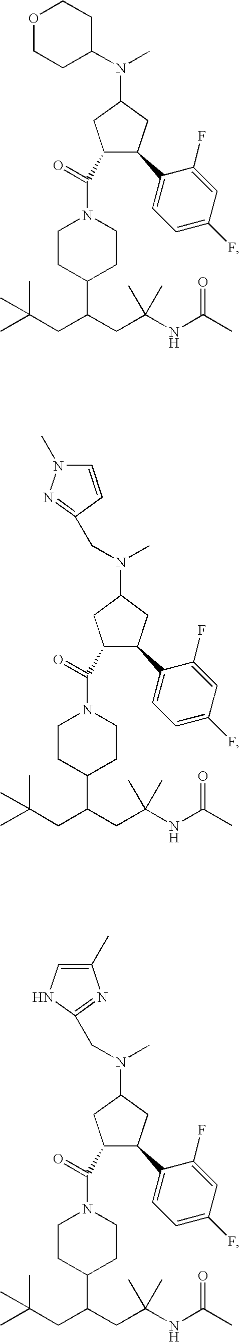

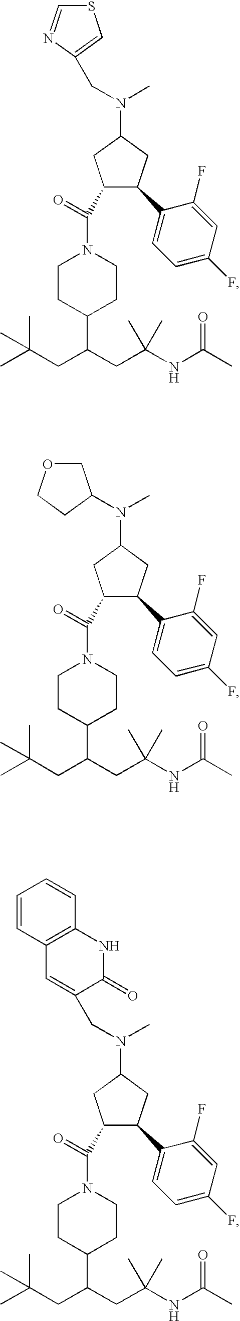

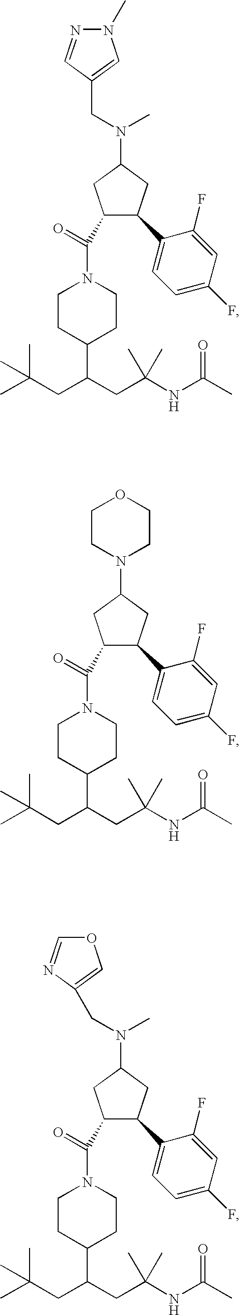

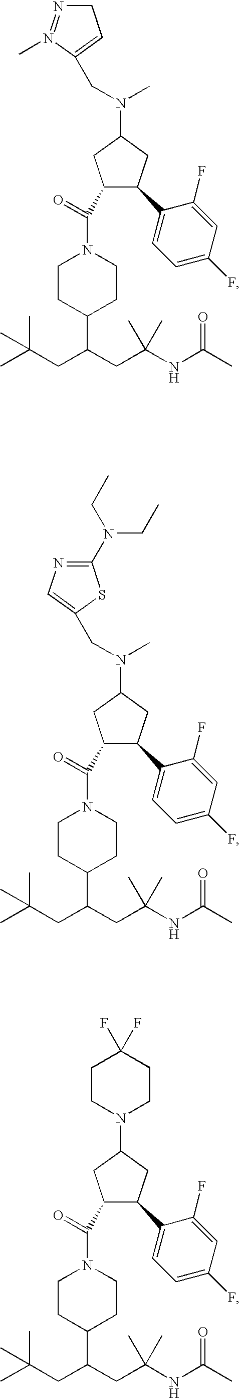

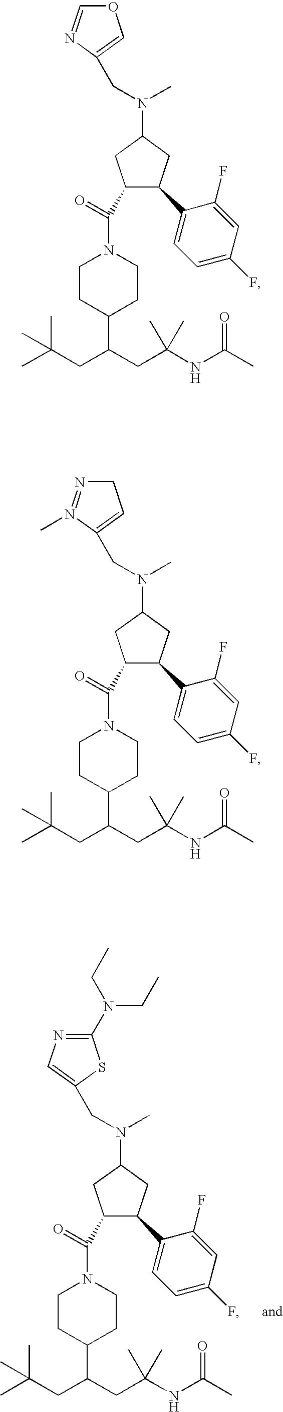

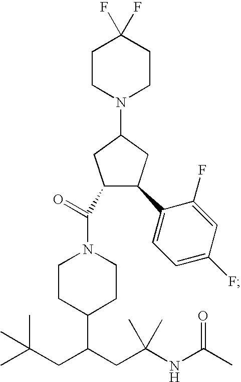

and pharmaceutically acceptable salts thereof.

The compounds of structural formula I are effective as melanocortin receptor ligands and are particularly effective as selective ligands of the melanocortin-4 receptor. They are therefore useful for the treatment and/or prevention of disorders responsive to the modulation of the melanocortin-4 receptor, such as obesity, diabetes, obesity-related disorders, nicotine addiction, alcoholism, as well as male and female sexual dysfunction, and in particular male erectile dysfunction, cachexia, wasting, anorexia and weight loss.

More particularly, the selective melanocortin-4 receptor (MC-4R) agonists of formula I are useful for the treatment of disorders responsive to the activation of the melancortin-4 receptor, such as obesity, diabetes, nicotine addiction, alcoholism, male sexual dysfunction, and female sexual dysfunction. Furthermore, the selective melanocortin-4 receptor (MC-4R) antagonists of formula I are useful for the treatment of disorders responsive to the deactivation of the melanocortin-4 receptor, such as cachexia, wasting, anorexia, frailty, sarcopenia and weight loss.

Another aspect of the present invention provides a method for the treatment or prevention of obesity, diabetes, or an obesity related disorder in a subject in need thereof which comprises administering to said subject a therapeutically or prophylactically effective amount of a melanocortin-4 receptor agonist of the present invention. Another aspect of the present invention provides a method for the treatment or prevention of obesity in a subject in need thereof which comprises administering to the subject a therapeutically or prophylactically effective amount of a compound according to claim 1, or a pharmaceutically acceptable salt thereof. Another aspect of the present invention provides a method for the treatment or prevention of diabetes mellitus in a subject in need thereof comprising administering to the subject a therapeutically or prophylactically effective amount of a compound according to claim 1, or a pharmaceutically acceptable salt thereof. Another aspect of the present invention provides a method for the treatment or prevention of an obesity-related disorder selected from the group consisting of overeating, binge eating, and bulimia, hypertension, elevated plasma insulin concentrations, insulin resistance, dyslipidemias, hyperlipidemia, endometrial, breast, prostate and colon cancer, osteoarthritis, obstructive sleep apnea, cholelithiasis, gallstones, heart disease, abnormal heart rhythms and arrythmias, myocardial infarction, congestive heart failure, coronary heart disease, sudden death, stroke, polycystic ovary disease, craniopharyngioma, the Prader-Willi Syndrome, Frohlich's syndrome, GH-deficient subjects, normal variant short stature, Turner's syndrome, metabolic syndrome, insulin resistance syndrome, sexual and reproductive dysfunction, infertility, hypogonadism, hirsutism, obesity-related gastro-esophageal reflux, Pickwickian syndrome, cardiovascular disorders, inflammation, systemic inflammation of the vasculature, arteriosclerosis, hypercholesterolemia, hyperuricaemia, lower back pain, gallbladder disease, gout, and kidney cancer, cardiac hypertrophy, left ventricular hypertrophy, nicotine addiction and alcoholism, in a subject in need thereof which comprises administering to the subject a therapeutically or prophylactically effective amount of a compound according to claim 1, or a pharmaceutically acceptable salt thereof.

The present invention also relates to methods for treating or preventing obesity by administering the melanocortin-4 receptor agonist of the present invention in combination with a therapeutically or prophylactically effective amount of another agent known to be useful to treat or prevent the condition. The present invention also relates to methods for treating or preventing diabetes by administering the melanocortin-4 receptor agonist of the present invention in combination with a therapeutically or prophylactically effective amount of another agent known to be useful to treat or prevent the condition.

Another aspect of the present invention provides a method for the treatment or prevention of female or male sexual dysfunction, including male erectile dysfunction, which comprises administering to a subject in need of such treatment or prevention a therapeutically or prophylactically effective amount of a melanocortin-4 receptor agonist of the present invention. Another aspect of the present invention provides a method for the treatment or prevention of erectile dysfunction in a subject in need thereof comprising administering to the subject a therapeutically or prophylactically effective amount of a compound according to claim 1, or a pharmaceutically acceptable salt thereof. The present invention also relates to methods for treating or preventing erectile dysfunction by administering the melanocortin-4 receptor agonist of the present invention in combination with a therapeutically or prophylactically effective amount of another agent known to be useful to treat the condition.

Another aspect of the present invention provides a method for the treatment or prevention of alcoholism which comprises administering to a subject in need of such treatment or prevention a therapeutically or prophylactically effective amount of a melanocortin 4 receptor agonist of the present invention. The present invention also provides a method for reducing alcohol consumption which comprises administering to a subject in need of such treatment or prevention a therapeutically or prophylactically effective amount of a melanocortin 4 receptor agonist of the present invention.

Another aspect of the present invention provides a method for the treatment or prevention of nicotine addiction which comprises administering to a subject in need of such treatment or prevention a therapeutically or prophylactically effective amount of a melanocortin 4 receptor agonist of the present invention. The present invention also provides a method for reducing nicotine consumption which comprises administering to a subject in need of such treatment a therapeutically effective amount of a melanocortin 4 receptor agonist of the present invention. Yet another aspect of the present invention provides a method for the treatment or prevention of substance addiction which comprises administering to a subject in need of such treatment or prevention a therapeutically or prophylactically effective amount of a melanocortin 4 receptor agonist of the present invention.

Yet another aspect of the present invention provides a method for the treatment or prevention of cachexia which comprises administering to a subject in need of such treatment or prevention a therapeutically or prophylactically effective amount of a melanocortin 4 receptor antagonist of the present invention. The present invention also provides a method for the treatment or prevention of anorexia, wasting or weight loss which comprises administering to a subject in need of such treatment or prevention a therapeutically or prophylactically effective amount of a melanocortin 4 receptor antagonist of the present invention.

Another aspect of the present invention provides a pharmaceutical composition comprising a compound of structural formula I and a pharmaceutically acceptable carrier.

Yet another aspect of the present invention relates to the use of a compound of structural formula I for the manufacture of a medicament useful for the treatment or prevention, or suppression of a disease mediated by the melanocortin-4 receptor in a subject in need thereof.

Yet another aspect of the present invention relates to the use of a melanocortin-4 agonist of the present invention for the manufacture of a medicament useful for the treatment or prevention, or suppression of a disease mediated by the melanocortin-4 receptor, wherein the disease is selected from the group consisting of obesity, diabetes and an obesity-related disorder in a subject in need thereof.

Yet another aspect of the present invention relates to the use of a melanocortin-4 agonist of the present invention for the manufacture of a medicament useful for the treatment or prevention, or suppression of male and female sexual dysfunction, and male erectile dysfunction in a subject in need thereof.

Yet another aspect of the present invention relates to the use of a selective melanocortin-4 agonist of the present invention in the preparation of a medicament useful for treating or preventing alcoholism in a subject in need thereof. The present invention also relates to the use of a selective melanocortin-4 agonist of the present invention in the preparation of a medicament useful for reducing alcohol consumption in a subject in need thereof.

Yet another aspect of the present invention relates to the use of a selective melanocortin 4 receptor agonist of the present invention in the preparation of a medicament useful to treat or prevent nicotine addiction in a subject in need thereof. The present invention also relates to the use of a selective melanocortin 4 receptor agonist of the present invention in the preparation of a medicament useful to reduce nicotine consumption in a subject in need thereof.

Yet another aspect of the present invention relates to the use of a selective melanocortin 4 receptor agonist of the present invention in the preparation of a medicament useful to treat substance addiction in a subject in need thereof.

Yet another aspect of the present invention relates to the use of a selective melanocortin 4 receptor antagonist of the present invention in the preparation of a medicament useful treat or prevent cachexia in a subject in need thereof. The present invention also relates to the use of a selective melanocortin 4 receptor antagonist of the present invention in the preparation of a medicament useful treat or prevent anorexia, wasting, frailty, sarcopenia, or weight loss in a subject in need thereof.

Yet another aspect of the present invention relates to the use of a therapeutically effective amount of a melanocortin-4 receptor agonist of formula I, or a pharmaceutically acceptable salt thereof, and a therapeutically effective amount of an agent selected from the group consisting of an insulin sensitizer, an insulin mimetic, a sulfonylurea, an α-glucosidase inhibitor, a HMG-CoA reductase inhibitor, a serotonergic agent, a β3-adrenoreceptor agonist, a neuropeptide Y1 antagonist, a neuropeptide Y2 agonist, a neuropeptide Y5 antagonist, a pancreatic lipase inhibitor, a cannabinoid CB₁ receptor antagonist or inverse agonist, a melanin-concentrating hormone receptor antagonist, a bombesin receptor subtype 3 agonist, a ghrelin receptor antagonist, and a NK-1 antagonist, or a pharmaceutically acceptable salt thereof, for the manufacture of a medicament useful for the treatment, control, or prevention of obesity, diabetes or an obesity-related disorder in a subject in need of such treatment. Yet another aspect of the present invention relates to the use of a therapeutically effective amount of a melanocortin-4 receptor agonist of formula I, and pharmaceutically acceptable salts and esters thereof, and a therapeutically effective amount of an agent selected from the group consisting of an insulin sensitizer, an insulin mimetic, a sulfonylurea, an α-glucosidase inhibitor, a HMG-CoA reductase inhibitor, a serotonergic agent, a β3-adrenoreceptor agonist, a neuropeptide Y1 antagonist, a neuropeptide Y2 agonist, a neuropeptide Y5 antagonist, a pancreatic lipase inhibitor, a cannabinoid CB₁ receptor antagonist or inverse agonist, a melanin-concentrating hormone receptor antagonist, a bombesin receptor subtype 3 agonist, a ghrelin receptor antagonist, and a NK-1 antagonist, or a pharmaceutically acceptable salt thereof, for the manufacture of a medicament for treatment or prevention of obesity, diabetes or an obesity-related disorder which comprises an effective amount of a melanocortin-4 receptor agonist of formula I and an effective amount of the agent, together or separately. Yet another aspect of the present invention relates to a product containing a therapeutically effective amount of a melanocortin-4 receptor agonist of formula I, or a pharmaceutically acceptable salt thereof; and a therapeutically effective amount of an agent selected from the group consisting of an insulin sensitizer, an insulin mimetic, a sulfonylurea, an α-glucosidase inhibitor, a HMG-CoA reductase inhibitor, a serotonergic agent, a β3-adrenoreceptor agonist, a neuropeptide Y1 antagonist, a neuropeptide Y2 agonist, a neuropeptide Y5 antagonist, a pancreatic lipase inhibitor, a cannabinoid CB₁ receptor antagonist or inverse agonist, a melanin-concentrating hormone receptor antagonist, a bombesin receptor subtype 3 agonist, a ghrelin receptor antagonist, and a NK-1 antagonist, or a pharmaceutically acceptable salt thereof, as a combined preparation for simultaneous, separate or sequential use in obesity, diabetes, or an obesity-related disorder.

Yet another aspect of the present invention relates to the use of a therapeutically effective amount of a melanocortin-4 receptor agonist of formula I, or a pharmaceutically acceptable salt thereof, and a therapeutically effective amount of an agent selected from the group consisting of: a type V cyclic-GMP-selective phosphodiesterase inhibitor, an α2-adrenergic receptor antagonist, and a dopaminergic agent, or a pharmaceutically acceptable salt thereof, for the manufacture of a medicament useful for the treatment, control, or prevention of male erectile dysfunction in a subject in need of such treatment. Yet another aspect of the present invention relates to the use of a therapeutically effective amount of a melanocortin-4 receptor agonist of formula I, or a pharmaceutically acceptable salt thereof; and a therapeutically effective amount of an agent selected from the group consisting of a type V cyclic-GMP-selective phosphodiesterase inhibitor, an α2-adrenergic receptor antagonist, and a dopaminergic agent, and pharmaceutically acceptable salts and esters thereof; for the manufacture of a medicament for treatment or prevention of male erectile dysfunction which comprises an effective amount of a compound of formula I and an effective amount of the agent, together or separately. Yet another aspect of the present invention relates to a product containing a therapeutically effective amount of a melanocortin-4 receptor agonist of formula I, or a pharmaceutically acceptable salt thereof; and a therapeutically effective amount of an agent selected from the group consisting of a type V cyclic-GMP-selective phosphodiesterase inhibitor, an α2-adrenergic receptor antagonist, and a dopaminergic agent, and pharmaceutically acceptable salts and esters thereof; as a combined preparation for simultaneous, separate or sequential use in male erectile dysfunction.

Melanocortin receptor agonist compounds can be provided in kit. Such a kit typically contains an active compound in dosage forms for administration. A dosage form contains a sufficient amount of active compound such that a beneficial effect can be obtained when administered to a patient during regular intervals, such as 1, 2, 3, 4, 5 or 6 times a day, during the course of 1 or more days. Preferably, a kit contains instructions indicating the use of the dosage form for weight reduction (e.g., to treat obesity) and the amount of dosage form to be taken over a specified time period.

Throughout the instant application, the following terms have the indicated meanings:

The term “alkyl”, as well as other groups having the prefix “alk”, such as alkoxy, alkanoyl, means carbon chains of the designated length which may be in a straight or branched configuration, or combinations thereof. The term alkyl also includes methylene groups which are designated as (CH₂) herein. Examples of alkyl groups include methyl, ethyl, n-propyl, isopropyl, n-butyl, 1-methylpropyl, 2-methylpropyl, tert-butyl, n-pentyl, 1-methylbutyl, 2-methylbutyl, 3-methylbutyl, 1,2-dimethylpropyl, 1,1-dimethylpropyl, 2,2-dimethylpropyl, n-hexyl, 1-methylpentyl, 2-methylpentyl, 3-methylpentyl, 4-methylpentyl, 1-ethylbutyl, 2-ethylbutyl, 3-ethylbutyl, 1,1-dimethyl butyl, 1,2-dimethylbutyl, 1,3-dimethylbutyl, 2,2-dimethylbutyl, 2,3-dimethylbutyl, 3,3-dimethyl butyl, n-heptyl, 1-methylhexyl, 2-methylhexyl, 3-methylhexyl, 4-methylhexyl, 5-methylhexyl, 1-ethylpentyl, 2-ethylpentyl, 3-ethylpentyl, 4-ethylpentyl, 1-propylbutyl, 2-propylbutyl, 3-propylbutyl, 1,1-dimethylpentyl, 1,2-dimethylpentyl, 1,3-dimethylpentyl, 1,4-dimethylpentyl, 2,2-dimethylpentyl, 2,3-dimethylpentyl. 2,4-dimethylpentyl, 3,3-dimethylpentyl, 3,4-dimethylpentyl, 4,4-dimethylpentyl, 1-methyl-1-ethylbutyl, 1-methyl-2-ethylbutyl, 2-methyl-2-ethylbutyl, 1-ethyl-2-methylbutyl, 1-ethyl-3-methylbutyl, 1,1-diethylpropyl, n-octyl, n-nonyl, and the like.

The term “alkenyl” means carbon chains which contain at least one carbon-carbon double bond, and which may be linear or branched or combinations thereof. Examples of alkenyl include vinyl, alkyl, isopropenyl, pentenyl, hexenyl, heptenyl, 1-propenyl, 2-butenyl, 2-methyl-2-butenyl, and the like.

The term “alkynyl” means carbon chains which contain at least one carbon-carbon triple bond, and which may be linear or branched or combinations thereof. Examples of alkynyl include ethynyl, propargyl, 3-methyl-1-pentynyl, 2-heptynyl and the like.

The term “halogen” is intended to include the halogen atoms fluorine, chlorine, bromine and iodine.

The term “C₁₋₄ alkyliminoyl” means C₁₋₃alkylC(═NH)—.

The term “aryl” includes phenyl and naphthyl.

The term “heteroaryl” includes monocyclic aromatic rings, and bicyclic ring systems with at least one aromatic ring, which contain from 1 to 4 heteroatoms selected from nitrogen, oxygen, sulfur, sulfone, and sulfoxide. Examples thereof include, but are not limited to, pyridinyl, furyl, thienyl, pyrrolyl, oxazolyl, thiazolyl, triazolyl, triazinyl, tetrazolyl, thiadiazolyl, imidazolyl, isoxazolyl, isothiazolyl, oxadiazolyl, pyrazolyl, pyrimidinyl, pyrazinyl, pyridazinyl, quinolyl, isoquinolyl, benzimidazolyl, benzofuryl, benzothienyl, indolyl, benzthiazolyl, benzoxazolyl, and the like. In one embodiment of the present invention, heteroaryl is selected from the group consisting of pyridinyl, furyl, thienyl, pyrrolyl, oxazolyl, thiazolyl, triazolyl, triazinyl, tetrazolyl, thiadiazolyl, imidazolyl, pyrazolyl, isoxazolyl, isothiazolyl, oxathiazolyl, pyrimidinyl, pyrazinyl, pyridazinyl, quinolyl, isoquinolyl, benzimidazolyl, benzofuryl, benzothienyl, indolyl, benzthiazolyl, and benzoxazolyl.

Bicyclic heteroaromatic rings include, but are not limited to, benzothiadiazole, indole, benzothiophene, benzofuran, benzimidazole, benzisoxazole, benzothiazole, quinoline, quinazoline, benzotriazole, benzoxazole, isoquinoline, purine, furopyridine, thienopyridine, benzisodiazole, triazolopyrimidine, and 5,6,7,8-tetrahydroquinoline.

The term “cycloalkyl” includes mono- or bicyclic non-aromatic rings containing only carbon atoms. Examples of cycloalkyl include, but are not limited to, cyclopropyl, cyclobutyl, cyclopentyl, cyclohexyl, cycloheptyl.

The term “heterocycloalkyl” includes 2 to 9 carbon mono- or bicyclic non-aromatic rings in which each ring may contain one to four heteroatoms selected from nitrogen, oxygen, sulfur, sulfone, and sulfoxide. Substitution on the heterocycloalkyl ring includes mono- or di-substitution on any carbon and/or monosubstitution on any nitrogen of the heterocycloalkyl ring. Examples of heterocycloalkyls include, but are not limited to, azetidine, piperidine, morpholine, thiamorpholine, tetrahydropyran, thiatetrahydropyran, pyrrolidine, imidazolidine, tetrahydrofuran, piperazine, 1-thia-4-aza-cyclohexane and 1,3 oxazolidine.

Certain of the above defined terms may occur more than once in the above formula and upon such occurrence each term shall be defined independently of the other; thus for example, NR⁴R⁴ may represent NH₂, NHCH₃, N(CH₃)CH₂CH₃, and the like.

The term “subject” means a mammal. One embodiment of the term “mammal” is a “human,” said human being either male or female. The instant compounds are also useful for treating or preventing obesity and obesity related disorders in cats and dogs. As such, the term “mammal” includes companion animals such as cats and dogs. The term “mammal in need thereof” refers to a mammal who is in need of treatment or prophylaxis as determined by a researcher, veterinarian, medical doctor or other clinician.

The term “composition”, as in pharmaceutical composition, is intended to encompass a product comprising the active ingredient(s), and the inert ingredient(s) that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present invention encompass any composition made by admixing a compound of the present invention and a pharmaceutically acceptable carrier.

By a melanocortin receptor “agonist” is meant an endogenous or drug substance or compound that can interact with a melanocortin receptor and initiate a pharmacological or biochemical response characteristic of melanocortin receptor activation. By a melanocortin receptor “antagonist” is meant a drug or a compound that inhibits the melanocortin receptor-associated responses induced by an agonist. The “agonistic” and “antagonistic” properties of the compounds of the present invention were measured in the functional assay described below. The functional assay discriminates a melanocortin receptor agonist from a melanocortin receptor antagonist.

By “binding affinity” is meant the ability of a compound/drug to bind to its biological target, in the present instance, the ability of a compound of structural formula Ito bind to a melanocortin receptor. Binding affinities for the compounds of the present invention were measured in the binding assay described below and are expressed as IC₅₀'s.

“Efficacy” describes the relative intensity of response which different agonists produce even when they occupy the same number of receptors and with the same affinity. Efficacy is the property that describes the magnitude of response. Properties of compounds can be categorized into two groups, those which cause them to associate with the receptors (binding affinity) and those that produce a stimulus (efficacy). The term “efficacy” is used to characterize the level of maximal responses induced by agonists. Not all agonists of a receptor are capable of inducing identical levels of maximal responses. Maximal response depends on the efficiency of receptor coupling, that is, from the cascade of events, which, from the binding of the drug to the receptor, leads to the desired biological effect.

The functional activities expressed as EC₅₀'s and the “agonist efficacy” for the compounds of the present invention at a particular concentration were measured in the functional assay described below.

Compounds of structural formula I contain one or more asymmetric centers and can thus occur as racemates and racemic mixtures, single enantiomers, diastereomeric mixtures and individual diastereomers. The present invention is meant to comprehend all such isomeric forms of the compounds of structural formula I, including the E and Z geometric isomers of olefinic double bonds. Some of the compounds described herein may exist as tautomers such as keto-enol tautomers. The individual tautomers as well as mixtures thereof are encompassed within the compounds of structural formula I.

Compounds of structural formula I may be separated into their individual diastereoisomers by, for example, fractional crystallization from a suitable solvent, for example methanol or ethyl acetate or a mixture thereof, or via chiral chromatography using an optically active stationary phase. Absolute stereochemistry may be determined by X-ray crystallography of crystalline products or crystalline intermediates which are derivatized, if necessary, with a reagent containing an asymmetric center of known absolute configuration.

Alternatively, any stereoisomer of a compound of the general formula I, IIa and IIb may be obtained by stereospecific synthesis using optically pure starting materials or reagents of known absolute configuration.

It will be understood that the compounds of the present invention include hydrates, solvates, polymorphs, crystalline, hydrated crystalline and amorphous forms of the compounds of the present invention, and pharmaceutically acceptable salts thereof.

The term “pharmaceutically acceptable salts” refers to salts prepared from pharmaceutically acceptable non-toxic bases or acids including inorganic or organic bases and inorganic or organic acids. Salts derived from inorganic bases include aluminum, ammonium, calcium, copper, ferric, ferrous, lithium, magnesium, manganic salts, manganous, potassium, sodium, zinc, and the like. Particularly preferred are the ammonium, calcium, lithium, magnesium, potassium, and sodium salts. Salts derived from pharmaceutically acceptable organic non-toxic bases include salts of primary, secondary, and tertiary amines, substituted amines including naturally occurring substituted amines, cyclic amines, and basic ion exchange resins, such as arginine, betaine, caffeine, choline, N,N′-dibenzylethylenediamine, diethylamine, 2-diethylaminoethanol, 2-dimethylaminoethanol, ethanolamine, ethylenediamine, N-ethyl-morpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine, and the like.

When the compound of the present invention is basic, salts may be prepared from pharmaceutically acceptable non-toxic acids, including inorganic and organic acids. Such acids include acetic, benzenesulfonic, benzoic, camphorsulfonic, citric, ethanesulfonic, formic, fumaric, gluconic, glutamic, hydrobromic, hydrochloric, isethionic, lactic, maleic, malic, mandelic, methanesulfonic, malonic, mucic, nitric, pamoic, pantothenic, phosphoric, propionic, succinic, sulfuric, tartaric, p-toluenesulfonic acid, trifluoroacetic acid, and the like. Particularly preferred are citric, fumaric, hydrobromic, hydrochloric, maleic, phosphoric, sulfuric, and tartaric acids.

It will be understood that, as used herein, references to the compounds of Formula I are meant to also include the pharmaceutically acceptable salts, such as the hydrochloride salts.

Compounds of formula I are melanocortin receptor ligands and as such are useful in the treatment, control or prevention of diseases, disorders or conditions responsive to the modulation of one or more of the melanocortin receptors including, but are not limited to, MC-1, MC-2, MC-3, MC-4, or MC-5. In particular, the compounds of formula I act as melanocortin-4 receptor agonists and antagonists useful in the treatment, control or prevention of diseases, disorders or conditions responsive to the activation or deactivation of the melanocortin-4 receptor. Such diseases, disorders or conditions include, but are not limited to, obesity (by reducing appetite, increasing metabolic rate, reducing fat intake or reducing carbohydrate craving), diabetes mellitus (by enhancing glucose tolerance, decreasing insulin resistance), hypertension, hyperlipidemia, osteoarthritis, cancer, gall bladder disease, sleep apnea, depression, anxiety, compulsion, neuroses, insomnia/sleep disorder, substance abuse, pain, male and female sexual dysfunction (including male impotence, loss of libido, female sexual arousal dysfunction, female orgasmic dysfunction, hypoactive sexual desire disorder, sexual pain disorder and male erectile dysfunction), fever, inflammation, immunomodulation, rheumatoid arthritis, skin tanning, acne and other skin disorders, neuroprotective and cognitive and memory enhancement including the treatment of Alzheimer's disease. Some agonists encompassed by formula I show highly selective affinity for the melanocortin-4 receptor (MC-4R) relative to MC-1R, MC-2R, MC-3R, and MC-5R, which makes them especially useful in the prevention and treatment of obesity, female sexual dysfunction, male sexual dysfunction including erectile dysfunction, alcoholism and nicotine addiction. Some antagonists encompassed by formula I show highly selective affinity for the melanocortin-4 receptor (MC-4R) relative to MC-1R, MC-2R, MC-3R, and MC-5R, which makes them especially useful in the prevention and treatment of cachexia, wasting and anorexia.

The compositions of the present invention are useful for the treatment or prevention of disorders associated with excessive food intake, such as obesity and obesity-related disorders. The obesity herein may be due to any cause, whether genetic or environmental.

The obesity-related disorders herein are associated with, caused by, or result from obesity. Examples of obesity-related disorders include overeating, binge eating, and bulimia, hypertension, diabetes, elevated plasma insulin concentrations and insulin resistance, dyslipidemias, hyperlipidemia, endometrial, breast, prostate and colon cancer, osteoarthritis, obstructive sleep apnea, cholelithiasis, gallstones, heart disease, abnormal heart rhythms and arrythmias, myocardial infarction, congestive heart failure, coronary heart disease, sudden death, stroke, polycystic ovary disease, craniopharyngioma, the Prader-Willi Syndrome, Frohlich's syndrome, GH-deficient subjects, normal variant short stature, Turner's syndrome, and other pathological conditions showing reduced metabolic activity or a decrease in resting energy expenditure as a percentage of total fat-free mass, e.g, children with acute lymphoblastic leukemia. Further examples of obesity-related disorders are metabolic syndrome, insulin resistance syndrome, sexual and reproductive dysfunction, such as infertility, hypogonadism in males and hirsutism in females, gastrointestinal motility disorders, such as obesity-related gastro-esophageal reflux, respiratory disorders, such as obesity-hypoventilation syndrome (Pickwickian syndrome), cardiovascular disorders, inflammation, such as systemic inflammation of the vasculature, arteriosclerosis, hypercholesterolemia, hyperuricaemia, lower back pain, gallbladder disease, gout, and kidney cancer, nicotine addiction, substance addiction and alcoholism. The compositions of the present invention are also useful for reducing the risk of secondary outcomes of obesity, such as reducing the risk of left ventricular hypertrophy.

The term “metabolic syndrome”, also known as syndrome X, is defined in the Third Report of the National Cholesterol Education Program Expert Panel on Detection, Evaluation and Treatment of High Blood Cholesterol in Adults (ATP-III). E. S. Ford et al., JAMA, vol. 287 (3), Jan. 16, 2002, pp 356-359. Briefly, a person is defined as having metabolic syndrome if the person has three or more of the following symptoms: abdominal obesity, hypertriglyceridemia, low HDL cholesterol, high blood pressure, and high fasting plasma glucose. The criteria for these are defined in ATP-III.

The term “diabetes,” as used herein, includes both insulin-dependent diabetes mellitus (i.e., IDDM, also known as type I diabetes) and non-insulin-dependent diabetes mellitus (i.e., NIDDM, also known as Type II diabetes). Type I diabetes, or insulin-dependent diabetes, is the result of an absolute deficiency of insulin, the hormone which regulates glucose utilization. Type II diabetes, or insulin-independent diabetes (i.e., non-insulin-dependent diabetes mellitus), often occurs in the face of normal, or even elevated levels of insulin and appears to be the result of the inability of tissues to respond appropriately to insulin. Most of the Type II diabetics are also obese. The compositions of the present invention are useful for treating both Type I and Type II diabetes. The compositions are especially effective for treating Type II diabetes. The compounds or combinations of the present invention are also useful for treating and/or preventing gestational diabetes mellitus.

Treatment of diabetes mellitus refers to the administration of a compound or combination of the present invention to treat diabetes. One outcome of treatment may be decreasing the glucose level in a subject with elevated glucose levels. Another outcome of treatment may be improving glycemic control. Another outcome of treatment may be decreasing insulin levels in a subject with elevated insulin levels. Another outcome of treatment may be decreasing plasma triglycerides in a subject with elevated plasma triglycerides. Another outcome of treatment may be lowering LDL cholesterol in a subject with high LDL cholesterol levels. Another outcome of treatment may be increasing HDL cholesterol in a subject with low HDL cholesterol levels. Another outcome may be decreasing the LDL/HDL ratio in a subject in need thereof. Another outcome of treatment may be increasing insulin sensitivity. Another outcome of treatment may be enhancing glucose tolerance in a subject with glucose intolerance. Another outcome of treatment may be decreasing insulin resistance in a subject with increased insulin resistance or elevated levels of insulin. Another outcome may be decreasing triglycerides in a subject with elevated triglycerides. Yet another outcome may be improving LDL cholesterol, non-HDL cholesterol, triglyceride, HDL cholesterol or other lipid analyte profiles.

Prevention of diabetes mellitus refers to the administration of a compound or combination of the present invention to prevent the onset of diabetes in a subject at risk thereof.

“Obesity” is a condition in which there is an excess of body fat. The operational definition of obesity is based on the Body Mass Index (BMI), which is calculated as body weight per height in meters squared (kg/m²). “Obesity” refers to a condition whereby an otherwise healthy subject has a Body Mass Index (BMI) greater than or equal to 30 kg/m², or a condition whereby a subject with at least one co-morbidity has a BMI greater than or equal to 27 kg/m². An “obese subject” is an otherwise healthy subject with a Body Mass Index (BMI) greater than or equal to 30 kg/m² or a subject with at least one co-morbidity with a BMI greater than or equal to 27 kg/m². A “subject at risk of obesity” is an otherwise healthy subject with a BMI of 25 kg/m² to less than 30 kg/m² or a subject with at least one co-morbidity with a BMI of 25 kg/m² to less than 27 kg/m².

The increased risks associated with obesity occur at a lower Body Mass Index (BMI) in Asians. In Asian countries, including Japan, “obesity” refers to a condition whereby a subject with at least one obesity-induced or obesity-related co-morbidity, that requires weight reduction or that would be improved by weight reduction, has a BMI greater than or equal to 25 kg/m². In Asian countries, including Japan, an “obese subject” refers to a subject with at least one obesity-induced or obesity-related co-morbidity that requires weight reduction or that would be improved by weight reduction, with a BMI greater than or equal to 25 kg/m². In Asia-Pacific, a “subject at risk of obesity” is a subject with a BMI of greater than 23 kg/m² to less than 25 kg/m².

As used herein, the term “obesity” is meant to encompass all of the above definitions of obesity.

Obesity-induced or obesity-related co-morbidities include, but are not limited to, diabetes, non-insulin dependent diabetes mellitus-type II (2), impaired glucose tolerance, impaired fasting glucose, insulin resistance syndrome, dyslipidemia, hypertension, hyperuricacidemia, gout, coronary artery disease, myocardial infarction, angina pectoris, sleep apnea syndrome, Pickwickian syndrome, fatty liver; cerebral infarction, cerebral thrombosis, transient ischemic attack, orthopedic disorders, arthritis deformans, lumbodynia, emmeniopathy, and infertility. In particular, co-morbidities include: hypertension, hyperlipidemia, dyslipidemia, glucose intolerance, cardiovascular disease, sleep apnea, diabetes mellitus, and other obesity-related conditions.

Treatment of obesity and obesity-related disorders refers to the administration of the compounds or combinations of the present invention to reduce or maintain the body weight of an obese subject. One outcome of treatment may be reducing the body weight of an obese subject relative to that subject's body weight immediately before the administration of the compounds or combinations of the present invention. Another outcome of treatment may be preventing body weight regain of body weight previously lost as a result of diet, exercise, or pharmacotherapy. Another outcome of treatment may be decreasing the occurrence of and/or the severity of obesity-related diseases. The treatment may suitably result in a reduction in food or calorie intake by the subject, including a reduction in total food intake, or a reduction of intake of specific components of the diet such as carbohydrates or fats; and/or the inhibition of nutrient absorption; and/or the inhibition of the reduction of metabolic rate; and in weight reduction in subjects in need thereof. The treatment may also result in an alteration of metabolic rate, such as an increase in metabolic rate, rather than or in addition to an inhibition of the reduction of metabolic rate; and/or in minimization of the metabolic resistance that normally results from weight loss.

Prevention of obesity and obesity-related disorders refers to the administration of the compounds or combinations of the present invention to reduce or maintain the body weight of a subject at risk of obesity. One outcome of prevention may be reducing the body weight of a subject at risk of obesity relative to that subject's body weight immediately before the administration of the compounds or combinations of the present invention. Another outcome of prevention may be preventing body weight regain of body weight previously lost as a result of diet, exercise, or pharmacotherapy. Another outcome of prevention may be preventing obesity from occurring if the treatment is administered prior to the onset of obesity in a subject at risk of obesity. Another outcome of prevention may be decreasing the occurrence and/or severity of obesity-related disorders if the treatment is administered prior to the onset of obesity in a subject at risk of obesity. Moreover, if treatment is commenced in already obese subjects, such treatment may prevent the occurrence, progression or severity of obesity-related disorders, such as, but not limited to, arteriosclerosis, Type II diabetes, polycystic ovary disease, cardiovascular diseases, osteoarthritis, dermatological disorders, hypertension, insulin resistance, hypercholesterolemia, hypertriglyceridemia, and cholelithiasis.

“Male sexual dysfunction” includes impotence, loss of libido, and erectile dysfunction.

“Erectile dysfunction” is a disorder involving the failure of a male subject to achieve erection, ejaculation, or both. Symptoms of erectile dysfunction include an inability to achieve or maintain an erection, ejaculatory failure, premature ejaculation, or inability to achieve an orgasm. An increase in erectile dysfunction and sexual dysfunction can have numerous underlying causes, including but not limited to (1) aging, (b) an underlying physical dysfunction, such as trauma, surgery, and peripheral vascular disease, and (3) side-effects resulting from drug treatment, depression, and other CNS disorders.

Treatment of male sexual dysfunction refers to the administration of a compound or combination of the present invention to treat impotence and/or loss of libido, and/or erectile dysfunction in a male subject in need thereof. One outcome of treatment may be a decrease in impotence. Another outcome of treatment may be an increase in libido. Yet another outcome of treatment may be a decrease in the magnitude or frequency of erectile dysfunction. Treatment of male erectile dysfunction refers to the administration of a compound or combination of the present invention to treat one or more of the symptoms of male erectile dysfunction in a male subject in need thereof. One outcome of treatment may be increasing the ability to achieve an erection. Another outcome of treatment may be increasing the ability to maintain an erection. Another outcome of treatment may be reducing ejaculatory failure. Another outcome of treatment may be decreasing premature ejaculation. Yet another outcome of treatment may be increasing the ability to achieve an orgasm. Prevention of male sexual dysfunction and male erectile dysfunction refers to the administration of the compounds or combinations of the present invention to prevent the symptoms of sexual dysfunction and erectile dysfunction in a male subject at risk thereof.

“Female sexual dysfunction” can be seen as resulting from multiple components including dysfunction in desire, sexual arousal, sexual receptivity, and orgasm related to disturbances in the clitoris, vagina, periurethral glans, and other trigger points of sexual function. In particular, anatomic and functional modification of such trigger points may diminish the orgasmic potential in breast cancer and gynecologic cancer patients. Treatment of female sexual dysfunction with an MC-4 receptor agonist can result in improved blood flow, improved lubrication, improved sensation, facilitation of reaching orgasm, reduction in the refractory, period between orgasms, and improvements in arousal and desire. In a broader sense, “female sexual dysfunction” also incorporates sexual pain, premature labor, and dysmenorrhea.

The compositions of the present invention are useful for the treatment or prevention of disorders associated with excessive food intake, such as obesity and obesity-related disorders.

“Cachexia” is a wasting disorder that is characterized by weight loss, loss of muscle protein, loss of lean body mass, anorexia, and weakness, and is typically associated with chronic diseases, including cancer cachexia and cachexia associated with AIDS, chronic obstructive pulmonary disease, rheumatiod arthritis, tuberculosis and Crohn's disease. Cancer cachexia is a syndrome of progressive weight loss, anorexia, and persistent erosion of the body in response to a malignant growth; cachexia may be present in early stages of tumor growth before any signs or symptoms of malignancy.

Treatment of cachexia refers to the administration of a compound or combination of the present invention to treat one or more of the symptoms of cachexia in a subject in need thereof.

Prevention of cachexia refers to the administration of the compounds or combinations of the present invention to prevent the symptoms of cachexia or wasting in a subject at risk thereof, including but not limited to, a subject diagnosed with cancer.

The compositions of the present invention are useful for the treatment or prevention of nicotine addiction, substance addiction, and alcoholism, as well as nicotine addiction related disorders, substance abuse related disorders, and alcoholism related disorders.

The term “nicotine” as used herein refers to nicotine contained in tobacco and other naturally occurring sources, as well as synthetic nicotine, and salts thereof, including but not limited to, the salicylate or bitartrate salt thereof. Nicotine addiction is a destructive pattern of nicotine use, leading to significant social occupational, or medical impairment and characterized by three or more of the following symptoms: 1) nicotine tolerance (a need for markedly increased amounts of nicotine to achieve intoxication, or markedly diminished effect with continued use of the same amount of nicotine); 2) nicotine withdrawal symptoms (sweating or rapid pulse, increased hand tremor, insomnia, nausea or vomiting, physical agitation, anxiety, transient visual, tactile, or auditory hallucinations or illusions, grand mal seizures), 3) nicotine administration to relieve or avoid withdrawal symptoms, 4) greater use than nicotine than intended, 5) unsuccessful efforts to cut down or control nicotine use, 6) persistent desire or unsuccessful efforts to cut down or control nicotine use, 7) great deal of time spent using nicotine, 8) nicotine caused reduction in social, occupational or recreational activities, and 9) continued use of nicotine despite knowledge of having a persistent or recurrent physical or psychological problem that is likely to have been worsened by nicotine use. Nicotine addiction-related disorders include, but are not limited to: cancer of the lung, mouth, pharynx, larynx, esophagus, cervix, kidney, ureter and bladder; chronic bronchitis; emphysema; asthma; heart disease, including stroke, heart attack, vascular disease, and aneurysm; premature delivery; spontaneous abortion; and infants with decreased birth weight; as well as nicotine withdrawal symptoms. “Treatment” (of nicotine addiction) refers to the administration of the compounds or combinations of the present invention to reduce or inhibit the use of nicotine by a subject. One outcome of treatment may be reducing the use of nicotine in a subject relative to the subject's nicotine use prior to treatment. Another outcome of treatment may be inhibiting the use of nicotine in a subject. Another outcome of treatment may be decreasing the severity of nicotine intake, such as decreasing the amount of nicotine consumed, in a subject. “Prevention” (of nicotine addiction) refers to the administration of the compounds or combinations of the present invention to prevent nicotine abuse, nicotine addiction or developing a nicotine addiction-related disorder in a subject by administration prior to the start of nicotine use. One outcome of prevention may be to prevent nicotine use in a subject by administration prior to the start of nicotine use. Another outcome of prevention may be to prevent nicotine addiction in a subject. Another outcome of prevention may be to prevent the development of a nicotine addiction related disorder in a subject. Another outcome of prevention may be preventing nicotine use from occurring if the treatment is administered prior to the onset of nicotine use in a subject. Another outcome of prevention may be to administer the compounds or combinations of the present invention to prevent nicotine use in a subject at risk of developing nicotine addiction.

Substance addiction includes opiate addiction, cocaine addiction, marijuana addiction, and amphetamine addiction. The term “opiate” as used herein includes, but is not limited to, heroin; narcotics, such as morphine; opium; codeine; oxycodone (Oxycontin®); propoxyphene (Darvon®); hydrocodone (Vicodin®), hydromorphone (Dilaudid®); meperidine (Demerol®), and Lomotil®. The term “amphetamine(s)” as used herein includes, but is not limited to, amphetamine, dextroamphetamine, and methamphetamine. “Treatment” (of substance addiction) refers to the administration of the compounds or combinations of the present invention to reduce or inhibit the use of the substance by a subject. One outcome of treatment may be reducing the use of the substance in a subject relative to the subject's substance use prior to treatment. Another outcome of treatment may be inhibiting the use of the substance in a subject. Another outcome of treatment may be decreasing the occurrence of substance intake in a subject. Another outcome of treatment may be decreasing the severity of substance intake, such as decreasing the amount of the substance consumed, in a subject. Another outcome of treatment may be to administer the compounds or combinations of the present invention to reduce or inhibit the consumption of the substance in a subject in need thereof. “Prevention” (of substance addiction) refers to the administration of the compounds or combinations of the present invention to prevent substance addiction or developing a substance addiction-related disorder in a subject. One outcome of prevention may be to prevent substance use in a subject by administration prior to the start of substance use. Another outcome of prevention may be to prevent substance addiction in a subject. Another outcome of prevention may be to prevent the development of a substance addiction related disorder in a subject. Another outcome of prevention may be preventing substance use from occurring if the treatment is administered prior to the onset of substance use in a subject.

The compounds of the present invention are useful to inhibit or reduce voluntary alcohol consumption, and for the treatment or prevention of alcoholism, alcohol abuse, and alcohol-related disorders. Alcoholism is a disease that is characterized by abnormal alcohol seeking behavior that leads to impaired control over drinking, and may include some or all of the following symptoms: narrowing of drinking repertoire (drinking only one brand or type of alcoholic beverage); craving (a strong need or urge to drink), loss of control (not being able to stop drinking once drinking has begun), drink seeking behavior (attending only social events that include drinking); physical dependence (withdrawal symptoms, such as nausea, sweating, shakiness, and anxiety after cessation of drinking), drinking to relieve or avoid withdrawal symptoms; and tolerance (the need to drink greater amounts of alcohol to achieve previous effects); subjective awareness of the compulsion to drink or craving for alcohol; and relapse (a return to drinking after a period of abstinence). Alcohol related disorders include, but are not limited to: liver disease, such as hepatitis, inflammation of the liver, and alcoholic cirrhosis; heart disease; high blood pressure; stroke; certain forms of cancer, such as esophageal, mouth, throat, voice box, breast, colon and rectal cancer; pancreatitis; alcoholic dementia, Wernicke-Korsakoff syndrome, brain damage, slow bone healing; impaired wound healing; diminished immune defenses; and death. “Treatment” (of alcoholism) refers to the administration of the compounds or combinations of the present invention to reduce or inhibit the consumption of alcohol in a subject. One outcome of treatment may be reducing the consumption of alcohol in a subject relative to the subject's alcohol consumption prior to treatment. Another outcome of treatment may be inhibiting consumption of alcohol in a subject. Another outcome of treatment may be decreasing the occurrence of alcohol intake in a subject. Another outcome of treatment may be decreasing the severity of alcohol intake, such as decreasing the amount of alcohol consumed, in a subject. Another outcome of treatment may be to administer the compounds or combinations of the present invention to reduce or inhibit the consumption of alcohol in a subject in need thereof. “Prevention” (of alcoholism) refers to the administration of the compounds or combinations of the present invention to prevent alcohol intake, alcohol consumption, alcohol abuse, alcoholism or developing an alcohol-related disorder in a subject. One outcome of prevention may be to prevent alcohol intake in a subject by administration prior to the start of alcohol consumption. Another outcome of prevention may be to prevent alcoholism in a subject. Another outcome of prevention may be to administer the compounds or combinations of the present invention to prevent alcohol intake in a subject at risk of alcoholism or developing an alcohol-related disorder in a subject. Moreover, if treatment is commenced in a subject already consuming alcohol, such treatment may prevent the occurrence, progression or severity of alcohol-related disorders.

The terms “administration of” and or “administering” a compound should be understood to mean providing a compound of the invention or a prodrug of a compound of the invention to a subject in need of treatment. The administration of the compounds of the present invention in order to practice the present methods of therapy is carried out by administering a therapeutically effective amount of the compound to a subject in need of such treatment or prophylaxis. The need for a prophylactic administration according to the methods of the present invention is determined via the use of well known risk factors.

The term “therapeutically effective amount” as used herein means the amount of the active compound that will elicit the biological or medical response in a tissue, system, subject, mammal, or human that is being sought by the researcher, veterinarian, medical doctor or other clinician, which includes alleviation of the symptoms of the disorder being treated. The novel methods of treatment of this invention are for disorders known to those skilled in the art. The term “prophylactically effective amount” as used herein means the amount of the active compound that will elicit the biological or medical response in a tissue, system, subject, mammal, or human that is being sought by the researcher, veterinarian, medical doctor or other clinician, to prevent the onset of the disorder in subjects as risk for obesity or the disorder. The therapeutically or prophylactically effective amount, or dosage, of an individual compound is determined, in the final analysis, by the physician in charge of the case, but depends on factors such as the exact disease to be treated, the severity of the disease and other diseases or conditions from which the patient suffers, the chosen route of administration, other drugs and treatments which the patient may concomitantly require, and other factors in the physician's judgement.

### Administration and Dose Ranges

Any suitable route of administration may be employed for providing a subject or mammal, especially a human with an effective dosage of a compound of the present invention. For example, oral, rectal, topical, parenteral, ocular, pulmonary, nasal, and the like may be employed. Dosage forms include tablets, troches, dispersions, suspensions, solutions, capsules, creams, ointments, aerosols, and the like. Preferably compounds of Formula I are administered orally or topically.

The effective dosage of active ingredient employed may vary depending on the particular compound employed, the mode of administration, the condition being treated and the severity of the condition being treated. Such dosage may be ascertained readily by a person skilled in the art.

When treating obesity, in conjunction with diabetes and/or hyperglycemia, or alone, generally satisfactory results are obtained when the compounds of formula I are administered at a daily dosage of from about 0.001 milligram to about 50 milligrams per kilogram of animal body weight, preferably given in a single dose or in divided doses two to six times a day, or in sustained release form. In the case of a 70 kg adult human, the total daily dose will generally be from about 0.07 milligrams to about 3500 milligrams. This dosage regimen may be adjusted to provide the optimal therapeutic response.

When treating diabetes mellitus and/or hyperglycemia, as well as other diseases or disorders for which compounds of formula I are useful, generally satisfactory results are obtained when the compounds of the present invention are administered at a daily dosage of from about 0.001 milligram to about 50 milligram per kilogram of animal body weight, preferably given in a single dose or in divided doses two to six times a day, or in sustained release form. In the case of a 70 kg adult human, the total daily dose will generally be from about 0.07 milligrams to about 3500 milligrams. This dosage regimen may be adjusted to provide the optimal therapeutic response.

For the treatment of sexual dysfunction compounds of formula I are given in a dose range of 0.001 milligram to about 50 milligram per kilogram of body weight, preferably as a single dose orally or as a nasal spray.

When treating cachexia or weight loss, satisfactory results are obtained when the compounds of formula I are administered at a daily dosage of from about 0.001 milligram to about 50 milligrams per kilogram of animal body weight, preferably given in a single dose or in divided doses two to six times a day, or in sustained release form. In the case of a 70 kg adult human, the total daily dose will generally be from about 0.07 milligrams to about 3500 milligrams. This dosage regimen may be adjusted to provide the optimal therapeutic response.

In the case where an oral composition is employed, a suitable dosage range is, e.g. from about 0.01 mg to about 1500 mg of a compound of Formula I per day, preferably from about 0.1 mg to about 600 mg per day, more preferably from about 0.1 mg to about 100 mg per day. For oral administration, the compositions are preferably provided in the form of tablets containing from 0.01 to 1,000 mg, preferably 0.01, 0.05, 0.1, 0.5, 1, 2.5, 5, 10, 15, 20, 25, 30, 40, 50, 100, 250, 500, 600, 750, 1000, 1250 or 1500 milligrams of the active ingredient for the symptomatic adjustment of the dosage to the patient to be treated.

For use where a composition for intranasal administration is employed, intranasal formulations for intranasal administration comprising 0.001-10% by weight solutions or suspensions of the compounds of Formula I in an acceptable intranasal formulation may be used.

For use where a composition for intravenous administration is employed, a suitable dosage range is from about 0.001 mg to about 50 mg, preferably from 0.01 mg to about 50 mg, more preferably 0.1 mg to 10 mg, of a compound of Formula I per kg of body weight per day. This dosage regimen may be adjusted to provide the optimal therapeutic response. It may be necessary to use dosages outside these limits in some cases.

For the treatment of diseases of the eye, ophthalmic preparations for ocular administration comprising 0.001-1% by weight solutions or suspensions of the compounds of Formula I in an acceptable ophthalmic formulation may be used.

The magnitude of prophylactic or therapeutic dosage of the compounds of the present invention will, of course, vary depending on the particular compound employed, the mode of administration, the condition being treated and the severity of the condition being treated. It will also vary according to the age, weight and response of the individual patient. Such dosage may be ascertained readily by a person skilled in the art.

Compounds of Formula I may be used in combination with other drugs that are used in the treatment/prevention/suppression or amelioration of the diseases or conditions for which compounds of Formula I are useful. Such other drugs may be administered, by a route and in an amount commonly used therefor, contemporaneously or sequentially with a compound of Formula I. When a compound of Formula I is used contemporaneously with one or more other drugs, a pharmaceutical composition containing such other drugs in addition to the compound of Formula I is preferred. Accordingly, the pharmaceutical compositions of the present invention include those that also contain one or more other active ingredients, in addition to a compound of Formula I.

Examples of other active ingredients that may be combined with a compound of Formula I for the treatment or prevention of obesity and/or diabetes, either administered separately or in the same pharmaceutical compositions, include, but are not limited to:

(a) insulin sensitizers including (i) PPARγ antagonists such as glitazones (e.g. ciglitazone; darglitazone; englitazone; isaglitazone (MCC-555); pioglitazone; rosiglitazone; troglitazone; tularik; BRL49653; CLX-0921; 5-BTZD), GW-0207, LG-100641, and LY-300512, and the like), and compounds disclosed in WO 97/10813, WO 97/27857, WO 97/28115, WO 97/28137, and WO 97/27847; (iii) biguanides such as metformin and phenformin;

(b) insulin or insulin mimetics, such as biota, LP-100, novarapid, insulin detemir, insulin lispro, insulin glargine, insulin zinc suspension (lente and ultralente); Lys-Pro insulin, GLP-1 (73-7) (insulintropin); and GLP-1 (7-36)-NH₂);

(c) sulfonylureas, such as acetohexamide; chlorpropamide; diabinese; glibenclamide; glipizide; glyburide; glimepiride; gliclazide; glipentide; gliquidone; glisolamide; tolazamide; and tolbutamide;

(d) α-glucosidase inhibitors, such as acarbose, adiposine; camiglibose; emiglitate; miglitol; voglibose; pradimicin-Q; salbostatin; CKD-711; MDL-25,637; MDL-73,945; and MOR 14, and the like;

(e) cholesterol lowering agents such as (i) HMG-CoA reductase inhibitors (atorvastatin, itavastatin, fluvastatin, lovastatin, pravastatin, rivastatin, rosuvastatin, simvastatin, and other statins), (ii) bile acid absorbers/sequestrants, such as cholestyramine, colestipol, dialkylaminoalkyl derivatives of a cross-linked dextran; Colestid®; LoCholest®, and the like, (ii) nicotinyl alcohol, nicotinic acid or a salt thereof, (iii) proliferator-activater receptor α agonists such as fenofibric acid derivatives (gemfibrozil, clofibrate, fenofibrate and benzafibrate), (iv) inhibitors of cholesterol absorption such as stanol esters, beta-sitosterol, sterol glycosides such as tiqueside; and azetidinones such as ezetimibe, and the like, and (acyl CoA:cholesterol acyltransferase (ACAT)) inhibitors such as avasimibe, and melinamide, (v) anti-oxidants, such as probucol, (vi) vitamin E, and (vii) thyromimetics;

(f) PPARα agonists such as beclofibrate, benzafibrate, ciprofibrate, clofibrate, etofibrate, fenofibrate, and gemfibrozil; and other fibric acid derivatives, such as Atromid®, Lopid® and Tricor®, and the like, and PPARα agonists as described in WO 97/36579 by Glaxo;

(g) PPARδ agonists, such as those disclosed in WO97/28149;

(h) PPAR α/δ agonists, such as muraglitazar, and the compounds disclosed in U.S. Pat. No. 6,414,002;

(i) smoking cessation agents, such as a nicotine agonist or a partial nicotine agonist such as varenicline, or a monoamine oxidase inhibitor (MAOI), or another active ingredient demonstrating efficacy in aiding cessation of tobacco consumption; for example, an antidepressant such as bupropion, doxepine, ornortriptyline; or an anxiolytic such as buspirone or clonidine; and

(j) anti-obesity agents, such as (1) growth hormone secretagogues, growth hormone secretagogue receptor agonists/antagonists, such as NN703, hexarelm, MK-0677, SM-130686, CP-424,391, L-692,429, and L-163,255, and such as those disclosed in U.S. Pat. Nos. 5,536,716, and 6,358,951, U.S. Patent Application Nos. 2002/049196 and 2002/022637, and PCT Application Nos. WO 01/56592 and WO 02/32888; (2) protein tyrosine phosphatase-1B (PTP-1B) inhibitors; (3) cannabinoid receptor ligands, such as cannabinoid CB₁ receptor antagonists or inverse agonists, such as rimonabant (Sanofi Synthelabo), AMT-251, and SR-14778 and SR 141716A (Sanofi Synthelabo), SLV-319 (Solvay), BAY 65-2520 (Bayer), and those disclosed in U.S. Pat. Nos. 5,532,237, 4,973,587, 5,013,837, 5,081,122, 5,112,820, 5,292,736, 5,624,941, 6,028,084, PCT Application Nos. WO 96/33159, WO 98/33765, WO98/43636, WO98/43635, WO 01/09120, WO98/31227, WO98/41519, WO98/37061, WO00/10967, WO00/10968, WO97/29079, WO99/02499, WO 01/58869, WO 01/64632, WO 01/64633, WO 01/64634, WO02/076949, WO 03/007887, WO 04/048317, and WO 05/000809; and EPO Application No. EP-658546, EP-656354, EP-576357; (4) anti-obesity serotonergic agents, such as fenfluramine, dexfenfluramine, phentermine, and sibutramine; (5) β3-adrenoreceptor agonists, such as AD9677/TAK677 (Dainippon/Takeda), CL-316,243, SB 418790, BRL-37344, L-796568, BMS-196085, BRL-35135A, CGP12177A, BTA-243, Trecadrine, Zeneca D7114, SR 59119A, and such as those disclosed in U.S. Pat. No. 5,705,515, and U.S. Pat. No. 5,451,677 and PCT Patent Publications WO94/18161, WO95/29159, WO97/46556, WO98/04526 and WO98/32753, WO 01/74782, and WO 02/32897; (6) pancreatic lipase inhibitors, such as orlistat (Xenical®), Triton WR1339, RHC80267, lipstatin, tetrahydrolipstatin, teasaponin, diethylumbelliferyl phosphate, and those disclosed in PCT Application No. WO 01/77094; (7) neuropeptide Y1 antagonists, such as BIBP3226, J-115814, BIBO 3304, LY-357897, CP-671906, GI-264879A, and those disclosed in U.S. Pat. No. 6,001,836, and PCT Patent Publication Nos. WO 96/14307, WO 01/23387, WO 99/51600, WO 01/85690, WO 01/85098, WO 01/85173, and WO 01/89528; (8) neuropeptide Y5 antagonists, such as GW-569180A, GW-594884A, GW-587081×, GW-548118×, FR226928, FR 240662, FR252384, 1229U91, GI-264879A, CGP71683A, LY-377897, PD-160170, SR-120562A, SR-120819A and JCF-104, and those disclosed in U.S. Pat. Nos. 6,057,335; 6,043,246; 6,140,354; 6,166,038; 6,180,653; 6,191,160; 6,313,298; 6,335,345; 6,337,332; 6,326,375; 6,329,395; 6,340,683; 6,388,077; 6,462,053; 6,649,624; and 6,723,847, hereby incorporated by reference in their entirety; European Patent Nos. EP-01010691, and EP-01044970; and PCT International Patent Publication Nos. WO 97/19682, WO 97/20820, WO 97/20821, WO 97/20822, WO 97/20823, WO 98/24768; WO 98/25907; WO 98/25908; WO 98/27063, WO 98/47505; WO 98/40356; WO 99/15516; WO 99/27965; WO 00/64880, WO 00/68197, WO 00/69849, WO 01/09120, WO 01/14376; WO 01/85714, WO 01/85730, WO 01/07409, WO 01/02379, WO 01/02379, WO 01/23388, WO 01/23389, WO 01/44201, WO 01/62737, WO 01/62738, WO 01/09120, WO 02/22592, WO 0248152, and WO 02/49648; WO 02/094825; WO 03/014083; WO 03/10191; WO 03/092889; WO 04/002986; and WO 04/031175; (9) melanin-concentrating hormone (MCH) receptor antagonists, such as those disclosed in WO 01/21577 and WO 01/21169; (10) melanin-concentrating hormone 1 receptor (MCH1R) antagonists, such as T-226296 (Takeda), and those disclosed in PCT Patent Application Nos. WO 01/82925, WO 01/87834, WO 02/051809, WO 02/06245, WO 02/076929, WO 02/076947, WO 02/04433, WO 02/51809, WO 02/083134, WO 02/094799, WO 03/004027, and Japanese Patent Application Nos. JP 13226269, and JP 2004-139909; (11) melanin-concentrating hormone 2 receptor (MCH2R) agonist/antagonists; (12) orexin-1 receptor antagonists, such as SB-334867-A, and those disclosed in PCT Patent Application Nos. WO 01/96302, WO 01/68609, WO 02/51232, and WO 02/51838; (13) serotonin reuptake inhibitors such as fluoxetine, paroxetine, and sertraline, and those disclosed in U.S. Pat. No. 6,365,633, and PCT Patent Application Nos. WO 01/27060 and WO 01/162341; (14) melanocortin agonists, such as Melanotan II or those described in WO 99/64002 and WO 00/74679; (15) other Mc4r (melanocortin 4 receptor) agonists, such as CH₁R86036 (Chiron), ME-10142, and ME-10145 (Melacure), CH₁R86036 (Chiron); PT-141, and PT-14 (Palatin), and those disclosed in: U.S. Pat. Nos. 6,410,548; 6,294,534; 6,350,760; 6,458,790; 6,472,398; 6,376,509; and 6,818,658; US Patent Publication No. US2002/0137664; US2003/0236262; US2004/009751; US2004/0092501; and PCT Application Nos. WO 99/64002; WO 00/74679; WO 01/70708; WO 01/70337; WO 01/74844; WO 01/91752; WO 01/991752; WO 02/15909; WO 02/059095; WO 02/059107; WO 02/059108; WO 02/059117; WO 02/067869; WO 02/068387; WO 02/068388; WO 02/067869; WO 02/11715; WO 02/12166; WO 02/12178; WO 03/007949; WO 03/009847; WO 04/024720; WO 04/078716; WO 04/078717; WO 04/087159; WO 04/089307; and WO 05/009950; (16) 5HT-2 agonists; (17) 5HT2C (serotonin receptor 2C) agonists, such as BVT933, DPCA37215, WAY161503, R-1065, and those disclosed in U.S. Pat. No. 3,914,250, and PCT Application Nos. WO 02/36596, WO 02/48124, WO 02/10169, WO 01/66548, WO 02/44152, WO 02/51844, WO 02/40456, and WO 02/40457; (18) galanin antagonists; (19) CCK agonists; (20) CCK-A (cholecystokinin-A) agonists, such as AR-R 15849, GI 181771, JMV-180, A-71378, A-71623 and SR146131, and those described in U.S. Pat. No. 5,739,106; (21) GLP-1 agonists; (22) corticotropin-releasing hormone agonists; (23) histamine receptor-3 (H3) modulators; (24) histamine receptor-3 (H3) antagonists/inverse agonists, such as hioperamide, 3-(1H-imidazol-4-yl)propyl N-(4-pentenyl)carbamate, clobenpropit, iodophenpropit, imoproxifan, GT2394 (Gliatech), and those described and disclosed in PCT Application No. WO 02/15905, and O-[3-(1H-imidazol-4-yl)propanol]-carbamates (Kiec-Kononowicz, K. et al., Pharmazie, 55:349-55 (2000)), piperidine-containing histamine H3-receptor antagonists (Lazewska, D. et al., Pharmazie, 56:927-32 (2001), benzophenone derivatives and related compounds (Sasse, A. et al., Arch. Pharm. (Weinheim) 334:45-52 (2001)), substituted N-phenylcarbamates (Reidemeister, S. et al., Pharmazie, 55:83-6 (2000)), and proxifan derivatives (Sasse, A. et al., J. Med. Chem. 43:3335-43 (2000)); (25) β-hydroxy steroid dehydrogenase-1 inhibitors (β-HSD-1); 26) PDE (phosphodiesterase) inhibitors, such as theophylline, pentoxifylline, zaprinast, sildenafil, aminone, milrinone, cilostamide, rolipram, and cilomilast; (27) phosphodiesterase-3B (PDE3B) inhibitors; (28) NE (norepinephrine) transport inhibitors, such as GW 320659, despiramine, talsupram, and nomifensine; (29) ghrelin receptor antagonists, such as those disclosed in PCT Application Nos. WO 01/87335, and WO 02/08250; (30) leptin, including recombinant human leptin (PEG-OB, Hoffman La Roche) and recombinant methionyl human leptin (Amgen); (31) leptin derivatives, such as those disclosed in U.S. Pat. Nos. 5,552,524, 5,552,523, 5,552,522, 5,521,283, and PCT International Publication Nos. WO 96/23513, WO 96/23514, WO 96/23515, WO 96/23516, WO 96/23517, WO 96/23518, WO 96/23519, and WO 96/23520; (32) BRS3 (bombesin receptor subtype 3) agonists such as [D-Phe6,beta-Ala11,Phe13,Nle14]Bn(6-14) and [D-Phe6,Phe13]Bn(6-13)propylamide, and those compounds disclosed in Pept. Sci. 2002 August; 8(8): 461-75); (33) CNTF (Ciliary neurotrophic factors), such as GI-181771 (Glaxo-SmithKline), SR146131 (Sanofi Synthelabo), butabindide, PD170,292, and PD 149164 (Pfizer); (34) CNTF derivatives, such as axokine (Regeneron), and those disclosed in PCT Application Nos. WO 94/09134, WO 98/22128, and WO 99/43813; (35) monoamine reuptake inhibitors, such as sibutramine, and those disclosed in U.S. Pat. Nos. 4,746,680, 4,806,570, and 5,436,272, U.S. Patent Publication No. 2002/0006964 and PCT Application Nos. WO 01/27068, and WO 01/62341; (36) UCP-1 (uncoupling protein-1), 2, or 3 activators, such as phytanic acid, 4-[(E)-2-(5,6,7,8-tetrahydro-5,5,8,8-tetramethyl-2-napthalenyl)-1-propenyl]benzoic acid (TTNPB), retinoic acid, and those disclosed in PCT Patent Application No. WO 99/00123; (37) thyroid hormone β agonists, such as KB-2611 (KaroBioBMS), and those disclosed in PCT Application No. WO 02/15845, and Japanese Patent Application No. JP 2000256190; (38) FAS (fatty acid synthase) inhibitors, such as Cerulenin and C75; (39) DGAT1 (diacylglycerol acyltransferase 1) inhibitors; (40) DGAT2 (diacylglycerol acyltransferase 2) inhibitors; (41) ACC2 (acetyl-CoA carboxylase-2) inhibitors; (42) glucocorticoid antagonists; (43) acyl-estrogens, such as oleoyl-estrone, disclosed in del Mar-Grasa, M. et al., Obesity Research, 9:202-9 (2001); (44) dipeptidyl peptidase IV (DP-IV) inhibitors, such as isoleucine thiazolidide, valine pyrrolidide, NVP-DPP728, LAF237, P93/01, TSL 225, TMC-2A/2B/2C, FE 999011, P9310/K364, VIP 0177, SDZ 274-444; and the compounds disclosed in U.S. Pat. No. 6,699,871, which is incorporated herein by reference; and International Patent Application Nos. WO 03/004498; WO 03/004496; EP 1 258 476; WO 02/083128; WO 02/062764; WO 03/000250; WO 03/002530; WO 03/002531; WO 03/002553; WO 03/002593; WO 03/000180; and WO 03/000181; (46) dicarboxylate transporter inhibitors; (47) glucose transporter inhibitors; (48) phosphate transporter inhibitors; (49) Metformin (Glucophage®); and (50) Topiramate (Topimax®); and (50) peptide YY, PYY 3-36, peptide YY analogs, derivatives, and fragments such as BIM-43073D, BIM-43004C (Olitvak, D. A. et al., Dig. Dis. Sci. 44(3):643-48 (1999)), and those disclosed in U.S. Pat. No. 5,026,685, U.S. Pat. No. 5,604,203, U.S. Pat. No. 5,574,010, U.S. Pat. No. 5,696,093, U.S. Pat. No. 5,936,092, U.S. Pat. No. 6,046,162, U.S. Pat. No. 6,046,167, U.S. Pat. No. 6,093,692, U.S. Pat. No. 6,225,445, U.S. Pat. No. 5,604,203, U.S. Pat. No. 4,002,531, U.S. Pat. No. 4,179,337, U.S. Pat. No. 5,122,614, U.S. Pat. No. 5,349,052, U.S. Pat. No. 5,552,520, U.S. Pat. No. 6,127,355, WO 95/06058, WO 98/32466, WO 03/026591, WO 03/057235, WO 03/027637, and WO 2004/066966, which are incorporated herein by reference; (51) Neuropeptide Y2 (NPY2) receptor agonists such NPY3-36, N acetyl [Leu(28,31)] NPY 24-36, TASP-V, and cyclo-(28/32)-Ac-[Lys28-Glu32]-(25-36)-pNPY; (52) Neuropeptide Y4 (NPY4) agonists such as pancreatic peptide (PP) as described in Batterham et al., J. Clin. Endocrinol. Metab. 88:3989-3992 (2003), and other Y4 agonists such as 1229U91; (54) cyclo-oxygenase-2 inhibitors such as etoricoxib, celecoxib, valdecoxib, parecoxib, lumiracoxib, BMS347070, tiracoxib or JTE522, ABT963, CS502 and GW406381, and pharmaceutically acceptable salts thereof; (55) Neuropeptide Y1 (NPY1) antagonists such as BIBP3226, J-115814, BIBO 3304, LY-357897, CP-671906, GI-264879A and those disclosed in U.S. Pat. No. 6,001,836; and PCT Application Nos. WO 96/14307, WO 01/23387, WO 99/51600, WO 01/85690, WO 01/85098, WO 01/85173, and WO 01/89528; (56) Opioid antagonists such as nalmefene (Revex®), 3-methoxynaltrexone, naloxone, naltrexone, and those disclosed in: PCT Application No. WO 00/21509; (57) 11β HSD-1 (11-beta hydroxy steroid dehydrogenase type 1) inhibitor such as BVT 3498, BVT 2733, and those disclosed in WO 01/90091, WO 01/90090, WO 01/90092, and U.S. Pat. No. 6,730,690 and US Publication No. US 2004-0133011, which are incorporated by reference herein in their entirety; and (58) aminorex; (59) amphechloral; (60) amphetamine; (61) benzphetamine; (62) chlorphentermine; (63) clobenzorex; (64) cloforex; (65) clominorex; (66) clortermine; (67) cyclexedrine; (68) dextroamphetamine; (69) diphemethoxidine, (70) N-ethylamphetamine; (71) fenbutrazate; (72) fenisorex; (73) fenproporex; (74) fludorex; (75) fluminorex; (76) furfurylmethylamphetamine; (77) levamfetamine; (78) levophacetoperane; (79) mefenorex; (80) metamfepramone; (81) methamphetamine; (82) norpseudoephedrine; (83) pentorex; (84) phendimetrazine; (85) phenmetrazine; (86) picilorex; (87) phytopharm 57; (88) zonisamide, and (89) Neurokinin-1 receptor antagonists (NK-1 antagonists) such as the compounds disclosed in: U.S. Pat. Nos. 5,162,339, 5,232,929, 5,242,930, 5,373,003, 5,387,595, 5,459,270, 5,494,926, 5,496,833, and 5,637,699; PCT International Patent Publication Nos. WO 90/05525, 90/05729, 91/09844, 91/18899, 92/01688, 92/06079, 92/12151, 92/15585, 92/17449, 92/20661, 92/20676, 92/21677, 92/22569, 93/00330, 93/00331, 93/01159, 93/01165, 93/01169, 93/01170, 93/06099, 93/09116, 93/10073, 93/14084, 93/14113, 93/18023, 93/19064, 93/21155, 93/21181, 93/23380, 93/24465, 94/00440, 94/01402, 94/02461, 94/02595, 94/03429, 94/03445, 94/04494, 94/04496, 94/05625, 94/07843, 94/08997, 94/10165, 94/10167, 94/10168, 94/10170, 94/11368, 94/13639, 94/13663, 94/14767, 94/15903, 94/19320, 94/19323, 94/20500, 94/26735, 94/26740, 94/29309, 95/02595, 95/04040, 95/04042, 95/06645, 95/07886, 95/07908, 95/08549, 95/11880, 95/14017, 95/15311, 95/16679, 95/17382, 95/18124, 95/18129, 95/19344, 95/20575, 95/21819, 95/22525, 95/23798, 95/26338, 95/28418, 95/30674, 95/30687, 95/33744, 96/05181, 96/05193, 96/05203, 96/06094, 96/07649, 96/10562, 96/16939, 96/18643, 96/20197, 96/21661, 96/29304, 96/29317, 96/29326, 96/29328, 96/31214, 96/32385, 96/37489, 97/01553, 97/01554, 97/03066, 97/08144, 97/14671, 97/17362, 97/18206, 97/19084, 97/19942, 97/21702, and 97/49710.

Examples of other anti-obesity agents that can be employed in combination with a compound of Formula I are disclosed in “Patent focus on new anti-obesity agents,” Exp. Opin. Ther. Patents, 10: 819-831 (2000); “Novel anti-obesity drugs,” Exp. Opin. Invest. Drugs, 9: 1317-1326 (2000); and “Recent advances in feeding suppressing agents: potential therapeutic strategy for the treatment of obesity, Exp. Opin. Ther. Patents, 11: 1677-1692 (2001). The role of neuropeptide Y in obesity is discussed in Exp. Opin. Invest. Drugs, 9: 1327-1346 (2000). Cannabinoid receptor ligands are discussed in Exp. Opin. Invest. Drugs, 9: 1553-1571 (2000).

Examples of other active ingredients that may be combined with a compound of Formula I for the treatment or prevention of male or female sexual dysfunction, in particular, male erectile dysfunction, either administered separately or in the same pharmaceutical compositions, include, but are not limited to (a) type V cyclic-GMP-specific phosphodiesterase (PDE-V) inhibitors, including sildenafil and (6R, 12aR)-2,3,6,7,12,12a-hexahydro-2-methyl-6-(3,4-methylenedioxyphenyl)-pyrazino[2′,′:6,1]pyrido[3,4-b]indole-1,4-dione (IC-351); (b) alpha-adrenergic receptor antagonists, including phentolamine and yohimbine or pharmaceutically acceptable salts thereof; (c) dopamine receptor agonists, such as apomorphine or pharmaceutically acceptable salts thereof; and (d) nitric oxide (NO) donors.

The instant invention also includes administration of a single pharmaceutical dosage formulation which contains both the MC-4R agonist in combination with a second active ingredient, as well as administration of each active agent in its own separate pharmaceutical dosage formulation. Where separate dosage formulations are used, the individual components of the composition can be administered at essentially the same time, i.e., concurrently, or at separately staggered times, i.e. sequentially prior to or subsequent to the administration of the other component of the composition. The instant invention is therefore to be understood to include all such regimes of simultaneous or alternating treatment, and the terms “administration” and “administering” are to be interpreted accordingly. Administration in these various ways are suitable for the present compositions as long as the beneficial pharmaceutical effect of the combination of the MC-4R agonist and the second active ingredient is realized by the patient at substantially the same time. Such beneficial effect is preferably achieved when the target blood level concentrations of each active ingredient are maintained at substantially the same time. It is preferred that the combination of the MC-4R agonist and the second active ingredient be co-administered concurrently on a once-a-day dosing schedule; however, varying dosing schedules, such as the MC-4R agonist once a day and the second active ingredient once, twice or more times per day or the MC-4R agonist three times a day and the second active ingredient once, twice or more times per day, is also encompassed herein. A single oral dosage formulation comprised of both a MC-4R agonist and a second active ingredient is preferred. A single dosage formulation will provide convenience for the patient, which is an important consideration especially for patients with diabetes or obese patients who may be in need of multiple medications.

The compounds in the combinations of the present invention may be administered separately, therefore the invention also relates to combining separate pharmaceutical compositions into a kit form. The kit, according to this invention, comprises two separate pharmaceutical compositions: a first unit dosage form comprising a prophylactically or therapeutically effective amount of the melanocortin-4 receptor agonist, or a pharmaceutically acceptable salt or ester thereof, and a pharmaceutically acceptable carrier or diluent in a first unit dosage form, and a second unit dosage form comprising a prophylactically or therapeutically effective amount of the second active ingredient or drug, or a pharmaceutically acceptable salt or ester thereof, and a pharmaceutically acceptable carrier or diluent in a second unit dosage form. In one embodiment, the kit further comprises a container. Such kits are especially suited for the delivery of solid oral forms such as tablets or capsules. Such a kit preferably includes a number of unit dosages. Such kits can include a card having the dosages oriented in the order of their intended use. An example of such a kit is a “blister pack”. Blister packs are well known in the packaging industry and are widely used for packaging pharmaceutical unit dosage forms. If desired, a memory aid can be provided, for example in the form of numbers, letters, or other markings or with a calendar insert, designating the days or time in the treatment schedule in which the dosages can be administered.

Another aspect of the present invention provides pharmaceutical compositions which comprise a compound of Formula I, as an active ingredient or a pharmaceutically acceptable salt thereof, and may also contain a pharmaceutically acceptable carrier and optionally other therapeutic ingredients. The term “pharmaceutically acceptable salts” refers to salts prepared from pharmaceutically acceptable non-toxic bases or acids including inorganic bases or acids and organic bases or acids.

The compositions include compositions suitable for oral, rectal, topical, parenteral (including subcutaneous, intramuscular, and intravenous), ocular (ophthalmic), pulmonary (nasal or buccal inhalation), or nasal administration, although the most suitable route in any given case will depend on the nature and severity of the conditions being treated and on the nature of the active ingredient. They may be conveniently presented in unit dosage form and prepared by any of the methods well-known in the art of pharmacy.

In practical use, the compounds of Formula I can be combined as the active ingredient in intimate admixture with a pharmaceutical carrier according to conventional pharmaceutical compounding techniques. The carrier may take a wide variety of forms depending on the form of preparation desired for administration, e.g., oral or parenteral (including intravenous). In preparing the compositions for oral dosage form, any of the usual pharmaceutical media may be employed, such as, for example, water, glycols, oils, alcohols, flavoring agents, preservatives, coloring agents and the like in the case of oral liquid preparations, such as, for example, suspensions, elixirs and solutions; or carriers such as starches, sugars, microcrystalline cellulose, diluents, granulating agents, lubricants, binders, disintegrating agents and the like in the case of oral solid preparations such as, for example, powders, hard and soft capsules and tablets, with the solid oral preparations being preferred over the liquid preparations.

Because of their ease of administration, tablets and capsules represent the typical oral dosage unit form, in which case solid pharmaceutical carriers are typically employed. If desired, tablets may be coated by standard aqueous or nonaqueous techniques. Such compositions and preparations should contain at least 0.1 percent of active compound. The percentage of active compound in these compositions may, of course, be varied and may conveniently be between about 2 percent to about 60 percent of the weight of the unit. The amount of active compound in such therapeutically useful compositions is such that an effective dosage will be obtained. The active compounds can also be administered intranasally as, for example, liquid drops or spray.

The tablets, pills, capsules, and the like may also contain a binder such as gum tragacanth, acacia, corn starch or gelatin; excipients such as dicalcium phosphate; a disintegrating agent such as corn starch, potato starch, alginic acid; a lubricant such as magnesium stearate; and a sweetening agent such as sucrose, lactose or saccharin. When a dosage unit form is a capsule, it may contain, in addition to materials of the above type, a liquid carrier such as a fatty oil. Various other materials may be present as coatings or to modify the physical form of the dosage unit. For instance, tablets may be coated with shellac, sugar or both. A syrup or elixir may contain, in addition to the active ingredient, sucrose as a sweetening agent, methyl and propylparabens as preservatives, a dye and a flavoring such as cherry or orange flavor.

Compounds of formula I may also be administered parenterally. Solutions or suspensions of these active compounds can be prepared in water suitably mixed with a surfactant such as hydroxy-propylcellulose. Dispersions can also be prepared in glycerol, liquid polyethylene glycols and mixtures thereof in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (e.g. glycerol, propylene glycol and liquid polyethylene glycol), suitable mixtures thereof, and vegetable oils.

The compounds of structural formula I of the present invention can be prepared according to the procedures of the following Schemes and Examples, using appropriate materials and are further exemplified by the following specific examples. The Intermediates and Examples are provided to illustrate the invention and are not to be construed as limiting the scope of the invention in any manner. By utilizing the procedures disclosed herein, one of ordinary skill in the art can readily prepare additional compounds of the present invention claimed herein. The compounds illustrated in the examples are not, however, to be construed as forming the only genus that is considered as the invention. The Examples further illustrate details for the preparation of the compounds of the present invention. Those skilled in the art will readily understand that known variations of the conditions and processes of the following preparative procedures can be used to prepare these compounds. The instant compounds are generally isolated in the form of their pharmaceutically acceptable salts, such as those described previously hereinabove. The free amine bases corresponding to the isolated salts can be generated by neutralization with a suitable base, such as aqueous sodium hydrogencarbonate, sodium carbonate, sodium hydroxide, and potassium hydroxide, and extraction of the liberated amine free base into an organic solvent followed by evaporation. The amine free base isolated in this manner can be further converted into another pharmaceutically acceptable salt by dissolution in an organic solvent followed by addition of the appropriate acid and subsequent evaporation, precipitation, or crystallization. All temperatures are degrees Celsius unless otherwise noted. Mass spectra (MS) were measured by electron-spray ion-mass spectroscopy.

The phrase “standard peptide coupling reaction conditions” means coupling a carboxylic acid with an amine using an acid activating agent such as EDC, DCC, and BOP in an inert solvent such as dichloromethane in the presence of a catalyst such as HOBT. The use of protecting groups for the amine and carboxylic acid functionalities to facilitate the desired reaction and minimize undesired reactions is well documented. Conditions required to remove protecting groups are found in standard textbooks such as Greene, T, and Wuts, P. G. M., Protective Groups in Organic Synthesis, John Wiley & Sons, Inc., New York, N.Y., 1991. CBZ and BOC are commonly used protecting groups in organic synthesis, and their removal conditions are known to those skilled in the art. For example, CBZ may be removed by catalytic hydrogenation in the presence of a noble metal or its oxide such as palladium on activated carbon in a protic solvent such as methanol or ethanol. In cases where catalytic hydrogenation is contraindicated due to the presence of other potentially reactive functionalities, removal of CBZ groups can also be achieved by treatment with a solution of hydrogen bromide in acetic acid or by treatment with a mixture of TFA and dimethylsulfide. Removal of BOC protecting groups is carried out with a strong acid, such as trifluoroacetic acid, hydrochloric acid, or hydrogen chloride gas, in a solvent such as methylene chloride, methanol, or ethyl acetate.

Abbreviations Used in the Description of the Preparation of the Compounds of the Present Invention: Ac is acetate, BOC (boc) is t-butyloxycarbonyl, BOP is benzotriazol-1-yloxytris(dimethyl-amino)phosphonium hexafluorophosphate, Bu is butyl, calc. is calculated, CBZ (Cbz) is benzyloxycarbonyl, DEAD is diethyl azodicarboxylate, DIEA or DIPEA is N,N-diisopropylethylamine, DMAP is 4-dimethylaminopyridine, DMF is N,N-dimethylformamide, DMSO is dimethylsulfoxide, EDC is 1-(3-dimethyl-aminopropyl)-3-ethylcarbodiimide HCl, eq. is equivalent(s), ES-MS and ESI-MS are electron spray ion-mass spectroscopy, Et is ethyl, EtOAc is ethyl acetate, HATU is O-(7-azabenzotriazol-1-yl)-1,1,3,3-tetramethyluronium hexafluoro-phosphate, HOAt is 1-hydroxy-7-azabenzotriazole, HOBt is 1-hydroxybenzotriazole hydrate, HPLC is high performance liquid chromatography, hr(s) is hour(s); IPA is isopropyl alcohol; LiHMDS is lithium hexamethyl disilazane, LDA is lithium diisopropylamide, Me is methyl, MeOH is methanol, MF is molecular formula, mmol is millimole(s), MPLC is medium pressure liquid chromatography, MS is mass spectrum, Ms is methanesulfonyl, MTBE is tert-butyl methyl ether, NMM is N-methylmorpholine, OTf is trifluoromethane-sulfonyl, Ph is phenyl, Pr is propyl, prep. is prepared, PyBOP is benzotriazol-1-yloxytripyrrolidine phosphonium hexafluorophosphate, r.t. is room temperature, (S)-2-methyl-CBS-oxazaborolidine is (S)-tetrahydro-1-methyl-3,3-diphenyl-1H,3H-pyrrolo[1,2-c][1,3,2]oxazaborole, TEA is triethylamine, TFA is trifluoroacetic acid, THF is tetrahydrofuran, and TLC or tic is thin-layer chromatography.

Reaction Schemes A-G illustrate methods employed in the synthesis of the compounds of the present invention of structural formula I. All substituents are as defined above unless indicated otherwise.

Reaction Scheme A illustrates a key step in the synthesis of the novel compounds of structural formula I. As shown in Scheme A, the reaction of a piperidine or piperazine derivative of type 1, wherein Z is N or CR⁴, with a carboxylic acid derivative of formula 2 affords a title compound of structural formula I. The amide bond coupling reaction illustrated in reaction Scheme A is conducted in an appropriate inert solvent such as methylene chloride or the like and may be performed with a variety of reagents suitable for amide coupling reactions such as EDC, HATU, or PyBOP. Preferred conditions for the amide bond coupling reaction shown in reaction Scheme A are known to those skilled in organic synthesis. Modifications of these reaction conditions may include, but are not limited to, the use of basic reagents such as NMM, TEA, or DIPEA, or the addition of an additive such as HOAt or HOBt. Alternatively, 4-substituted piperidines of formula 1 may be treated with an active ester or acid chloride derived from carboxylic acid 2 which also affords compounds of structural formula I. The amide bond coupling shown in reaction Scheme A is usually conducted at a temperature between 0° C. and room temperature, occasionally at elevated temperatures, and the coupling reaction is typically conducted for periods of 1 to 24 hours.

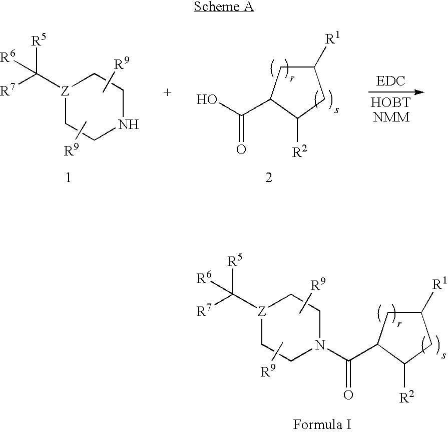

The synthesis of carboxylic acids of general formula 2 utilized in the amide bond coupling reaction in Scheme A was previously described in WO 02/067869 (6 Sep. 2002). Reaction Reaction Schemes B-F illustrate methods for the synthesis of the carboxylic acids of general formula 2 that are utilized in the amide bond coupling reaction shown in reaction Scheme A. Schemes G and H illustrate methods for preparing the piperidine and piperazine compounds 1.

Reaction Schemes B and C illustrate the synthesis of the novel compounds of structural formula I when it is preferred to effect the amide bond coupling step prior to incorporation of the basic substituent R¹ as mentioned above. Reaction Scheme B illustrates a method for the synthesis of compounds of structural formula I which employs a piperidine or piperazine of general formula 1 and a cycloalkanone carboxylic acid of general formula 3 as the partners in the amide bond coupling step. The piperidine/piperazine of formula 1 and the carboxylic acid of formula 3 are first coupled to afford an amide of general formula 4 using the reagents and conditions described for the generalized amide coupling shown in reaction Scheme A. The R¹ substituent (R¹═N containing heterocycloalkyl or NR⁹R⁹) may then be incorporated at the position of the carbonyl group by performing a reductive amination reaction with an amine of general formula 5. Treatment with a secondary or primary amine NR⁹R⁹ gives the tertiary amine substituted cyclopentyl compounds (R¹═NR⁹R⁹) of formula I as shown in Scheme B, whereas treatment with an amine containing heterocycloalkyl, such as piperidine, gives the heterocycloalkyl substituted cyclopentyl compounds (R¹=heterocycloalkyl) of formula I. Typical conditions for effecting such a reductive amination include preforming an imine 6 from ketone 3 and amine 5 followed by reduction of the intermediate imine with reducing agents such as sodium borohydride, sodium cyanoborohydride or sodium triacetoxyborohydride. Formation of the intermediate imine 6 derived from piperidine 1 and acid 3 may occur spontaneously in solution or it may be promoted with agents such as titanium (IV) isopropoxide in a solvent such as methanol or with anhydrous magnesium sulfate in chloroform. The formation of the imine 6 is generally performed at temperatures between 0° C. and the reflux temperature of the solvent being used, frequently at room temperature. The imine formation step is generally allowed to proceed to completion over a period of several hours to 1 day prior to the reduction step which minimizes the formation of secondary alcohols formed by simple reduction of the keto group in compounds of general formula 4. The intermediate imine 6 may in some cases be isolated and purified, however it is generally preferred to use it directly in the reduction step. The reduction of the imine 6 is typically conducted in an alcoholic solvent such as methanol or ethanol at temperatures between 0° C. and room temperature, and the reduction is generally completed in periods of several hours or less.

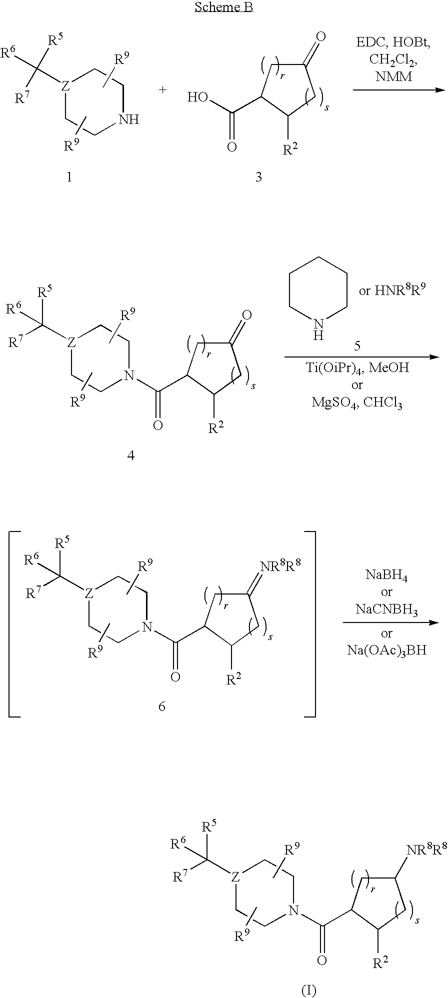

Reaction Scheme C illustrates a preferred method for the synthesis of compounds of structural formula I which employs a piperidine or piperazine of general formula 1 and a hydroxyl-substituted cycloalkyl carboxylic acid of general formula 7 as the partners in the amide bond coupling step. The amide bond coupling step between piperidine 1 and carboxylic acid 7 is performed first, typically using a carbodiimide reagent like EDC to promote the coupling as described above. The hydroxyl-substituted amide 8 which is produced is then further synthetically modified to incorporate the R¹ substituent present in the title compounds of structural formula I. A variety of methods known to those skilled in organic synthesis may be used to incorporate the R¹ substituent. For instance, the hydroxyl group of compounds of general formula 8 may be oxidized using a variety of methods to afford carbonyl compounds of general formula 4. The resulting ketoamides of general formula 4 may then be converted to the title compounds of structural formula I using the reductive amination reaction described in reaction Scheme B.

Occasionally, it may be preferable to utilize hydroxyl-substituted compounds of general formula 8 in a Fukuyama-Mitsunobu reaction (Fukuyama, T.; Cheung, M.; Jow, C.-K.; Hidai, Y.; Kan, T. Tetrahedron Lett. 1997, 33, 5831-4) sequence as shown in reaction Scheme C. In this method for the synthesis of the novel title compounds of structural formula I, the intermediate hydroxyl-substituted cycloalkylamide 8 is reacted with a 2,4-dinitrobenzene-sulfonamide of general formula 9 in the presence of triphenylphosphine and an azodicarboxylate reagent such as diethyl azodicarboxylate (DEAD). The reaction is performed in a suitable aprotic solvent such as benzene, toluene or tetrahydrofuran, typically at room temperature, and the reaction is generally complete in 0.5-3 hours. The product of this reaction is the secondary 2,4-dinitrobenzenesulfonamide of general formula 10, which may then be readily converted to a title compound of structural formula I wherein R⁹═H. The deprotection of the sulfonamide group is accomplished by reaction of 10 with either a base like n-propylamine in a solvent like methylene chloride or by reaction of 10 with a nucleophilic reagent such as mercaptoacetic acid with triethylamine in methylene chloride. In either case the reaction is typically conducted at room temperature, for periods of 5 minutes to one hour. An advantage of the Fukuyama-Mitsunobu reaction sequence is that the stereochemistry of the carbon atom undergoing substitution is cleanly inverted. Thus if the hydroxyl-substituted cycloalkylamide 8 is a single diastereoisomer, then the product 10 will be a single diastereoisomer also. This is in contrast to the reductive amination strategy discussed in reaction Scheme B which generally affords a mixture of epimeric products.

The secondary amine of formula I (R⁹═H) shown in reaction Scheme C may then be further synthetically modified using a variety of methods known in organic synthesis to incorporate other embodiments of the R⁹ substituent. For instance, compounds of structural formula I where R⁹═H may be subjected to a reductive amination reaction with an appropriate aldehyde or ketone using the conditions described in reaction Scheme B.

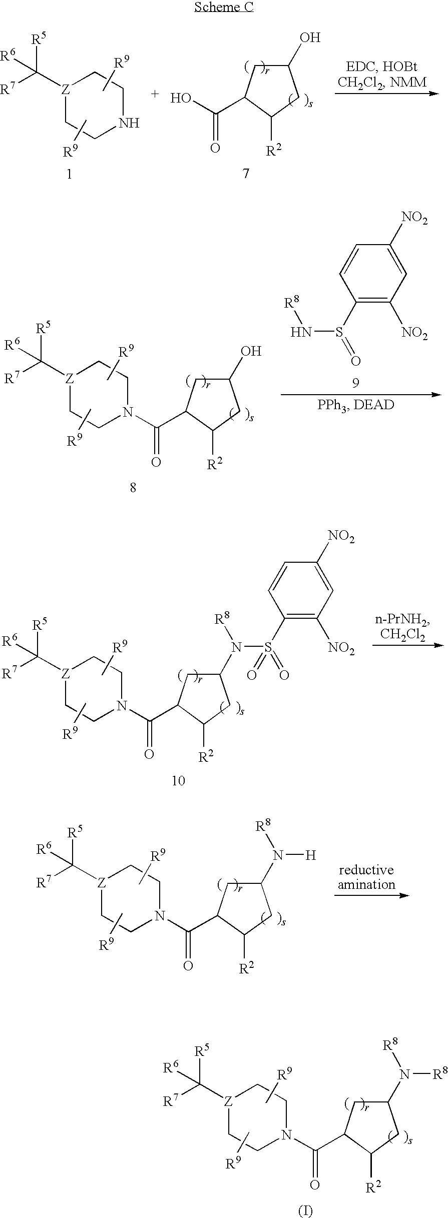

Reaction Scheme D illustrates a preferred method for the synthesis of the cycloalkyl carboxylic acids of general formula 3 when the values of r and s are selected such that the resulting carbocyclic ring is a six-membered ring. In this method a Diels-Alder reaction between an α,β-unsaturated ester of general formula 11 and 2-trimethylsilyloxybutadiene (12) affords a mixture of the two regioisomeric silylenolethers 13 and 14. The silylenolethers 13 and 14 are generally subjected to an hydrolysis reaction using hydrochloric acid in a solvent such as methanol and the two regioisomeric ketones 15 and 16 are then separated by conventional chromatographic methods. The olefin geometry of the starting α,β-unsaturated ester of general formula 11 determines the relative stereochemistry of the two substituents on the six-membered ring. Thus a trans α,β-unsaturated ester (11) affords the trans-disubstituted products 13 and 14 as shown, whereas the corresponding cis isomer of compounds of general formula 11 will afford the corresponding cis isomers of 13 and 14. Once the regioisomeric cyclohexanones of general formulae 15 and 16 are separated, they may then be individually hydrolyzed. For instance, hydrolysis using lithium hydroxide in refluxing tetrahydrofuran, affords the carboxylic acids of general formula 3 (r=2, s=1) and 3 (r=1, s=2). The acids of general formula 3 are finally converted to the novel title compounds of structural formula I using the methodology described above in reaction Scheme B.

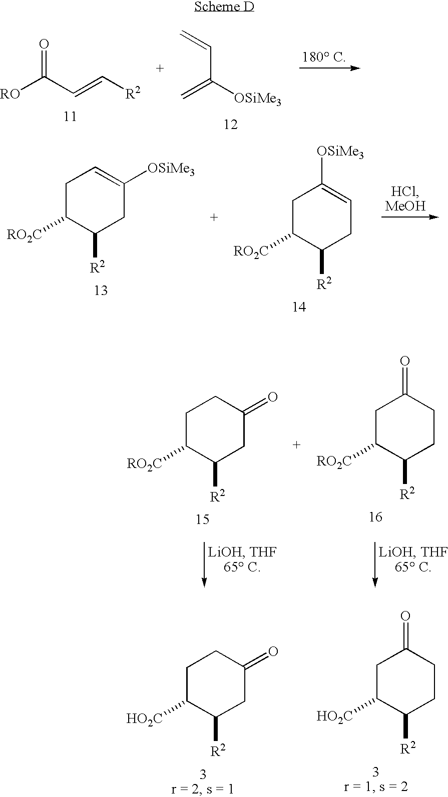

Reaction Scheme E illustrates a preferred method for the synthesis of the cycloalkyl carboxylic acids of general formula 3 when the values of r and s are selected such that the resulting carbocyclic ring is a five-membered ring. In this method an α,β-unsaturated ester of general formula 11 is subjected to a trimethylenemethane cycloaddition reaction (Trost, B. M.; Chan, D. M. T. J. Am. Chem. Soc. 1979, 101, 6429) to afford a cyclopentane derivative of general formula 18. The cycloaddition is performed by reacting the α,β-unsaturated ester of general formula 11 with 2-[(trimethylsilyl)methyl]-2-propen-1-yl acetate (17) in the presence of a palladium(0) catalyst in a solvent such as tetrahydrofuran. A preferred palladium(0) catalyst for the cycloaddition may be generated by mixing palladium acetate and triisopropyl phosphite in the reaction mixture. The cycloaddition reaction is typically conducted at the reflux temperature of the solvent, for instance 65° C., and the reaction is usually completed in periods of 2-8 hours. The olefin geometry of the starting α,β-unsaturated ester of general formula 11 determines the relative stereochemistry of the two substituents on the five-membered ring. Thus a trans α,β-unsaturated ester (11) affords the trans-disubstituted product 18 as shown, whereas the corresponding cis isomer of compounds of general formula 11 affords the corresponding cis-disubstituted isomer of 18. The exocyclic olefin present in compounds of general formula 18 is next oxidatively removed to afford a cyclopentanone derivative of general formula 19. A preferred method for the oxidative cleavage reaction is the two step process shown at the bottom of reaction Scheme E. The methylene cyclopentane derivative of formula 18 is first oxidized to a 1,2-diol derivative using catalytic osmium tetraoxide in the presence of a stoichiometric reoxidant such as N-methylmorpholine-N-oxide and a solvent system such as acetone-water. The intermediate 1,2-diol which forms is generally not isolated, but is in turn subjected to cleavage with sodium periodate in a solvent system like methanol-water to afford ketones of general formula 19. Both steps in the oxidative cleavage sequence are generally completed during periods of several minutes to a few hours and the reaction steps are typically conducted at low temperatures, for instance between 0° C. and room temperature. Alternatively, the oxidative cleavage of olefins of general formula 18 may be accomplished using ozone, or by other methods known in organic synthesis. The cyclopentanones of general formula 19 may then be hydrolyzed, for instance using sodium hydroxide in methanol, to afford the carboxylic acids of general formula 3 (r=1, s=1). The acids of general formula 3 are finally converted to the novel title compounds of structural formula I using the methodology described above in reaction Scheme B.

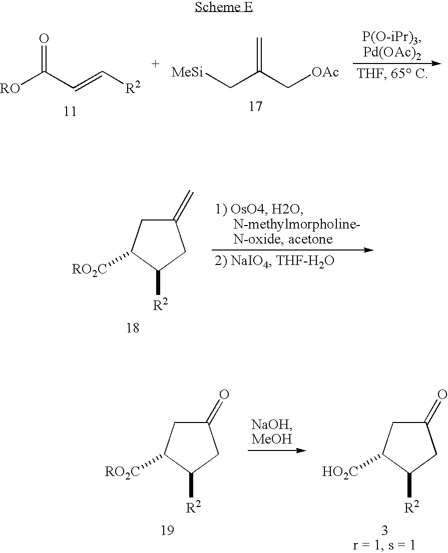

When it is desired to prepare individual enantiomers of the novel title compounds of structural formula I, it is possible to perform a resolution of the compounds of structural formula I using one of the methods known in the art of organic synthesis. For instance, enantiomerically pure compounds (I) may be prepared by crystallization of diastereoisomeric salts formed from the racemic compounds of structural formula I and an optically active carboxylic acid. The two diastereoisomeric salts are separated from each other by fractional crystallization, then the enantiomerically pure compounds of structural formula I are regenerated by treatment of the purified salts with a base. Alternatively, racemic compounds of structural formula I may be resolved by preparative HPLC using commercially available chiral-stationary phase columns. Another strategy for the preparation of enantiomerically pure compounds of structural formula I involves preparing enantiomerically pure compounds of general formula 2 prior to their use in the amide bond forming reaction outlined in reaction Scheme A. Racemic compounds of general formula 2, or intermediates used to prepare compounds of formula 2 as described in the previous reaction Schemes (i.e. acids 3 and 7, or esters 15, 16 and 19) may also be resolved using the classical methods previously discussed.

Enantiomerically pure compounds may also be prepared from starting materials bearing a suitable covalently attached chiral auxiliary group using synthetic transformations similar to those outlined above. Reaction Scheme F illustrates the use of a covalently attached chiral oxazolidinone auxiliary for the preparation of enantiomerically pure cyclopentanones of general formula 19. In this method of preparation, an α,β-unsaturated acyloxazolidone of general formula 20 is subjected to the trimethylenemethane cycloaddition reaction with compound 17 as described above in reaction Scheme E. The α,β-unsaturated acyloxazolidones of general formula 20 are readily prepared from α,β-unsaturated carboxylic acids and (S)-(−)-4-benzyl-2-oxazolidinone using published methodology (Ho, G.-J.; Mathre, D. J. J. Org. Chem. 1995, 60, 2271 and references cited therein). The compounds of general formula 20 undergo the trimethylenemethane cycloaddition under the same conditions as compounds of general formula 11 (Scheme E) and the products are the diastereoisomeric cyclopentanes 21 and 22. Compounds of general formulae 21 and 22 are readily separated from each other by chromatographic methods or by recrystallization, and may then be converted to the compounds of general formula 19 individually. This process is illustrated at the bottom of reaction Scheme F for the case of the cyclopentane with the absolute stereochemistry shown in formula 21. The enantiomerically pure compounds of general formula 21 are first hydrolyzed to afford intermediate carboxylic acids and (S)-(−)-4-benzyl-2-oxazolidinone using a reagent such as lithium hydroperoxide in a suitable solvent system such as aqueous tetrahydrofuran. The carboxylic acid formed is generally then converted to a methyl ester 23 using diazomethane, trimethylsilyldiazomethane or any of the esterification methods commonly employed in organic synthesis. The olefin present in the esters of general formula 23 is then subjected to the oxidative cleavage reaction presented in the discussion of reaction Scheme E to afford enantiomerically pure compounds of general formula 19. The compounds of general formula 19 may then be converted into enantiomerically pure compounds of structural formula I as discussed previously.

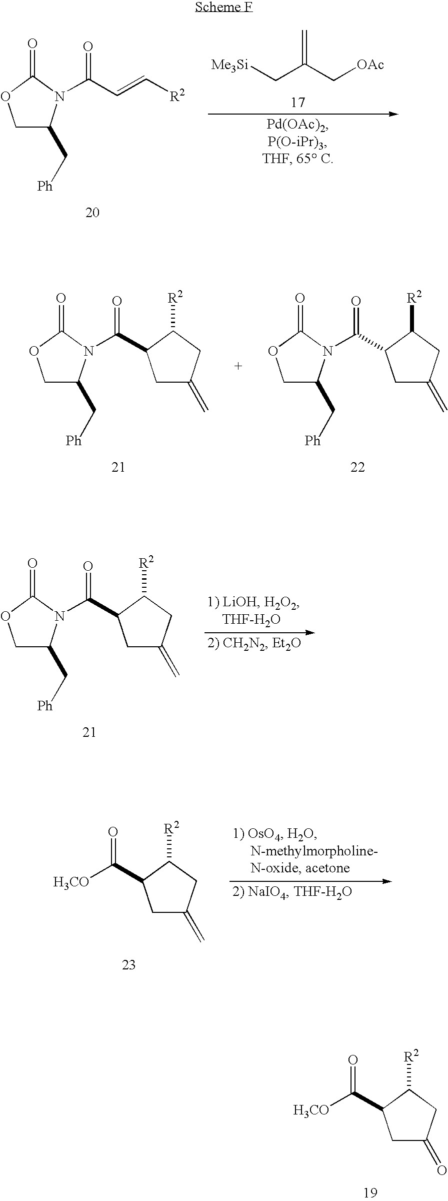

Reaction Schemes G and H illustrate methods for the synthesis of R⁵, R⁶ and R⁷ substituted piperidine sidechains useful to prepare compounds of structural formula I. As shown in Scheme G, 3-chloropyridine 24 is treated with 3,3-dimethylbutanal in a solvent such as THF to give hydroxy alkyl pyridine 25. The hydroxy group of 25 is oxidized to the ketone and subsequently treated with triethyl phosphonoacetate to give alkene acid 27 via a Horner-Emmons reaction. Hydrogenation of the pyridine with Pt₂ catalyst, and treatment with boc anhydride gives the piperidine 28. The ester of 29 is hydrolyzed to the acid, and after a series of protection and deprotection steps, the ester 34 can be further derivatized to give the alcohol 35 via a Grignard reaction with MeMgBr and to give amide 36 by treatment of the alcohol with CH₃CN in the presence of H₂SO₄.

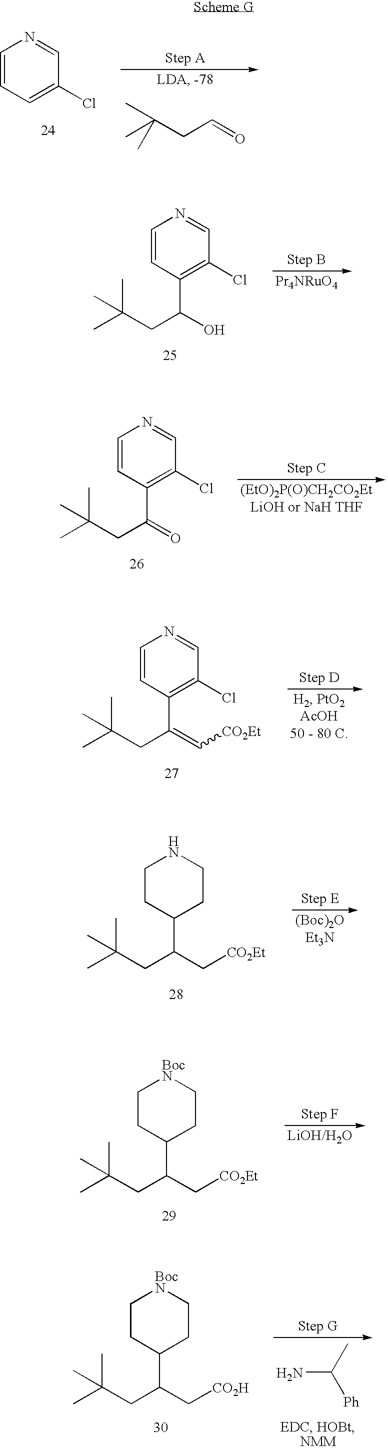

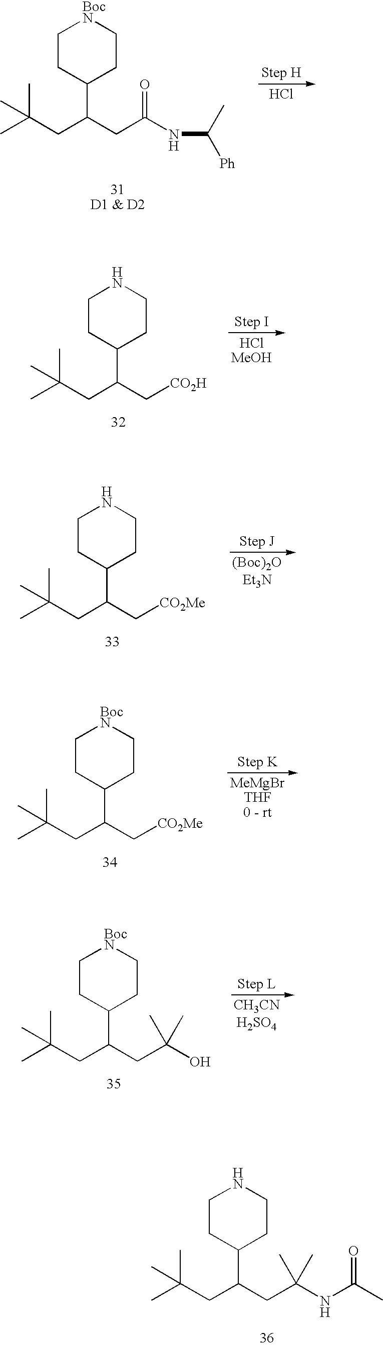

Alternatively, 3-chloropyridine 37 may be derivatized with a cycloalkyl substituent by treatment with N-methoxy-N-methyl-3-cyclopentanecarboxamide 38, which is formed by the treatment of cyclopetane acid with N,O-dimethylhydroxylamine hydrochloride in the presence of HOBT and EDC as shown in Scheme H. The ketone sidechain of pyridine cyclopentyl may be further derivatized as shown Steps C-L of Scheme G.

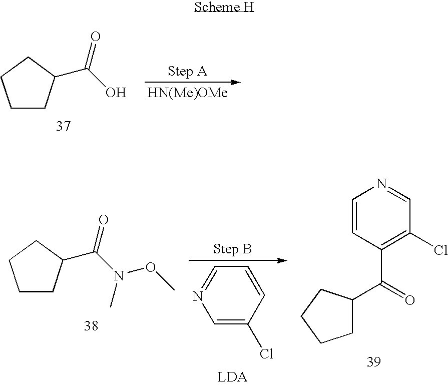

Reaction Scheme I illustrates the general method employed in the synthesis of the piperazine compounds of structural formula I by coupling a 4-substituted piperazine intermediate of general formula I-1 with either a pyrrolidine acid I-2 or piperidine acid I-4 or I-6. The preparation of piperazines of general formula I-1 is provided in general Schemes J-T and in the Examples. The preparation of pyrrolidine acid I-2, piperidine acid I-4, and piperidine acid I-6 is provided in Schemes D-F. All substituents in the Schemes are as defined above unless indicated otherwise.

As illustrated in Scheme I, the amide bond coupling reaction of I-1 to form compounds I-3, I-5, and I-7 is conducted in an appropriate inert solvent such as DMF, methylene chloride or the like and may be performed with a variety of reagents suitable for amide coupling reactions such as HATU, EDC or PyBOP. Preferred conditions for the amide bond coupling reaction shown in reaction Scheme I are known to those skilled in organic synthesis. Such modifications may include, but are not limited to, the use of basic reagents such as TEA or NMM, or the addition of an additive such as HOAt or HOBt. Alternatively, 4-substituted piperazines of formula I-1 may be treated with an active ester or acid chloride derived from carboxylic acid I-2, I-4, or I-6, which also affords compounds of structural formula I-3, I-5, or I-7. The amide bond coupling shown in reaction Scheme I is usually conducted at temperatures between 0° C. and room temperature, occasionally at elevated temperatures, and the coupling reaction is the synthesis and deprotected under acidic conditions, for instance using trifluoroacetic acid in a solvent like methylene chloride or hydrogen chloride in a solvent such as ethyl acetate at room temperature.

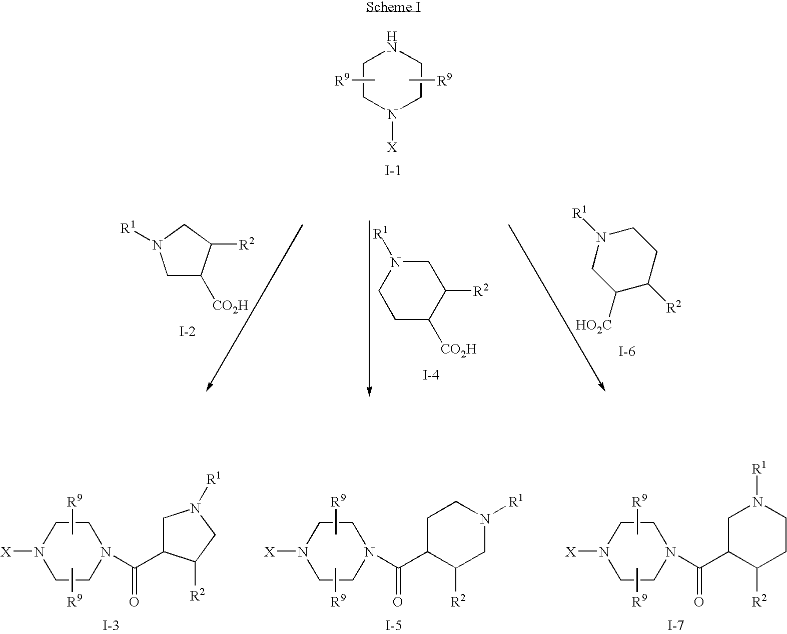

X, R¹, R² and Rare as defined supra.

The synthesis of compounds of general structural formula I-2, I-4 and I-6 is described in WO 02/068387 (6 Sep. 2002); WO 02/068388 (6 Sep. 2002), which are incorporated by reference herein in their entirety.

Scheme J illustrates the preparation of piperazines I-1, wherein X is (CH₂)OC(R⁵)(R⁶)(R⁷), R⁷ is (CH₂)ₙN(R)², n is 1, and R may be as defined in Scheme J. Aldehyde J-1 is converted to the hydroxy nitro compound J-2 by a nitro aldol reaction. Alkene J-3 is formed by subsequent dehydration of J-2. Compound J-5 is formed by the Michael addition of the Boc piperazine J-4 to compound J-3 in an organic solvent, such as methylene chloride at room temperature. The nitro compound J-5 is reduced to form an alkylated amine J-7, followed by removal of the protecting group PG to give piperazine J-8, which corresponds to a compound of general formula I-1.

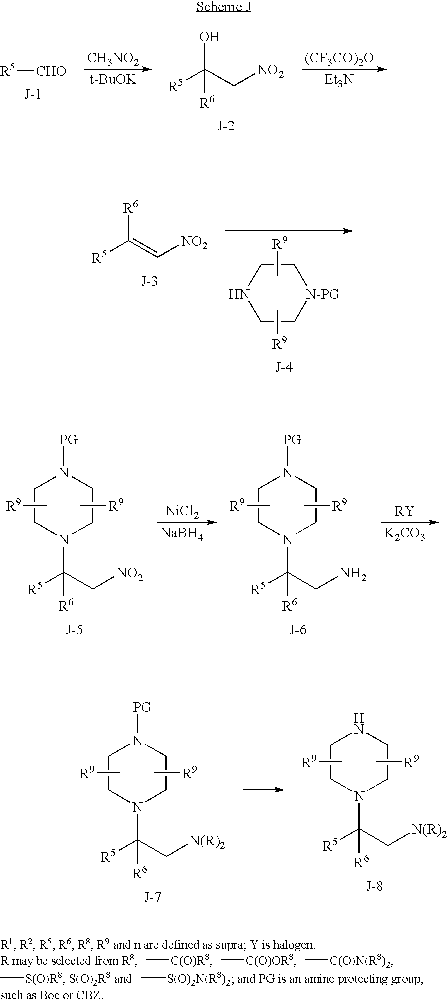

Scheme K illustrates the preparation of piperazines of formula I-1, wherein X is (CH₂)OC(R⁵)(R⁶)(R⁷), R⁷ is (CH₂)ₙN(R)², and n is 3. As shown in Scheme K, ester K-2 may be formed by a Michael addition of nitropiperazine J-5 to methyl acrylate, followed by treatment with tributyl tin hydride and AIBN to remove the nitro group, to yield ester K-3. The ester K-3 may be hydrolyzed to the corresponding acid K-4, which may be subsequently treated with DPPA to form the CBZ protected amine K-5. The CBZ group may be removed under standard conditions known to one skilled in the art to give free amine K-6. The protecting group PG of compound K-6 may be removed to give compound K-7, which corresponds to a compound of formula I-1. Alternatively, compound K-6 may be substituted, as shown in Scheme J, followed by protecting group removal, to give a compound of formula I-1 wherein X is (CH₂)₀C(R⁵)(R⁶)(R⁷), R⁷ is (CH₂)ₙN(R)₂, n is 3 and R may be as defined in Scheme J.

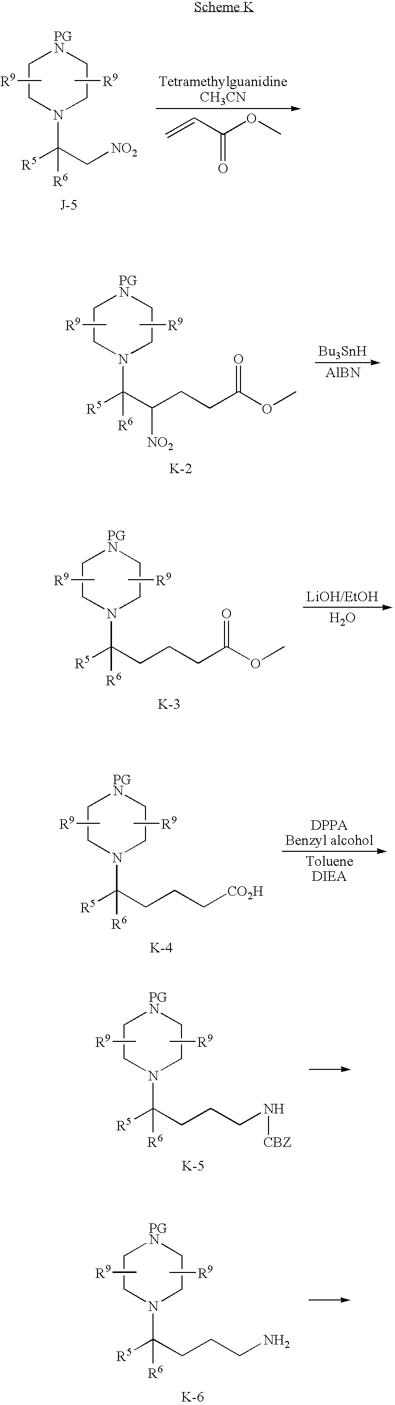

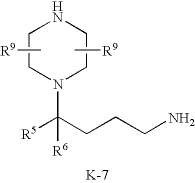

R⁵, R⁶ and R⁶ are as defined supra; PG is a protecting group such as Boc or CBZ.

Reaction Schemes L-Q illustrate preferred methods for the synthesis of alkyl piperidine and piperazine intermediates useful to prepare compounds of structural formula I.

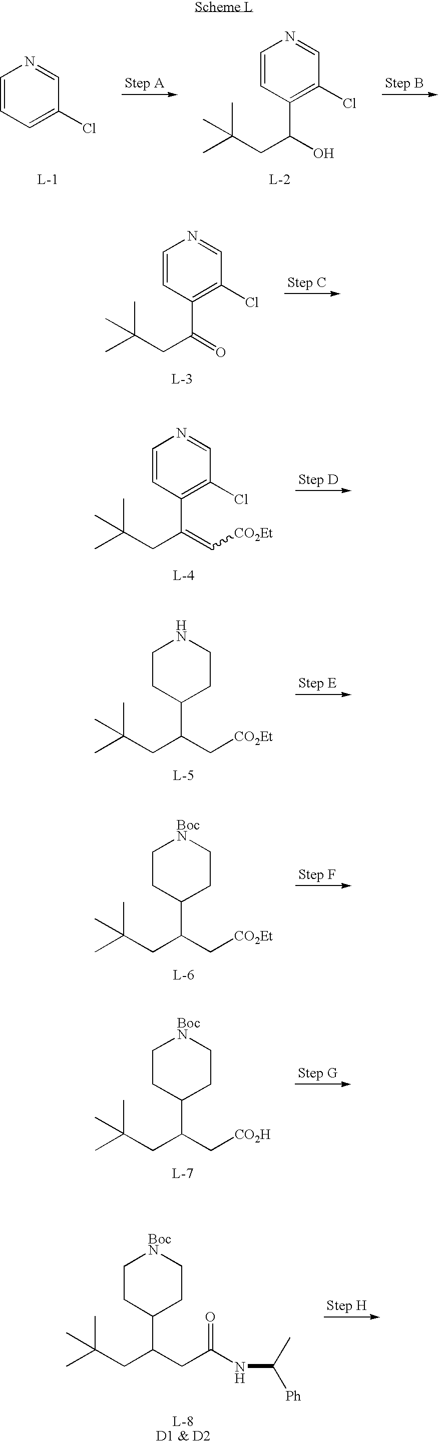

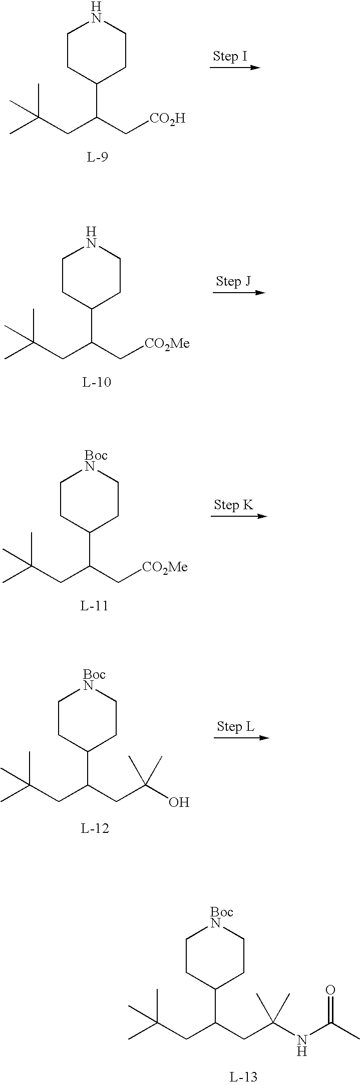

Step A: To a solution of 3-chloropyridine (L-1, 4.54 g, 40 mmol) in THF (40 mL) at −78° was added slowly a solution of LDA (2M, 20 mL, 40 mmol) in 15 minutes. After stirring the reaction mixture for 20 minutes at −78°, a solution of 3,3-dimethylbutanal (4.0 g, 40 mmol) in THF (5 mL) was added dropwise over ˜10 minutes. The reaction mixture was stirred further at −78° for 1 hr, warmed to room temperature, and quenched with aqueous NaHCO₃. The mixture was extracted with ethyl acetate, washed with brine, dried and concentrated to give L-2, which was used without further purification in the next step. ES-MS: Calcd. For C₁₁H₁₄ClNO: 213. Found 214 (M⁺+1).
Step B: To a solution of L-2 (8.45 g, 39.6 mmol) in methylene chloride (50 mL) was added 4 Å molecular sieves (4 g), 4-methylmorpholine N-oxide (6.96 g, 59.5 mmol) and tetrapropylammonium perruthenate (694 mg, 1.98 mmol). After stirring the reaction mixture overnight at room temperature, the mixture was diluted with hexane and filtered through a silica gel plug. The silica gel plug was washed with 3:1 hexane/methylene chloride and combined extract was concentrated to give ketone L-3, which was used as such for further reaction. ES-MS: Calcd. For C₁₁H₁₄ClNO: 211. Found 212 (M⁺+1).
Step C: To a solution of L-3 in THF (100 mL) was added 4 Å molecular sieves (5 g), LiOH.H₂O (3.35 g, 80 mmol) and triethyl phosphonoacetate (17.93 g, 80 mmol). After stirring the reaction mixture at room temperature for 2 days, mixture was filtered and the residue washed with ethyl acetate. The combined organic extracts were washed with brine, dried, concentrated and purified by chromatography over silica gel using 10% ethyl acetate in hexane to give L-4. ES-MS: Calcd. For C₁₅H₂₀ClNO₂: 281. Found 282 (M⁺+1).
Step D: To a solution of L-4 in acetic acid (50 mL) was added platinum oxide (750 mg) and the mixture was stirred at 80° under hydrogen atmosphere overnight. The reaction vessel was flushed with nitrogen, and the mixture filtered and concentrated to give L-5. ES-MS: Calcd. For C₁₅H₂₉NO₂: 255. Found 256 (M⁺+1).
Step E: To a solution of L-5 (7.1 g, 22.53 mmol) in methylene chloride (75 mL) was added triethyl amine (6.8 g, 67.59 mmol) and di t-butyl dicarbonate (4.91 mmol). After stirring the reaction mixture for 4 hr at room temperature, mixture was diluted with methylene chloride, washed with water, dried and concentrated to give L-6. ES-MS: Calcd. For C₂₀H₃₇NO₄: 355. Found 356 (M⁺+1).
Step F: To a solution of L-6 (7.5 g, 21.26 mmol) in ethanol (50 mL) was added a solution of LiOH.H₂O (3.54 g, 84.5 mmol) in water (30 mL). After stirring the reaction mixture at room temperature overnight, the mixture was concentrated, acidified and partitioned between ethyl acetate and water. The organic layer was dried and concentrated to give L-7. ES-MS: Calcd. For C₁₈H₃₃NO₄: 327. Found 328 (M⁺+1).
Step G: To a solution of L-7 (4.7 g, 14.37 mmol) in methylene chloride (40 mL) was added EDC (4.82 g, 25.15 mmol), HOBT (3.39 g, 25.15 mmol), NMM (4.37 g, 43.11 mmol) and (1S)-phenylethylamine (1.74 g, 14.37 mmol). After stirring the reaction mixture at room temperature overnight, the mixture was diluted with methylene chloride, washed with water, aqueous HCl, dried and concentrated. The resulting residue was chromatographed over silica gel using 4% t-butyl methyl ether in methylene chloride to give L-8 D1 and F-8 D2. ES-MS: Calcd. For C₂6H₄₂N₂O₃: 430. Found 431 (M⁺+1).
Step H: A solution of L-8 (D1, 1.73 g, 4.02 mmol) in concentrated HCl (15 mL) was heated in a sealed tube at 130° overnight. The reaction mixture was cooled and concentrated to give L-9 as a white solid, which was used in the next step without further purification. ES-MS: Calcd. For C₁₃H₂₅NO₂: 227. Found 228 (M⁺+1).
Step I: To a solution of L-9 (913 mg, 4.02 mmol) in methanol (30 mL) was added 5 mL of 4N HCl in dioxane. After stirring the reaction mixture for overnight at room temperature, mixture was concentrated to give L-10. ES-MS: Calcd. For C₁₄H₂₇NO₂: 241. Found 242 (M⁺+1).
Step J: To a solution of F-10 (1.1 g, 4.03 mmol) in methylene chloride was added triethylamine (1.938 g, 1916 mmol) and di tert-butyl dicarbonate (1.046 g, 4.8 mmol). After stirring the reaction mixture for over night, reaction was diluted with methylene chloride, washed with water, dried and concentrated to give L-11. ES-MS: Calcd. For C₁₉H₃₅NO₄: 341. Found 342 (M⁺+1).
Step K: To a solution of L-11 (1.36 g, 4.0 mmol) in THF (15 mL) at 0° C. was added dropwise MeMgBr (3M, 4.66 mL, 14 mmol) over ˜10 minutes. The reaction mixture was warmed to room temperature and stirred overnight. The reaction was quenched with aqueous NaHCO₃, extracted with ethyl acetate, dried and concentrated to give L-12, which was used in the next step without further purification. ES-MS: Calcd. For C₂₀H₃₉NO₃: 341. Found 364 (M⁺+23).
Step L: To a solution of L-12 (1.35 g, 3.95 mmol) in CH₃CN (15 mL) at 0° C. was added concentrated H₂SO₄ (1.759 mL, 31.65 mmol). The reaction mixture was warmed to room temperature and stirred for 2 days. Then the mixture was basified with 5N NaOH (15 mL), concentrated and extracted with ethyl acetate. The organic layer was dried and concentrated to give crude L-13. ES-MS: Calcd. For C₁₇H₃₄N₂O: 282. Found 283 (M⁺+1).

Following the synthetic route described in Scheme L and using the appropriate reagents, the following intermediates were prepared:

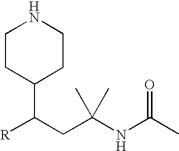

| Compound | | |
|---|---|---|
| No. | R | M⁺ + H |
| L-14 | Isobutyl | 269 |
| L-15 | Isopropyl | 255 |
| L-16 | 3-pentyl | 283 |
| L-17 | CF₃ | 281 |
| L-18 | Cyclobutyl | 267 |
| L-19 | Cyclopentyl | 295 |
| L-20 | Cyclohexyl | 295 |

Scheme M illustrates the preparation of the cyclohexyl BOC piperazine amine intermediate M-6. Other piperazine intermediates may be prepared as shown in Scheme M by substituting the appropriate alkyl, cycloalkyl, heterocycloalkyl, aryl and heteroaryl group for the cyclohexyl group of cyclohexanecarboxaldehyde M-1.

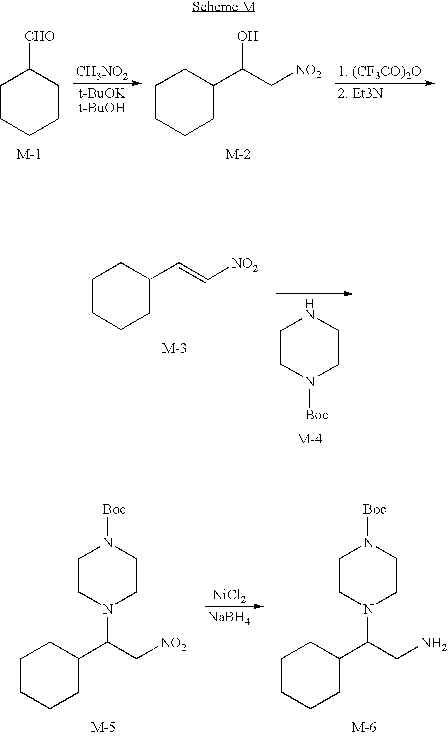

Step A: To a solution of cyclohexanecarboxaldehyde M-1 (4.5 g, 40.1 mmol, Aldrich) in tetrahydrofuran (10.5 mL) and tert-butanol (10.5 mL) was added nitromethane (3.3 mL, 60.2 mmol), followed by addition of potassium tert-butoxide at 0° C. The reaction mixture was stirred at 0° C. for 2 hr, then allowed to warm up to room temperature and stirred overnight. The reaction mixture was poured into water (150 mL) and extracted with t-butyl methyl ether (3×150 mL). The combined organic extracts were washed with brine (100 mL), dried over MgSO₄ and concentrated to give compound M-2. ESI-MS calc. for C₈H₁₅NO₃: 173. Found: 196 (M+Na).
Step B: To a solution of compound M-2 (6.8 g, 39.3 mmol) in dichloromethane (50 mL) was added trifluoroacetic anhydride (5.8 mL, 41.2 mmol) at −10° C. The resulting solution was stirred for 2 minutes, then triethylamine (11.5 mL, 82.5 mmol) was added slowly over 15 minutes. The mixture was stirred for 30 minutes at −10° C., then poured into CH₂Cl₂ (250 mL) and washed with saturated NH₄Cl (2×100 mL). The aqueous layers were back extracted with CH₂Cl₂ (100 mL). The combined organic layers were washed with brine (100 mL), dried over MgSO₄ and concentrated to give a yellow oil, which was purified by chromatography over silica gel (hexane:ethyl acetate=20:1) to give compound M-3. ESI-MS calc. for C₈H₁₃NO₂: 155 Found: 156 (M+H).
Step C: To a solution of compound M-3 (2.41 g, 15.6 mmol) in CH₂Cl₂ (20 mL) was added Boc-piperazine M-4 (2.63 g, 14.1 mmol, Aldrich). The mixture was stirred at room temperature overnight, then concentrated to give a crude product. The crude product was purified by chromatography over silica gel (hexane:ethyl acetate=10:1) to give compound M-5. ESI-MS calc. for C₁₇H₃₁N₃O₄: 341. Found: 342 (M+H).
Step D: To a solution of compound M-5 (1.1 g, 3.2 mmol) in methanol (50 mL) was added nickel (II) chloride hexahydrate (1.92 g, 8.1 mmol). The resulting solution was cooled to 0° C., and sodium borohydride (3.1 g, 80.6 mmol) was added slowly. The mixture was stirred at 0° C. for 2 hr, then concentrated to give a residue. To the residue was added CH₂Cl₂ (250 mL) and NaOH (1N, 250 mL), and the resulting emulsion was filtered though celite. The organic and aqueous layers were separated; the aqueous was extracted with CH₂Cl₂, dried over Na₂SO₄ and concentrated to give compound M-6. ESI-MS calc. for C₁₇H₃₃N₃O₂: 311. Found: 312 (M+H).

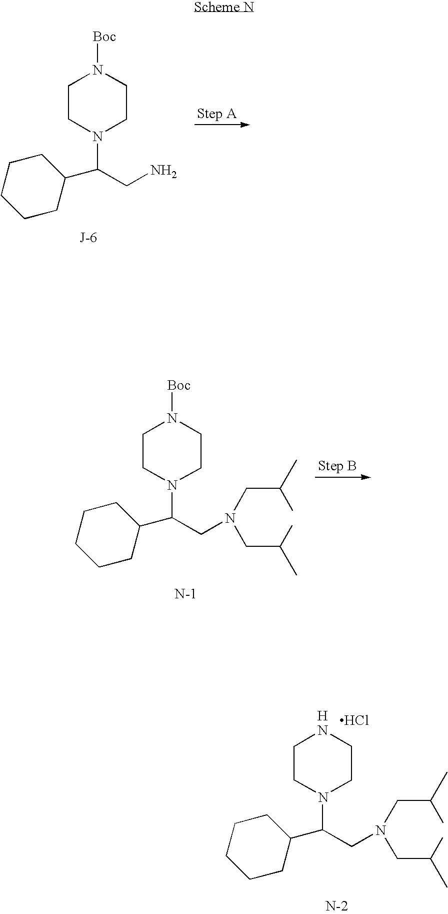

Step A: To a solution of Intermediate J-6 (0.67 g, 2.15 mmol) in CH₂Cl₂ (10 mL) was added isobutylaldehyde (0.59 mL, 6.46 mmol), and acetic acid (0.25 mL, 4.3 mmol). After stirring the mixture for 10 minutes, sodium triacetoxyborohydride (1.83 g, 8.62 mmol) was added. The reaction mixture was stirred at room temperature for 3.5 hr, then diluted with CH₂Cl₂, washed with saturated NaHCO₃, extracted the aqueous with CH₂Cl₂. The combined organic layers were washed with brine, dried over Na₂SO₄ and concentrated to give a crude product. The crude product was purified by chromatography over silica gel (hexane:ethyl acetate=2:1 to ethyl acetate) to give Intermediate N-1. ESI-MS calc. for C₂₅H₄₉N₃O₂: 423. Found: 424 (M+H)
Step B: To the intermediate N-1 (0.45 g) was added hydrogen chloride (4.0 Min dioxane). The reaction mixture was stirred at room temperature for 30 minutes, then the solvent was removed in vacuo to afford intermediate N-2. ESI-MS calc. for C₂₀H₄₁N₃: 323. Found: 324 (M+H).

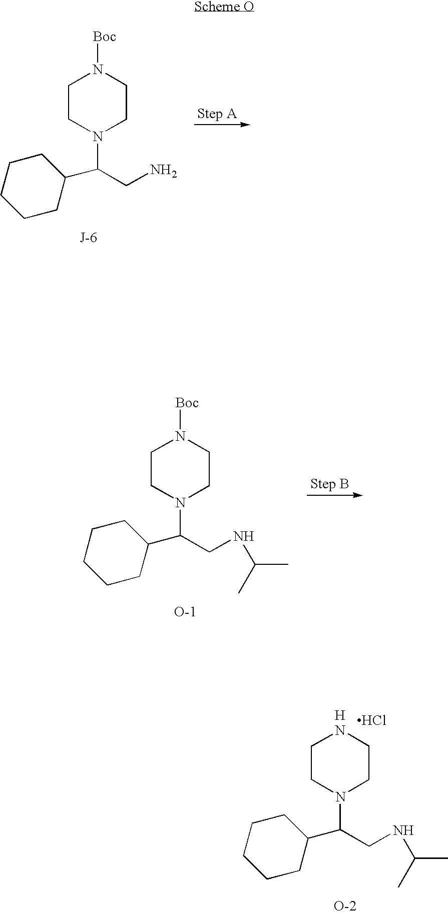

Step A: Intermediate O-1 was prepared from intermediate J-6 following a procedure analogous to the procedure described for the preparation of Intermediate N-1. ESI-MS calc. for C₂₀H₃₉N₃O₂: 353. Found: 354 (M+H).
Step B: To the intermediate O-1 (0.45 g) was added hydrogen chloride (4.0 M in dioxane). The reaction mixture was stirred at room temperature for 30 minutes, then the solvent was removed in vacuo to afford intermediate O-2. ESI-MS calc. for C₁₅H₃₁N₃: 253. Found: 254 (M+H).

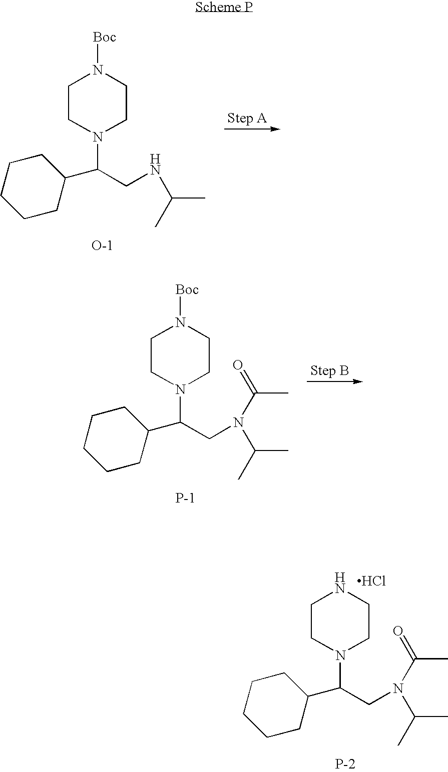

Step A: To a solution of Intermediate O-1 (482 mg, 1.365 mmol) in pyridine (10 mL) was added acetic anhydride (0.13 mL, 1.38 mmol). The reaction mixture was stirred at room temperature for 18 hours, then concentrated to give a residue. The residue was dissolved in CH₂Cl₂, washed with water, brine, dried over Na₂SO₄ and concentrated to give a crude product, which was purified by chromatography over silica gel (hexane:ethyl acetate=2:1 to hexane:ethyl acetate=1:1) to give Intermediate P-1. ESI-MS calc. for C₂₂H₄₁N₃O₃: 395.6. Found: 396 (M+H), 418 (M+Na)
Step B: Intermediate P-2 was prepared from Intermediate P-1 following a procedure analogous to the procedure described for the preparation of Intermediate O-2. ESI-MS 296 (m+1).

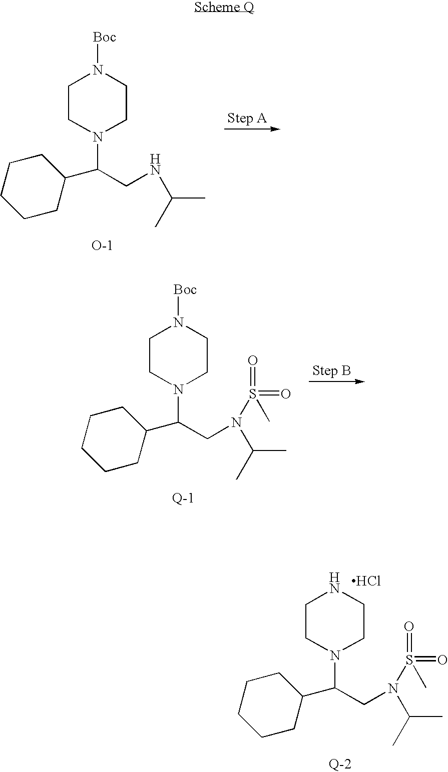

Step A: To a solution of Intermediate O-1 (500 mg, 1.42 mmol) in CH₂Cl₂ (20 mL) was added triethylamine (0.40 mL, 2.83 mmol) and methansulfonyl chloride (0.12 mL, 1.56 mmol). The mixture was stirred at room temperature for 18 hours, then concentrated to give a residue. The residue was dissolved in CH₂Cl₂, washed with water, brine, dried over Na₂SO₄ and concentrated to give Intermediate Q-1. ESI-MS calc. for C₂₁H₄₁N₃O₄S: 431.3. Found: 432 (M+H).
Step B: Intermediate Q-2 was prepared from Intermediate Q-1 following a procedure analogous to the procedure described for the preparation of Intermediate O-2. ESI-MS calc. for C₁₆H₃₃N₃O₂S: 331. Found: 332 (M+H).

The following Examples are provided to illustrate the invention and are not to be construed as limiting the scope of the invention in any manner.

### Example 1

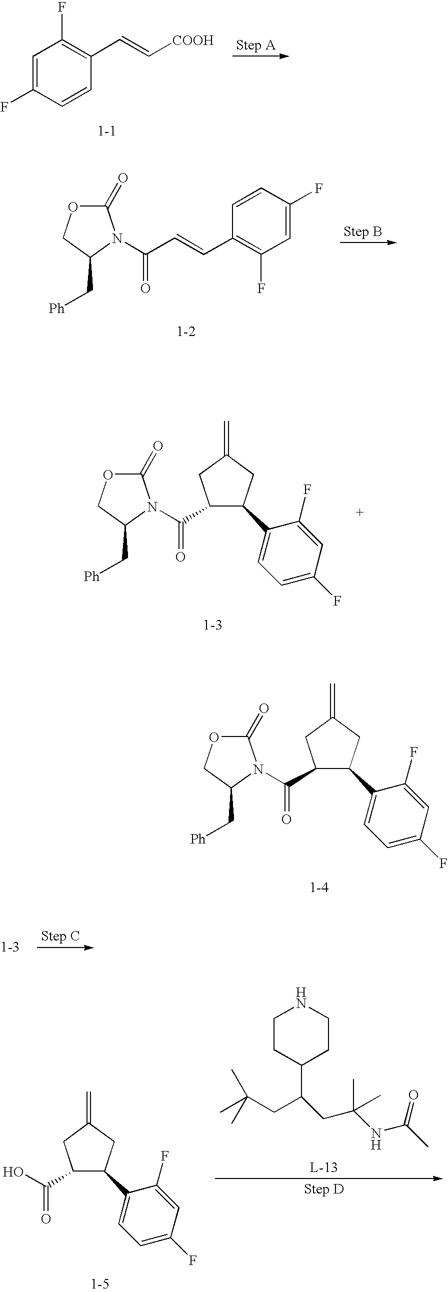

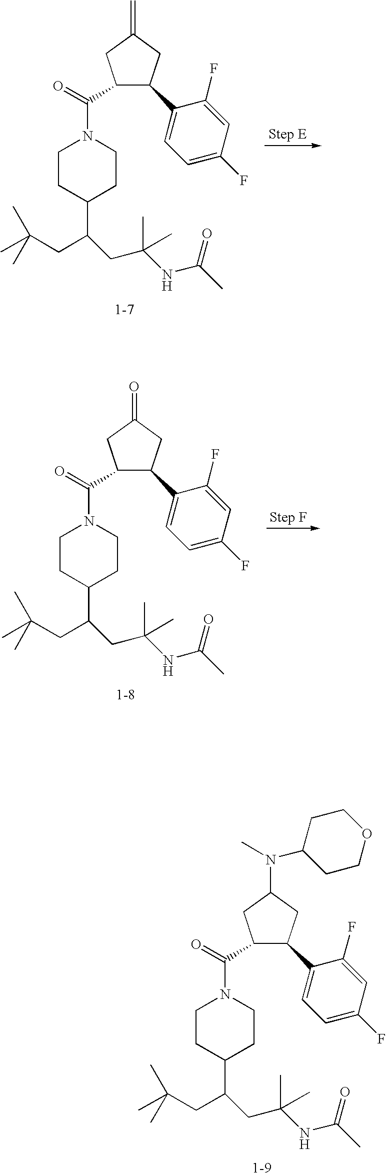

Step A: To a solution of trans-2, 4-difluorocinnamic acid 1-1 (7.6 g, 41.3 mmol, Aldrich) in THF (150 mL) was added triethylamine (17.3 mL, 123.8 mmol). The reaction mixture was cooled to −40° C. and trimethyl acetic chloride (5.1 mL, 47.3 mmol) was added slowly. After the reaction mixture was stirred at −40° C. for another 2 hours, the lithium chloride (1.93 g, 45.40 mmol) was added, followed by s-4-benzyl-2-oxazolidinone (7.31 g, 41.3 mmol). After stirring at −40° C. for another 20 min., reaction mixture was allowed to warm up to room temperature and stirred at r.t. for 18 hrs. The reaction mixture was poured into aqueous of saturated ammonium chloride (180 mL); the phases were separated and the aqueous phase was extracted with ethyl acetate. The combined ethyl acetate extracts were washed with brine, dried over MgSO₄ and concentrated to give a residue. The resulting residue was purified by crystallization from EtOAc/hexane to give compound 1-2. ESI-MS calc. for C₁₉H₁₅F₂NO3: 343. Found: 344 (M+H), 366 (M+Na).
Step B: To a solution of Compound I-2 (2.3 g, 6.55 mmol) in THF (30 mL) was added palladium acetate (73.6 mg, 0.33 mmol) and 2-[(trimethylsilyl)methyl]-2-propenol-yl acetate (1.8 mL, 8.52 mmol). The reaction vessel was evacuated under vacuum and purged with nitrogen 3 times, then triisopropyl phosphate (0.45 mL, 1.97 mmol) was added. The reaction mixture was heated at 65° C. for 18 hrs, cooled to r.t. and concentrated to give a residue. The resulting residue was partitioned between ethyl acetate and water, and the aqueous layer was extracted with ethyl acetate. The combined extracts were washed with brine, dried over MgSO₄ and concentrated to give a residue. The resulting residue was purified by HPFC (2-30% ethyl acetate in hexane) to give a yellow oil 1-4 (0.89 g, fast elusion) and white solid 1-3 (0.85 g, slow elusion). ESI-MS calc. for C₂₃H₂₁F₂NO₃: 397. Found: 398 (M+H), 420 (M+Na).
Step C: To a solution of Compound I-3 (1.7 g, 4.28 mmol) in THF (24 mL) and water (6 mL) under nitrogen at 0° C. was added lithium hydroxide monohydrate (0.36 g, 8.56 mmol) and H₂O₂ (30% solution, 2.5 mL, 25.7 mmol). After stirring the reaction mixture at 0° C. for half an hour, the mixture was warmed up to r.t. and stirred for 1.5 hours. The solvent was removed, the pH was adjusted to pH 9-10 with a saturated NaHCO₃ solution and the mixture was extracted with CH₂Cl₂. The aqueous layer was acidified with HCl (2N) to pH 1-2, and the mixture was extracted with CH₂Cl₂. The combined methylene chloride layers were dried over MgSO₄ and concentrated to give colorless oil 1-5. ESI-MS calc. for C₁₃H₁₂F₂O₂: 238. Found: 239 (M+H).
Step D: To a solution of compound I-5 (0.41 g, 1.73 mmol) in dichloromethane (30 mL) was added NMM (0.26 mL, 2.36 mmol), HOBt (0.23 g, 1.73 mmol), EDC (0.45 g, 2.36 mmol) and amine L-13 (0.50 g, 1.58 mmol). The reaction mixture was stirred at room temperature overnight, diluted with dichloromethane, and washed with water and brine. The organic layer was dried over anhydrous magnesium sulfate, filtered, and concentrated to give a residue. The resulting residue was purified by HPFC (20%-100% EtOAc in hexane) to give compound I-7. ESI-MS calc. for C₃₀H₄₄F₂N₂O₂: 502. Found: 503 (M+H).
Step E: To a solution of Compound I-7 (0.35 g, 0.70 mmol) in THF (10 mL) and water (10 mL) at room temperature was added OsO₄ (2.5 wt % solution in t-BuOH, 0.87 mL, 0.070 mmol). After stirring the reaction mixture at r.t. for 10 minutes, sodium periodate (0.497 g in 4.5 mL H₂O, 2.32 mmol) was added slowly over 15 minutes, and the mixture was stirred for 1.5 hrs. Then sodium thiosulfate pentahydrate (2.1 mmol, 0.52 g, sat) was added and the reaction mixture was stirred for an additional 15 minutes. The layers were separated, the aqueous layer was extracted with EtOAc, dried over MgSO₄, filtered and concentrated to give 1-8 (0.35 g) as a black solid. ESI-MS calc. for C₂₉H₄₂F₂O₃: 504. Found: 505 (M+H).
Step F: To a solution of N-tetrahydropyran methylamine hydrochloric acid salt (34 mg, 0.225 mmol) in dichloromethane (2 mL) was added triethylamine (0.0766 mL, 0.55 mmol). After stirred at room temperature for 10 minutes, Compound I-8 (25 mg, 0.050 mmol) and acetic acid (0.021 mL, 0.36 mmol) were added. The reaction mixture was stirred at room temperature for 10 minutes, followed by the addition of sodium triacetoxyborohydride (84 mg, 0.40 mmol). After stirring for 18 hours, the reaction mixture was diluted with CH₂Cl₂, washed with saturated NaHCO₃ and brine, dried over anhydrous sodium sulfate, filtered and concentrated to give a residue. The resulting residue was separated by prep TLC (CHCl₃:2N NH₃ in CH₃OH=10:1) to give compound I-9. ESI-MS calc. for C₃₅H₅₅F₂N₃O₃: 604. Found: 605 (M+H). ¹H NMR (500 HMz, CD₃OD): 7.6-7.4 (m, 1H), 7.0-6.8 (m, 2H), 4.6-4.5 (m, 1H), 4.0-3.9 (m, 2H), 3.7-3.6 (m, 1H), 3.5-3.4 (m, 2H), 2.95-2.75 (m, 1H), 2.5-2.38 (m, 1H), 2.3 (s, 3H), 2.27-2.05 (m, 2H), 2.05-1.9 (m, 3H), 1.85 (s, 3H), 1.8-1.6 (m, 5H), 1.6-1.4 (m, 2H), 1.4-1.2 (m, 10H), 1.0-0.9 (m, 15H), 0.25-0.15 (m, 1H).

Examples 2 to 4 were prepared from appropriate amine and intermediate 1-8 in an analogous manner to the synthesis described in Example 1, Step F.

| | | | Parent Ion |
|---|---|---|---|
| | | Calculated | m/z (M + H) |
| Example | R¹ | MW | ESI-MS |
| 2 | 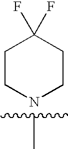 | C₃₄H₅₁F₄N₃O₂ 610 | 611 |
| 3 | 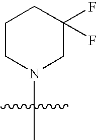 | C₃₄H₅₃F₂N₃O₃ 590 | 591 |
| 4 | 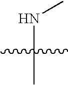 | C₃₀H₄₇F₂N₃O₂ 519 | 520 |

Examples 5 to 13 were prepared from appropriate aldehyde and compound 4 in an analogous manner to the synthesis described in Example 1, Step F.

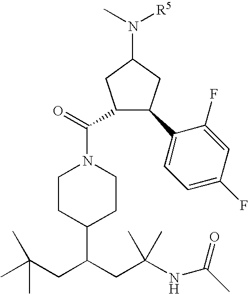

| | | | Parent Ion |
|---|---|---|---|
| | | Calculated | m/z (M + H) |
| Example | R⁵ | MW | ESI-MS |
| 5 | 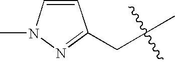 | C₃₅H₅₃F₂N₅O₂ 614 | 615 |
| 6 | 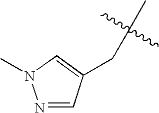 | C₃₅H₅₃F₂N₅O₂ 614 | 615 |
| 7 | 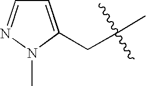 | C₃₅H₅₃F₂N₅O₂ 614 | 615 |
| 8 | 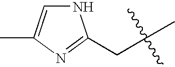 | C₃₅H₅₃F₂N₅O₂ 614 | 615 |
| 9 | 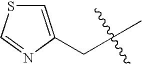 | C₃₄H₅₀F₂N₄O₂S 617 | 618 |
| 10 | 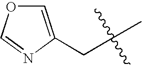 | C₃₄H₅₀F₂N₄O₃ 601 | 602 |
| 11 | 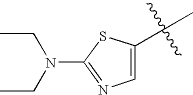 | C₃₈H₅₉F₂N₅O₂S 688 | 689 |
| 12 | 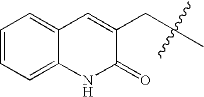 | C₄₀H₅₄F₂N₄O₃ 677 | 678 |
| 13 | 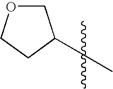 | C₃₄H₅₃F₂N₃O₃ 590 | 591 |

### Example 14

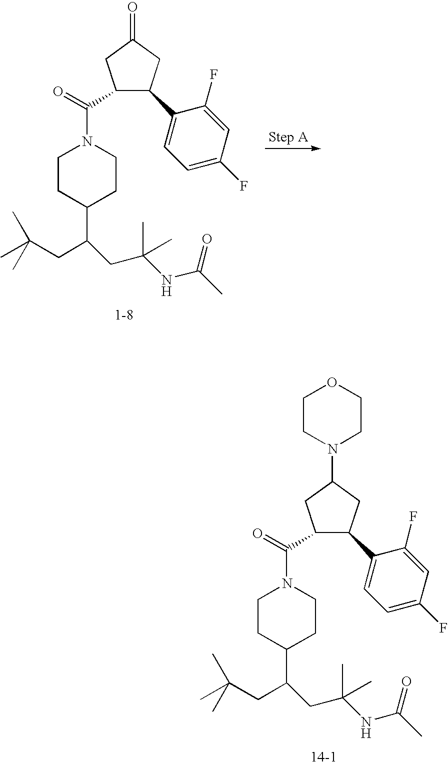

Step A: Compound 14-1 was prepared from intermediate 1-8 and morpholine following a procedure analogous to the procedure described for step F of Example 1. ESI-MS calculated for Compound 14-1: C₃₃H₅₁F₂N₃O₃: 576. Found: 577 (M+H).

### Example 15

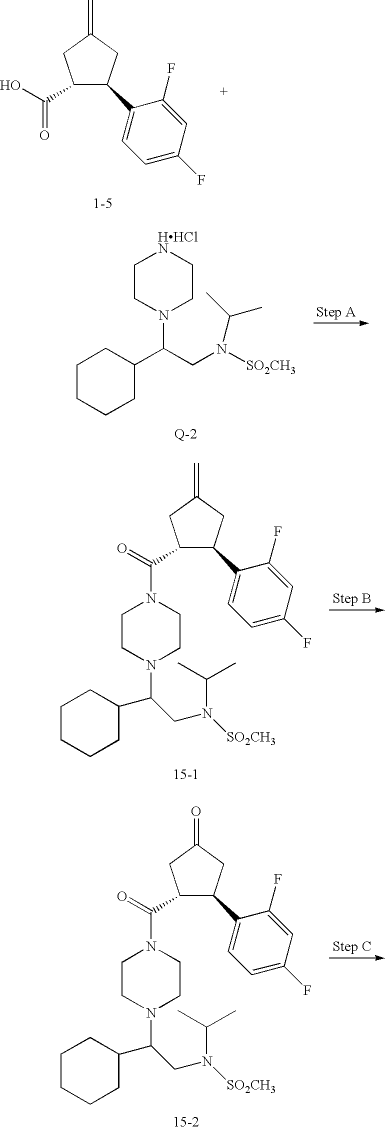

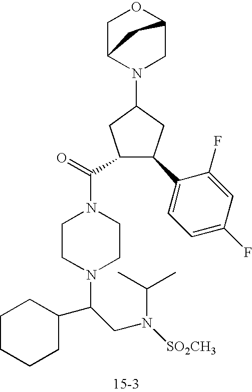

Step A: Amide 15-1 was prepared from alkene 1-5 and piperazine Q-2 following a procedure analogous to the procedure described for step D of Example 1. ESI-MS calculated for amide 15-1: C₂₉H₄₃F₂N₃O₃S: 551. Found: 552 (M+H).
Step B: Ketone 15-2 was prepared from amide alkene 15-1 following a procedure analogous to the procedure described for step E of Example 1. ESI-MS calculated for ketone 15-2: C₂₈H₄₁F₂N₃O₄S: 553. Found: 554 (M+H).
Step C: Compound 15-3 was prepared from intermediate 15-2 and 2-oxa-5-azabicyclo [2,2,1]heptane hydrochloride following a procedure analogous to the procedure described for step F of Example 1. ESI-MS calculated for Compound 15-3: C₃₃H₅₀F₂N₄O₄S: 636. Found: 637 (M+H). ¹H NMR (500 HMz, CD₃OD): 7.5-7.35 (m, 1H), 7.0-6.8 (m, 2H), 4.5-4.4 (m, 1H), 4.1-4.0 (m, 1H), 3.9-3.7 (m, 3H), 3.7-3.6 (m, 2H), 3.6-3.4 (m, 2H), 3.4-3.2 (m, 4H), 3.2-3.1 (m, 11-1), 3.1-2.9 (m, 1H), 2.95 (s, 3H), 2.9-2.4 (m, 4H), 2.4-2.1 (m, 3H), 2.1-1.9 (m, 3H), 1.9-1.6 (m, 6H), 1.6-1.4 (m, 1H), 1.4-1.2 (m, 9H), 1.2-1.0 (m, 3H).

Examples 16 to 17 were prepared from appropriate amine and intermediate 13-3 following the procedure described in Step F of Example 1.

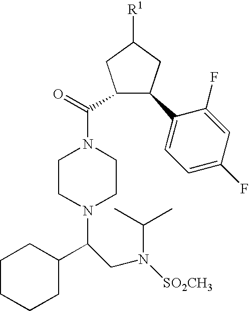

| | | | Parent Ion |
|---|---|---|---|
| | | Calculated | m/z (M + H) |
| Example | R¹ | MW | ESI-MS |
| 16 | 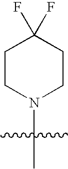 | C₃₃H₅₀F₄N₄O₃S 658 | 659 |
| 17 | 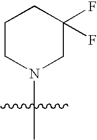 | C₃₃H₅₀F₄N₄O₃S 658 | 659 |

### Biological Assays

### A. Binding Assay

The membrane binding assay was used to identify competitive inhibitors of ¹²⁵I-NDP-alpha-MSH binding to cloned human MCRs expressed in mouse L- or Chinese hamster ovary (CHO)-cells.

Cell lines expressing melanocortin receptors were grown in T-180 flasks containing selective medium of the composition: 1 L Dulbecco's modified Eagles Medium (DMEM) with 4.5 g L-glucose, 25 mM Hepes, without sodium pyruvate, (Gibco/BRI); 100 mL 10% heat-inactivated fetal bovine serum (Sigma); 10 mL 10,000 unit/mL penicillin & 10,000 μg/mL streptomycin (Gibco/BRI); 10 mL 200 mM L-glutamine (Gibco/BRI); 1 mg/mL geneticin (G418) (Gibco/BRI). The cells were grown at 37° C. with CO₂ and humidity control until the desired cell density and cell number was obtained.

The medium was poured off and 10 mL/monolayer of enzyme-free dissociation media (Specialty Media Inc.) was added. The cells were incubated at 37° C. for 10 mM or until cells sloughed off when flask was banged against hand.

The cells were harvested into 200 mL centrifuge tubes and spun at 1000 rpm, 4° C., for 10 mM. The supernatant was discarded and the cells were resuspended in 5 mL/monolayer membrane preparation buffer having the composition: 10 mM Tris pH 7.2-7.4; 4 μg/mL Leupeptin (Sigma); 10 μM Phosphoramidon (Boehringer Mannheim); 40 μg/mL Bacitracin (Sigma); 5 μg/mL Aprotinin (Sigma); 10 mM Pefabloc (Boehringer Mannheim). The cells were homogenized with motor-driven dounce (Talboy setting 40), using 10 strokes and the homogenate centrifuged at 6,000 rpm, 4° C., for 15 mM.

The pellets were resuspended in 0.2 mL/monolayer membrane prep buffer and aliquots were placed in tubes (500-1000 μL/tube) and quick frozen in liquid nitrogen and then stored at −80° C.

Test compounds or unlabelled NDP-α-MSH was added to 100 μL of membrane binding buffer to a final concentration of 1 μM. The membrane binding buffer had the composition: 50 mM Tris pH 7.2; 2 mM CaCl₂; 1 mM MgCl₂; 5 mM KCl; 0.2% BSA; 4 μg/mL Leupeptin (SIGMA); 10 μM Phosphoramidon (Boehringer Mannheim); 40 μg/mL Bacitracin (SIGMA); 5 μg/mL Aprotinin (SIGMA); and 10 mM Pefabloc (Boehringer Mannheim). One hundred μL of membrane binding buffer containing 10-40 μg membrane protein was added, followed by 100 μM 125I-NDP-α-MSH to final concentration of 100 μM. The resulting mixture was vortexed briefly and incubated for 90-120 mM at room temp while shaking.

The mixture was filtered with Packard Microplate 196 filter apparatus using Packard Unifilter 96-well GF/C filter with 0.1% polyethyleneimine (Sigma). The filter was washed (5 times with a total of 10 mL per well) with room temperature of filter wash having the composition: 50 mM Tris-HCl pH 7.2 and 20 mM NaCl. The filter was dried, and the bottom sealed and 50 μL of Packard Microscint-20 was added to each well. The top was sealed and the radioactivity quantitated in a Packard Topcount Microplate Scintillation counter.

### B. Functional assay

Functional cell based assays were developed to determine the efficacy of agonists and to discriminate melanocortin receptor agonists from antagonists.

Cells (for example, CHO- or L-cells or other eukaryotic cells) expressing a human melanocortin receptor (see e.g. Yang-Y K; Ollmann-M M; Wilson-B D; Dickinson-C; Yamada-T; Barsh-G S; Gantz-I; Mol-Endocrinol. 1997 March; 11(3): 274-80) were dissociated from tissue culture flasks by rinsing with Ca and Mg free phosphate buffered saline (14190-136, Life Technologies, Gaithersburg, Md.) and detached following 5 mM incubation at 37° C. with enzyme free dissociation buffer (S-014-B, Specialty Media, Lavellette, N.J.). Cells were collected by centrifugation and resuspended in Earle's Balanced Salt Solution (14015-069, Life Technologies, Gaithersburg, Md.) with additions of 10 mM HEPES pH 7.5, 5 mM MgCl₂, 1 mM glutamine and 1 mg/mL bovine serum albumin. Cells were counted and diluted to 1 to 5×10⁶/mL. The phosphodiesterase inhibitor 3-isobutyl-1-methylxanthine was added to cells to 0.6 mM.

1. Agonist Assay Test compounds were diluted in dimethylsulfoxide (DMSO) (10⁻⁵ to 10⁻¹⁰ M) and 0.1 volume of compound solution was added to 0.9 volumes of cell suspension; the final DMSO concentration was 1%. After room temperature incubation for 45 mM, cells were lysed by incubation at 100° C. for 5 min to release accumulated cAMP. cAMP was measured in an aliquot of the cell lysate with the Amersham (Arlington Heights, Ill.) cAMP detection assay (RPA556). The amount of cAMP production which resulted from an unknown compound was compared to that amount of cAMP produced in response to alpha-MSH which was defined as a full agonist with an efficacy of 100%. The EC₅₀ is defined as the compound concentration which results in half maximal stimulation, when compared to its own maximal level of stimulation. Compounds that produce near 0% response are expected to be antagonist which will be further confirmed in the antagonist mode of the functional assay.

2. Antagonist Assay: Antagonist activity was defined as the ability of a compound to block cAMP production in response to alpha-MSH or any agonist. A solution of the test compound and suspension of receptor containing cells were prepared and mixed as described above; the mixture was incubated for 15 min, and an EC₅₀ dose of alpha-MSH (approximately 10 nM alpha-MSH) was added to the cells. The assay was terminated at 45 minutes and cAMP quantitated as above. Percent inhibition was determined by comparing the amount of cAMP produced in the presence to that produced in the absence of test compound. Antagonist is defined as a compound that by itself does not produce agonist-like response, and in combination with an agonist the compound should inhibit the agonist-induced response.

### C. In Vivo Food Intake and Body Weight Models

1) Food intake and body weight in rats. Sprague Dawley rats are administered test compound one hour prior to onset of dark cycle (12 hours). Food intake is determined either by measurement of the remaining amount of preweighed food the morning following the dosing or by using a computerized system in which each rat's food is placed on a computer monitored balance. Cumulative food intake for 16 h post compound administration is measured. In some cases, food intake measurements are followed as long as 2 weeks. Body weight is measured daily; in some cases, adiposity is measured by DEXAscan analysis, tissue weights and plasma drug levels are measured. Animals can be dosed by a number of routes of administration. The routes of administration include intravenous (IV), intraperitoneal (IP), subcutaneous (SC) and intracerebral ventricular (Icy).

Compounds useful in the present invention decrease food intake acutely by at least 20% and/or decrease body weight in a 2 week period by at least 4% relative to placebo.

2) Food intake in diet induced obese mice. Male C57/B16J mice maintained on a high fat diet (30-60% fat calories) are dosed with test compound for 1 to 30 days. Food intake and body weight are measured overnight and sometimes daily as long as 30 days. Biochemical parameters relating to obesity, including leptin, insulin, triglyceride, free fatty acid, cholesterol and serum glucose levels and pharmacokinetic parameters may be determined. Animals can be dosed by a number of routes of administration. The routes of administration include intravenous, intraperitoneal, subcutaneous and intracerebral ventricular. Biochemical parameters relating to obesity, including leptin, insulin, triglyceride, free fatty acid, cholesterol and serum glucose levels are determined.

Compounds useful in the present invention decrease body weight by at least 4% relative to placebo.

### D. Male Sexual Dysfunction

### Mouse Electrically Stimulated Cavernosal Nerve (ESCN) Assay

Male C57BL6 mice are anesthetized, the carotid artery is exposed and cannulated for measurement of arterial pressure (MAP). A 30G needle attached to PE 10 tubing, filled with heparinized saline, was inserted into the artery and glued in place. This tubing was connected to a pressure transducer and amplifier to measure direct MAP on a Gould 8 channel oscilloscope connected to a computer using the Po-ne-mah software to collect the data at one minute intervals. Another PE10 line attached to a 30G needle was inserted into the jugular vein for compound or vehicle administration. The cavernous nerve and penile body were exposed through a midline incision. Surrounding muscles were cauterized and removed for visualization of the cavernous nerve, which arises from the ipsilateral pelvic ganglion and is situated dorsal to the prostate. Another 30G needle attached to PE10 tubing, filled with heparinized saline, was inserted into the base of the corpus cavernosum near the crura and connected to the Gould system. A slight increase in intercavernous pressure (ICP) of approximately 5 to 10 mmHg is observed once this cannula is inserted into the corpus cavernosum. Heparinized saline (200 units/mL) was flushed through the cannula to assure proper placement of the cannula, inducing tumescence. The cavernous nerve was then isolated using curved #5 Dumont forceps and placed on a modified fixed position bipolar silver electrode (Harvard Apparatus). The electrodes are encased in plastic to allow stimulation of the nerve without additional stimulation of surrounding tissues. The electrode was advanced and held by a micromanipulator and was attached to a square wave stimulator to deliver electrical impulses at stimulation parameters ranging between 0.5 to 6.0v, 2 to 16 Hz, 1 ms, for 30 seconds. Electrical stimulations were administered to individual animals with 5 minute intervals between stimulations. Responses reported at each time point represent the mean of the two stimulations. ICP, MAP and ICP/MAP responses were continuously recorded at one second intervals for the duration of the experiment.

Measurements of ICP, MAP and ICP/MAP ratio are analyzed and responses compared to nerve stimulation in the presence and absence of compound or vehicle. For each parameter monitored, responses evoked by duplicate electrical stimulations were averaged, and the mean values were used for comparison. Response segments of 10 s of baseline+30 s stimulation+150 s post-stimulation were used to evaluate changes in ICP in response to electrical stimulation of the cavernous nerve. To assess direct effects of compound administration on ICP, a 300 s pre-compound response segment was compared to a comparable segment immediately after compound administration.

Compounds useful in the present invention increase intracavernous pressure by at least 25% for a time period of at least 15 minutes relative to placebo.

### E. Models of Female Sexual Dysfunction

Rodent assays relevant to female sexual receptivity include the behavioral model of lordosis and direct observations of copulatory activity. There is also an urethrogenital reflex model in anesthetized spinally transected rats for measuring orgasm in both male and female rats. These and other established animal models of female sexual dysfunction are described in McKenna K E et al, A Model For The Study of Sexual Function In Anesthetized Male And Female Rats, Am. J. Physiol. (Regulatory Integrative Comp. Physiol 30): R1276-R1285, 1991; McKenna K E et al, Modulation By Peripheral Serotonin of The Threshold For Sexual Reflexes In Female Rats, Pharm. Bioch. Behay., 40:151-156, 1991; and Takahashi L K et al, Dual Estradiol Action In The Diencephalon And The Regulation Of Sociosexual Behavior In Female Golden Hamsters, Brain Res., 359:194-207, 1985.

### F. Model of Cachexia

Rodent assays relevant to cachexia include the tumor cachexia model, in which cells derived from a tumor were injected into mice. Over a period of 1-3 weeks, a tumor will form and grow in the implanted mice. Tumor-bearing mice will exhibit reduced food intake and reduced body weight. By treating the tumor-bearing mice with an effective MC4R antagonist, food intake will be increased and body weight will be increased. This animal model of cachexia is described in Cone, R. D. et al, Role of the Central Melanocortin System in Cachexia, Cancer Research 61, 1432-38, Feb. 15, 2001.

Representative compounds of the present invention were tested and found to bind to the melanocortin-4 receptor. These compounds were generally found to have IC₅₀ values less than 10 μM. Representative agonist compounds of the present invention were also tested in the functional assay and found generally to activate the melanocortin-4 receptor with EC₅₀ values less than 5 μM.

Representative antagonist compounds of the present invention were tested in the functional assay and found generally not to activate the melanocortin-4 receptor with an efficacy <5%, and generally have an IC₅₀ from the antagonist assay of less than 10 uM.

### Examples of Pharmaceutical Compositions

As a specific embodiment of an oral composition of a composition of the present invention, 5 mg of Example 1 is formulated with sufficient finely divided lactose to provide a total amount of 580 to 590 mg to fill a size O hard gelatin capsule.

As another specific embodiment of an oral composition of a compound of the present invention, 2.5 mg of Example 1 is formulated with sufficient finely divided lactose to provide a total amount of 580 to 590 mg to fill a size O hard gelatin capsule.

While the invention has been described and illustrated in reference to certain preferred embodiments thereof, those skilled in the art will appreciate that various changes, modifications and substitutions can be made therein without departing from the spirit and scope of the invention. For example, effective dosages other than the preferred doses as set forth hereinabove may be applicable as a consequence of variations in the responsiveness of the subject or mammal being treated for severity of bone disorders caused by resorption, or for other indications for the compounds of the invention indicated above. Likewise, the specific pharmacological responses observed may vary according to and depending upon the particular active compound selected or whether there are present pharmaceutical carriers, as well as the type of formulation and mode of administration employed, and such expected variations or differences in the results are contemplated in accordance with the objects and practices of the present invention. It is intended, therefore, that the invention be limited only by the scope of the claims which follow and that such claims be interpreted as broadly as is reasonable.

1. A compound of structural formula I: 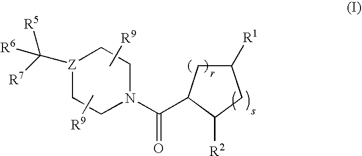
or a pharmaceutically acceptable salt thereof; wherein:
Z is N or CR⁴;
R¹ is selected from the group consisting of:
(1) amidino,
(2) —C₁₋₄alkyliminoyl,
(3) —C₁₋₈ alkyl,
(4) —(CH₂)ₙN(R⁸)₂,
(5) —(CH₂)ₙC₂₋₉heterocycloalkyl,
(6) —(CH₂)ₙC₃₋₈cycloalkyl,
(7) —(CH₂)ₙphenyl,
(8) —(CH₂)ₙnaphthyl,
(9) —(CH₂)ₙheteroaryl,
(10) —(CH₂)ₙC(O)C₁₋₈ alkyl,
(11) —(CH₂)ₙC(O)C₃₋₈cycloalkyl,
(12) —(CH₂)ₙC(O)C₂₋₉heterocycloalkyl,
(13) —(CH₂)ₙC(O)phenyl,
(14) —(CH₂)ₙC(O)naphthyl,
(15) —(CH₂)ₙC(O)heteroaryl,
(16) —(CH₂)ₙCO₂H,
(17) —(CH₂)ₙCO₂C₁₋₈ alkyl,
(18) —(CH₂)ₙCO₂C₃₋₈cycloalkyl,
(19) —(CH₂)ₙCO₂C₂₋₉heterocycloalkyl,
(20) —(CH₂)ₙCO₂-phenyl,
(21) —(CH₂)ₙCO₂naphthyl,
(22) —(CH₂)ₙCO₂heteroaryl,
wherein phenyl, naphthyl, and heteroaryl are unsubstituted or substituted with one to three substituents independently selected from R³, and alkyl, cycloalkyl, heterocycloalkyl and (CH₂)ₙ are unsubstituted or substituted with one to three substituents independently selected from R³ and oxo;
R² is selected from the group consisting of:
(1) phenyl,
(2) naphthyl, and
(3) heteroaryl,
wherein phenyl, naphthyl, and heteroaryl are unsubstituted or substituted with one to three substituents independently selected from R¹⁰;
each R³ is independently selected from the group consisting of:
(1) —C₁₋₈ alkyl,
(2) —(CH₂)ₙ-phenyl,
(3) —(CH₂)ₙ-heteroaryl,
(4) —(CH₂)ₙC₂₋₉heterocycloalkyl,
(5) —(CH₂)ₙC₃₋₇ cycloalkyl,
(6) halogen,
(7) —OR⁸,
(8) —(CH₂)ₙCE-N,
(9) —(CH₂)ₙN(R⁸)₂,
(10) —(CH₂)ₙC(O)NR⁸)₂,
(11) —(CH₂)ₙC(O)NR⁸N(R⁸)₂,
(12) —(CH₂)ₙC(O)NR⁸NR⁸C(O)R⁸, and
(13) —(CH₂)ₙCF₃,
wherein phenyl and heteroaryl are unsubstituted or substituted with one to three substituents independently selected from halogen, hydroxy, C₁₋₄ alkyl, trifluoromethyl, and C₁₋₄ alkoxy, and wherein any alkyl, cycloalkyl, heterocycloalkyl, and methylene (CH₂)ₙ carbon atom in R³ is unsubstituted or substituted with one to two substituents independently selected from halogen, hydroxy, oxo, C₁₋₄ alkyl, trifluoromethyl, and C₁₋₄ alkoxy, or two R³ substituents on the same carbon atom are taken together with the carbon atom to form a cyclopropyl group;
R⁴ is selected from the group consisting of:
(1) hydrogen,
(2) —C₁₋₆ alkyl, and
(3) —OC₁₋₆ alkyl;
R⁵ is selected from the group consisting of:
(1) —CF₃,
(2) —C₁₋₆ alkyl,
(3) —C₂₋₈ alkenyl,
(4) —C₂₋₈ alkynyl,
(5) —OC₁₋₈ alkyl,
(6) —(CH₂)ₙC₃₋₈cycloalkyl,
(7) —(CH₂)ₙC₂₋₉heterocycloalkyl,
(8) —(CH₂)ₙ-phenyl,
(9) —(CH₂)ₙ-naphthyl, and
(10) —(CH₂)ₙheteroaryl,
wherein phenyl, naphthyl, and heteroaryl are unsubstituted or substituted with one to three substituents independently selected from R³, and alkyl, alkenyl, alkynyl, cycloalkyl and heterocycloalkyl are unsubstituted or substituted with one to three substituents independently selected from R³ and oxo, and wherein any methylene (CH₂) in R⁵ is unsubstituted or substituted with one to two substituents independently selected from halogen, hydroxy, oxo, and C₁₋₄ alkyl;
R⁶ is selected from the group consisting of:
(1) hydrogen,
(2) —C₁₋₆ alkyl, and
(3) —OC₁₋₆ alkyl;
R⁷ is selected from the group consisting of:
(1) —(CH₂)ₙN(R⁸)₂,
(2) —(CH₂)ₙNR⁸C(O)R⁸,
(3) —(CH₂)ₙOR⁸,
(4) —(CH₂)ₙC≡N,
(5) —(CH₂)ₙC(O)OR⁸,
(6) —(CH₂)ₙC(O)N(R⁸)₂,
(7) —(CH₂)ₙNR⁸C(O)N(R⁸)₂,
(8) —(CH₂)ₙNR⁸C(O)heteroaryl,
(9) —(CH₂)ₙheteroaryl,
(10) —(CH₂)ₙNR⁸S(O)ₚR⁸,
(11) —(CH₂)ₙSR⁸, and
(12) —(CH₂)ₙS(O)ₚR⁸,
wherein heteroaryl is unsubstituted or substituted with one to three substituents selected from C₁₋₄ alkyl, and any methylene (CH₂) in R⁷ is unsubstituted or substituted with one to two substituents independently selected from halogen, hydroxy, oxo, and C₁₋₄ alkyl, or two C₁₋₄ alkyl substituents on any methylene (CH₂) in R⁷ together with the atom to which they are attached form a 3, 4, 5, or 6-membered ring optionally containing an additional heteroatom selected from O, S, —NH, and —NC₁₋₄ alkyl;
each R⁸ is independently selected from the group consisting of:
(1) hydrogen,
(2) —C₁₋₈ alkyl,
(3) —C₂₋₈ alkenyl,
(4) —C₂₋₈ alkynyl,
(5) —OC₁₋₈ alkyl,
(6) —(CH₂)ₙC₃₋₈cycloalkyl,
(7) —(CH₂)ₙC₂₋₉heterocycloalkyl,
(8) —(CH₂)ₙ-phenyl,
(9) —(CH₂)ₙ-naphthyl, and
(10) —(CH₂)ₙheteroaryl,
wherein phenyl, naphthyl, and heteroaryl, alkyl, alkenyl, alkynyl, cycloalkyl and heterocycloalkyl are unsubstituted or substituted with one to three substituents independently selected from —N(C₁₋₆alkyl)₂, —NH₂, NH(C₁₋₆ alkyl), halogen, C₁₋₆alkyl, C₁₋₆alkoxy, hydroxy, and oxo, and wherein any methylene (CH₂) in R⁸ is unsubstituted or substituted with one to two substituents independently selected from halogen, hydroxy, oxo, and C₁₋₄ alkyl;
each R⁹ is independently selected from the group consisting of:
(1) hydrogen,
(2) —OH,
(3) C₁₋₈alkyl,
(4) —OC₁₋₈alkyl,
(5) halogen;
(6) —NR⁵,
(7) —SR⁵, and
(8) —CF₃,
wherein two C₁₋₈alkyl substituents along with the atoms to which they are attached can form a 4- to 8-membered ring;
each R¹⁰ is independently selected from the group consisting of:
(1) —C₁₋₈ alkyl,
(2) —C₂₋₈ alkenyl,
(3) —(CH₂)ₙ-phenyl,
(4) —(CH₂)ₙ-naphthyl,
(5) —(CH₂)ₙ-heteroaryl,
(6) —(CH₂)ₙC₂₋₉heterocycloalkyl,
(7) —(CH₂)ₙC₃₋₇ cycloalkyl,
(8) halogen,
(9) —OR⁸,
wherein alkenyl, phenyl, naphthyl, and heteroaryl are unsubstituted or substituted with one to three substituents independently selected from halogen, hydroxy, C₁₋₄ alkyl, trifluoromethyl, and C₁₋₄ alkoxy, and wherein alkyl, cycloalkyl, heterocycloalkyl, and any methylene (CH₂) carbon atom in R¹⁰ are unsubstituted or substituted with one to two substituents independently selected from halogen, hydroxy, oxo, C₁₋₄ alkyl, trifluoromethyl, and C₁₋₄ alkoxy, or two R¹⁰ substituents on the same carbon atom are taken together with the carbon atom to form a cyclopropyl group;
r is 1 or 2;
s is 0, 1 or 2;
n is 0, 1, 2, 3 or 4, and
p is 0, 1 or 2.

2. A compound of claim 1 wherein R⁹ is hydrogen, and pharmaceutically acceptable salts thereof.

3. A compound of claim 1 wherein R¹ is selected from the group consisting of: amidino, —C₁₋₄alkyliminoyl, —C₁₋₈ alkyl, —(CH₂)ₙN(R⁸)₂, —(CH₂)ₙC₂₋₉heterocycloalkyl, —(CH₂)ₙC₃₋₈cycloalkyl, —(CH₂)ₙphenyl, —(CH₂)ₙnaphthyl, and —(CH₂)ₙheteroaryl, wherein phenyl, naphthyl and heteroaryl are unsubstituted or substituted with one to three substituents independently selected from R³, and alkyl, cycloalkyl and heterocycloalkyl and —(CH₂)ₙ are unsubstituted or substituted with one to three substituents selected from R³ and oxo, and pharmaceutically acceptable salts thereof.

4. A compound of claim 1 wherein R² is phenyl optionally substituted with one to three groups independently selected from R¹⁰, and pharmaceutically acceptable salts thereof.

5. A compound of claim 1 wherein R⁶ is hydrogen, and pharmaceutically acceptable salts thereof.

6. A compound of claim 1 wherein R⁵ is selected from the group consisting of: —C₁₋₆ alkyl, and —(CH₂)₀₋₁C₃₋₈cycloalkyl, wherein alkyl, and cycloalkyl are unsubstituted or substituted with one to three substituents independently selected from R³ and oxo, and wherein any methylene (CH₂) in R⁵ is unsubstituted or substituted with one to two substituents independently selected from halogen, hydroxy, oxo, and C₁₋₄ alkyl, and pharmaceutically acceptable salts thereof.

7. A compounds of claim 1 wherein R⁷ is selected from the group consisting of: —(CH₂)₀₋₂NR⁸C(O)R⁸, —(CH₂)₀₋₂OR⁸, —(CH₂)₀₋₂C≡N, —(CH₂)₀₋₂C(O)OR⁸, —(CH₂)ₙC(O)N(R⁸)₂, —(CH₂)₀₋₂NR⁸C(O)N(R⁸)₂, —(CH₂)₀₋₂NR⁸C(O)heteroaryl, —(CH₂)₀₋₂heteroaryl, —(CH₂)ₙNR⁸S(O)₂R⁸, wherein heteroaryl is unsubstituted or substituted with one to three substituents selected from C₁₋₄ alkyl, and any methylene (CH₂)ₙ in R⁷ is unsubstituted or substituted with one to two substituents independently selected from halogen, hydroxy, oxo, and C₁₋₄ alkyl, or two C₁₋₄ alkyl substituents on any methylene (CH₂)ₙ in R⁷ together with the atom to which they are attached form a 3, 4, 5, or 6-membered ring optionally containing an additional heteroatom selected from O, S, —NH, and —NC₁₋₄ alkyl.

8. A compound of claim 1 wherein R¹⁰ is selected from the group consisting of: —C₁₋₈ alkyl, halogen, —OR⁸, —(CH₂)ₙC≡N, —(CH₂)ₙS(O)ₚR⁸, and —CF₃, wherein any alkyl and methylene (CH₂) carbon atom in R¹⁰ is unsubstituted or substituted with one to two substituents independently selected from halogen, hydroxy, oxo, C₁₋₄ alkyl, trifluoromethyl, and C₁₋₄ alkoxy.

9. A compound of claim 1 wherein Z is CH.

10. A compound of claim 1 wherein Z is N.

11. A compound of claim 1 of structural formula IIa or IIb of the indicated trans relative stereochemical configuration: 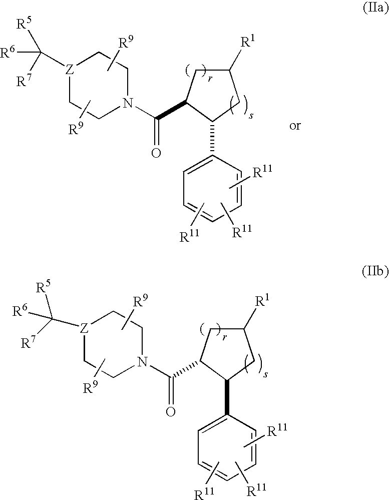
or a pharmaceutically acceptable salt thereof, wherein
Z is N or CR⁴;
R¹ is selected from the group consisting of:
(1) amidino,
(2) —C₁₋₄alkyliminoyl,
(3) —C₁₋₈ alkyl,
(4) —(CH₂)ₙN(R⁸)₂,
(5) —(CH₂)ₙC₂₋₉heterocycloalkyl,
(6) —(CH₂)ₙC₃₋₈cycloalkyl,
(7) —(CH₂)ₙphenyl,
(8) —(CH₂)ₙnaphthyl,
(9) —(CH₂)ₙheteroaryl,
(10) —(CH₂)ₙC(O)C₁₋₈ alkyl,
(11) —(CH₂)ₙC(O)C₃₋₈cycloalkyl,
(12) —(CH₂)ₙC(O)C₂₋₉heterocycloalkyl,
(13) —(CH₂)ₙC(O)phenyl,
(14) —(CH₂)ₙC(O)naphthyl,
(15) —(CH₂)ₙC(O)heteroaryl,
(16) —(CH₂)ₙCO₂H,
(17) —(CH₂)ₙCO₂C₁₋₈ alkyl,
(18) —(CH₂)ₙCO₂C₃₋₈cycloalkyl,
(19) —(CH₂)ₙCO₂C₂₋₉heterocycloalkyl,
(20) —(CH₂)ₙCO₂-phenyl,
(21) —(CH₂)ₙCO₂naphthyl,
(22) —(CH₂)ₙCO₂heteroaryl,
wherein phenyl, naphthyl, and heteroaryl are unsubstituted or substituted with one to three substituents independently selected from R³, and alkyl, cycloalkyl, heterocycloalkyl and (CH₂)ₙ are unsubstituted or substituted with one to three substituents independently selected from R³ and oxo;
R² is selected from the group consisting of:
(1) phenyl,
(2) naphthyl, and
(3) heteroaryl,
wherein phenyl, naphthyl, and heteroaryl are unsubstituted or substituted with one to three substituents independently selected from R¹⁰;
each R³ is independently selected from the group consisting of:
(1) —C₁₋₈ alkyl,
(2) —(CH₂)ₙ-phenyl,
(3) —(CH₂)ₙ-heteroaryl,
(4) —(CH₂)ₙC₂₋₉heterocycloalkyl,
(5) —(CH₂)ₙC₃₋₇ cycloalkyl,
(6) halogen,
(7) —OR⁸,
(8) —(CH₂)ₙC≡N,
(9) —(CH₂)ₙN(R⁸)₂,
(10) —(CH₂)ₙC(O)N(R⁸)₂,
(11) —(CH₂)ₙC(O)NR⁸N(R⁸)₂,
(12) —(CH₂)ₙC(O)NR⁸NR⁸C(O)R⁸, and
(13) —(CH₂)ₙCF₃,
wherein phenyl and heteroaryl are unsubstituted or substituted with one to three substituents independently selected from halogen, hydroxy, C₁₋₄ alkyl, trifluoromethyl, and C₁₋₄ alkoxy, and wherein any alkyl, cycloalkyl, heterocycloalkyl, and methylene (CH₂) carbon atom in R³ is unsubstituted or substituted with one to two substituents independently selected from halogen, hydroxy, oxo, C₁₋₄ alkyl, trifluoromethyl, and C₁₋₄ alkoxy, or two R³ substituents on the same carbon atom are taken together with the carbon atom to form a cyclopropyl group;
R⁴ is selected from the group consisting of:
(1) hydrogen,
(2) —C₁₋₆ alkyl, and
(3) —OC₁₋₆ alkyl;
R⁵ is selected from the group consisting of:
(1) —CF₃,
(2) —C₁₋₆ alkyl,
(3) —C₂₋₈ alkenyl,
(4) —C₂₋₈ alkynyl,
(5) —OC₁₋₈ alkyl,
(6) —(CH₂)ₙC₃₋₈cycloalkyl,
(7) —(CH₂)ₙC₂₋₉heterocycloalkyl,
(8) —(CH₂)ₙ-phenyl,
(9) —(CF₁₂)ₙ-naphthyl, and
(10) —(CH₂)ₙheteroaryl,
wherein phenyl, naphthyl, and heteroaryl are unsubstituted or substituted with one to three substituents independently selected from R³, and alkyl, alkenyl, alkynyl, cycloalkyl and heterocycloalkyl are unsubstituted or substituted with one to three substituents independently selected from R³ and oxo, and wherein any methylene (CH₂) in R⁵ is unsubstituted or substituted with one to two substituents independently selected from halogen, hydroxy, oxo, and C₁₋₄ alkyl;
R⁶ is selected from the group consisting of:
(1) hydrogen,
(2) —C₁₋₆ alkyl, and
(3) —OC₁₋₆ alkyl;
R⁷ is selected from the group consisting of:
(1) —(CH₂)ₙN(R⁸)₂,
(2) —(CH₂)ₙNR⁸C(O)R⁸,
(3) —(CH₂)ₙOR⁸,
(4) —(CH₂)ₙC≡N,
(5) —(CH₂)ₙC(O)OR⁸,
(6) —(CH₂)ₙC(O)N(R⁸)₂,
(7) —(CH₂)ₙNR⁸C(O)N(R⁸)₂,
(8) —(CH₂)ₙNR⁸C(O)heteroaryl,
(9) —(CH₂)ₙheteroaryl,
(10) —(CH₂)ₙNR⁸S(O)ₚR⁸,
(11) —(CH₂)ₙSR⁸, and
(12) —(CH₂)ₙS(O)ₚR⁸,
wherein heteroaryl is unsubstituted or substituted with one to three substituents selected from C₁₋₄ alkyl, and any methylene (CH₂)ₙ in R⁷ is unsubstituted or substituted with one to two substituents independently selected from halogen, hydroxy, oxo, and C₁₋₄ alkyl, or two C₁₋₄alkyl substituents on any methylene (CH₂)ₙ in R⁷ together with the atom to which they are attached form a 3, 4, 5, or 6-membered ring optionally containing an additional heteroatom selected from O, S, —NH, and —NC₁₋₄ alkyl;
each R⁸ is independently selected from the group consisting of:
(1) hydrogen,
(2) —C₁₋₈ alkyl,
(3) —C₂₋₈ alkenyl,
(4) —C₂₋₈ alkynyl,
(5) —OC₁₋₈ alkyl,
(6) —(CH₂)ₙC₃₋₈cycloalkyl,
(7) —(CH₂)ₙC₂₋₉heterocycloalkyl,
(8) —(CH₂)ₙ-phenyl,
(9) —(CH₂)ₙ-naphthyl, and
(10) —(CH₂)ₙheteroaryl,
wherein phenyl, naphthyl, and heteroaryl, alkyl, alkenyl, alkynyl, cycloalkyl and heterocycloalkyl are unsubstituted or substituted with one to three substituents independently selected from N(C₁₋₆alkyl)₂, —NH₂, NH(C₁₋₆ alkyl), halogen, C₁₋₆alkyl, C₁₋₆alkoxy, hydroxy, and oxo, and wherein any methylene (CH₂) in R⁸ is unsubstituted or substituted with one to two substituents independently selected from halogen, hydroxy, oxo, and C₁₋₄ alkyl;
each R⁹ is independently selected from the group consisting of:
(1) hydrogen,
(2) —OH,
(3) C₁₋₈alkyl,
(4) —OC₁₋₈alkyl,
(5) halogen;
(6) —NR⁵,
(7) —SR⁵, and
(8) —CF₃,
wherein two C₁₋₈alkyl substituents along with the atoms to which they are attached can form a 4- to 8-membered ring;
each R11 is independently selected from the group consisting of:
(1) hydrogen,
(2) —C₁₋₈ alkyl,
(3) —C₂₋₈ alkenyl,
(4) —(CH₂)ₙ-phenyl,
(5) —(CH₂)ₙ-naphthyl,
(6) —(CH₂)ₙ-heteroaryl,
(7) —(CH₂)ₙC₂₋₉heterocycloalkyl,
(8) —(CH₂)ₙC₃₋₇ cycloalkyl,
(9) halogen, and
(10) —OR⁸,
wherein alkenyl, phenyl, naphthyl, and heteroaryl are unsubstituted or substituted with one to three substituents independently selected from halogen, hydroxy, C₁₋₄ alkyl, trifluoromethyl, and C₁₋₄ alkoxy, and wherein alkyl, cycloalkyl, heterocycloalkyl, and any methylene (CH₂) carbon atom in R¹¹ are unsubstituted or substituted with one to two substituents independently selected from halogen, hydroxy, oxo, C₁₋₄ alkyl, trifluoromethyl, and C₁₋₄ alkoxy, or two R¹¹ substituents on the same carbon atom are taken together with the carbon atom to form a cyclopropyl group;
r is 1 or 2;
s is 0, 1 or 2;
n is 0, 1, 2, 3 or 4, and
p is 0, 1 or 2.

12. A compound of claim 11 selected from the group consisting of: 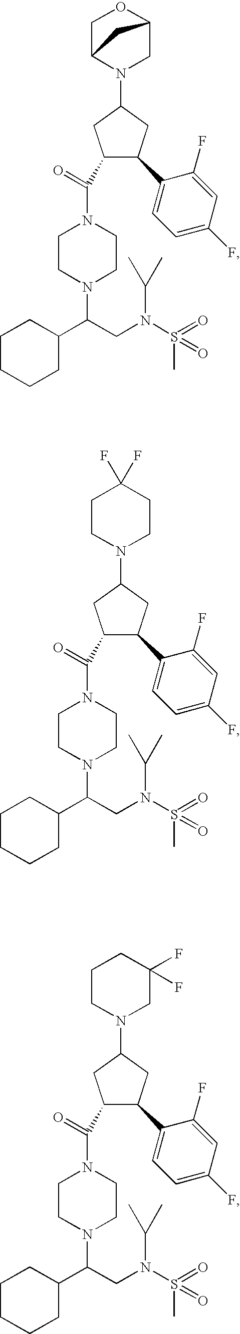 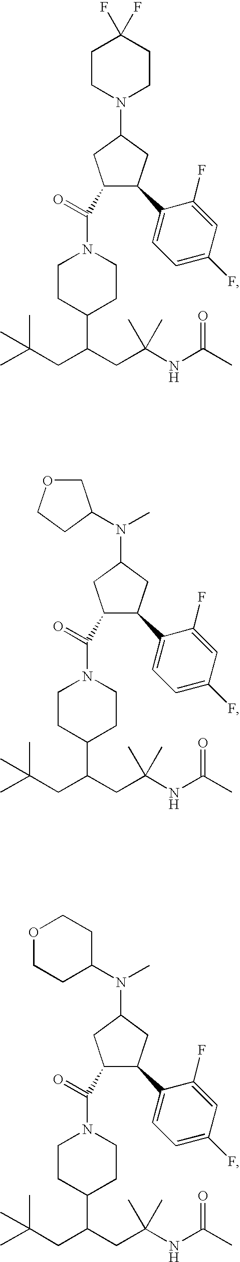 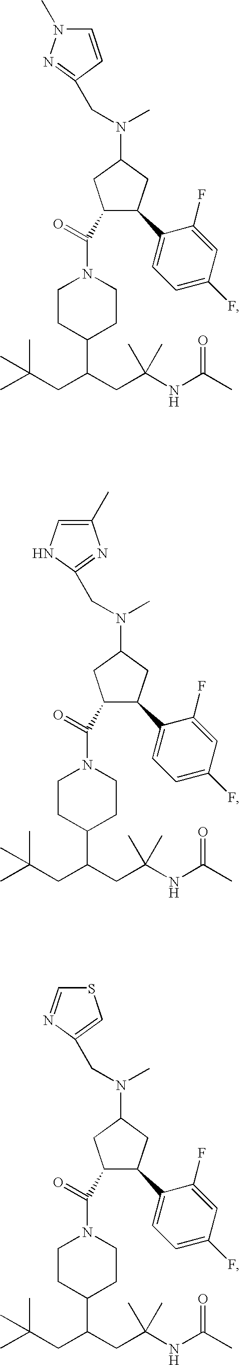 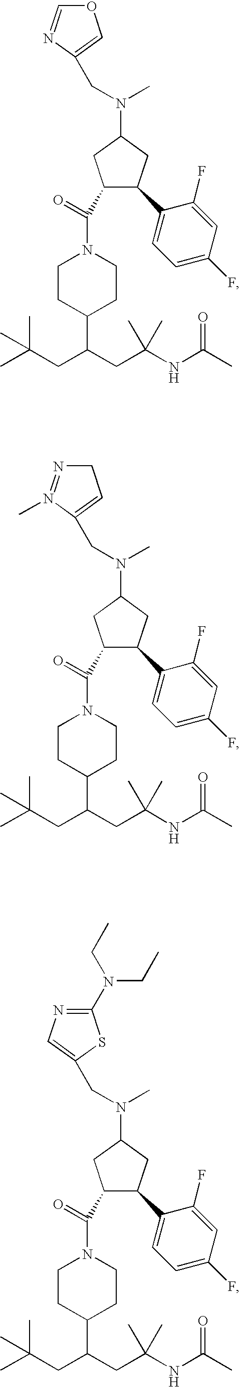 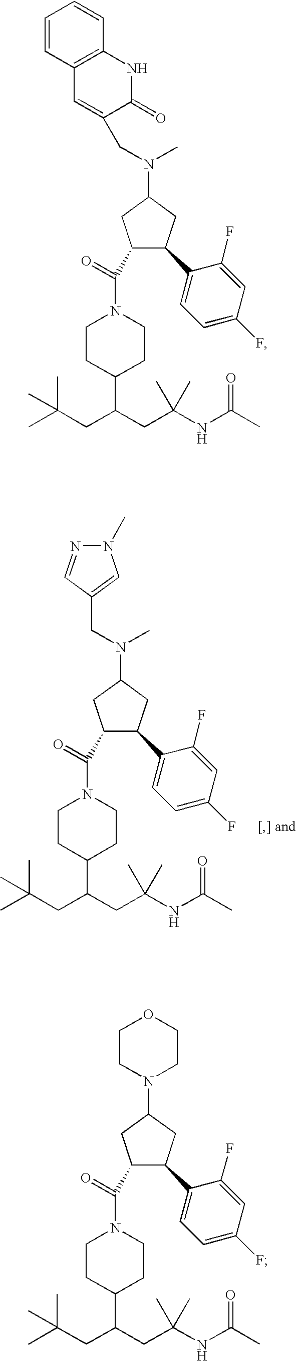
or a pharmaceutically acceptable salt thereof.

13. A compound of claim 12 which is: 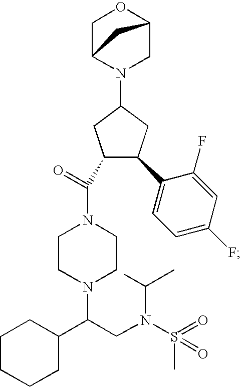
or a pharmaceutically acceptable salt thereof.

14. A compound of claim 12 which is: 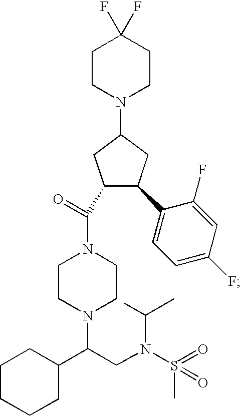
or a pharmaceutically acceptable salt thereof.

15. A compound of claim 12 which is: 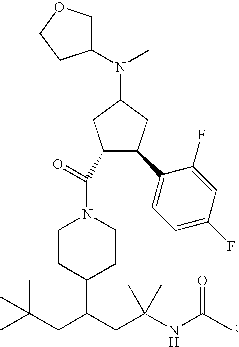
or a pharmaceutically acceptable salt thereof.

16. A compound of claim 12 which is: 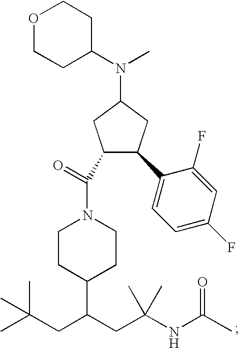
or a pharmaceutically acceptable salt thereof.

17. A pharmaceutical composition which comprises a compound of claim 1 and a pharmaceutically acceptable carrier.

18. A compound of claim 12 wherein the pharmaceutically acceptable salt thereof is the HCl salt.

19-23. (canceled)

24. A method of treating obesity in a human patient in need thereof comprising administering to the patient a compound in accordance with claim 1 in an amount that is effective to treat obesity.
